(19) United States
(12) Patent Application Publication
(10) Pub. No.: US 2026/0069410 A1
(43) Pub. Date: Mar. 12, 2026

(54) IMPLANTABLE PROSTHETIC POSTERIOR MITRAL VALVE

(71) Applicant: ReNiva, Inc., Mountain View, CA (US)

(72) Inventors: Kenneth M. MARTIN, Los Altos Hills, CA (US); Takashi Harry INO, San Jose, CA (US); Christopher Blake FINNEGAN, San Luis Obispo, CA (US); Mark S. LEUNG, Duncan (CA)

(73) Assignee: ReNiva, Inc., Mountain View, CA (US)

(21) Appl. No.: 19/340,368

(22) Filed: Sep. 25, 2025

Related U.S. Application Data
(63) Continuation of application No. PCT/US2024/028936, filed on May 10, 2024.
(60) Provisional application No. 63/501,784, filed on May 12, 2023.

(51) Int. Cl. A61F 2/24

(52) U.S. Cl. CPC A61F 2/2418; A61F 2/2436; A61F 2220/0008; A61F 2230/0006; A61F 2250/0007; A61F 2250/001; A61F 2250/0012

(57) ABSTRACT

An implantable prosthetic posterior mitral valve is described where one variation generally includes a frame. A posterior leaflet having one or more scallops may be connected to the frame such that the scallops extend from the frame for coaptation against a native anterior leaflet when the frame is deployed. At least one arm member may be connected to the frame such that the arm member extends laterally in a curved configuration configured to approximate a curvature of a native mitral annulus when the frame is deployed, and an anchoring leg may be connected to the frame and extend in a superior direction from a posterior side of the frame and defines a capture region between the anchoring leg and the posterior side. The capture region may be sized to receive at least a portion of a native posterior leaflet in an elongated state when the frame is deployed.

### CROSS-REFERENCE TO RELATED APPLICATIONS

This application is a continuation of International Patent Application No. PCT/US2024/028936 filed May 10, 2024, which claims the benefit of priority to U.S. Provisional Application No. 63/501,784 filed May 12, 2023, which is incorporated herein by reference in its entirety.

### BACKGROUND OF THE INVENTION

The mitral valve is located between the left atrium 57 (upper heart chamber that receives oxygenated blood from the lungs) and the left ventricle 58 (the heart's main pumping chamber that pushes blood into the aorta). Normally, blood flows one way, as shown by arrow 71, through the mitral valve's anterior leaflet 63 and posterior leaflet 64, which open to allow blood to enter the left ventricle, and close to prevent blood from leaking backwards from the left ventricle to the left atrium. In mitral valve insufficiency or mitral valve regurgitation, the leaflets 63, 64 do not close tightly and blood leaks back into the left atrium 57, for example due to heart enlargement.

FIG. 1 shows a superior cross-sectional view of a mitral valve 54 of a human heart 51 including components of the mitral valve apparatus and the adjacent structures. FIG. 2 shows a side view of a cross-section of the heart 51. The mitral valve apparatus includes posterior 64 and anterior 63 leaflets, a mitral annulus 62, which forms a ring around the leaflets, and chordae tendineae 65, which tether the valve leaflets to papillary muscles 66, 67 in the left ventricle and prevent them from prolapsing into the left atrium. Dysfunction of any of these portions of the mitral valve apparatus can cause regurgitation. There are several causes that may lead to the degeneration or functional insufficiency of the mitral valve apparatus. The disease is prevalent in approximately 2% of the population and is one of the two most common valvular heart diseases in the elderly and the most common in low- and middle-income countries.

The posterior leaflet 64 of the mitral valve composes approximately ⅗ of the circumference of the mitral annulus 62 and comprises three individual scallops identified as P1 (medial scallop), P2 (middle scallop), and P3 (lateral scallop). The three corresponding segments of the anterior leaflet 63 are A1 (medial segment), A2 (middle segment), and A3 (lateral segment).

Several attempts have been made to treat mitral valve regurgitation including prosthetic mitral valves and mitral clips, which have had various degrees of success. Transvascular approaches improve morbidity and have faster recovery times than surgical approaches but pose some challenges such as difficulty implanting with an endovascular approach often requiring catheters as large as 30 French, which may cause vascular complications or problems traversing and closing the atrial septum, properly securing the device and avoiding migration, implanting a valve that closes fully and does not leak around the valve to fully prevent mitral regurgitation, optimizing Geometric Orifice Area (GOP) as close as possible to Effective Orifice Area (EOA), which reduces the ability of blood to flow properly from the lungs into the heart, creating areas of poor blood flushing, which can create thrombosis. Also, a significant challenge is to avoid applying unnatural forces or disruption to various parts of the heart that may lead to further complications, for example applying compression to the aortic valve, which causes left ventricular outflow tract obstruction (LVOT) impeding egress of blood from the left ventricle to the systemic circulation; stretching the mitral annulus, further exasperating mitral insufficiency; disrupting the chordae tendineae, which may affect the papillary muscles or muscles of the left ventricle causing further deterioration; or compressing arterial or conductive tissues leading to A-V block. A further deficiency of current treatments is that in the case of failed treatment further intervention is not permitted or complicated by presence of a prosthesis attached to the native leaflets.

Thus, there is a need for an improved treatment of mitral valve regurgitation or other valvular disease.

### SUMMARY OF THE INVENTION

This disclosure is related to prosthetic heart valves, in particular prosthetic hemi-mitral valves and delivery systems. The embodiments disclosed herein each have several aspects no single one of which is solely responsible for the disclosure's desirable attributes. Without limiting the scope of this disclosure, its more prominent features will now be briefly discussed. After considering this discussion, and particularly after reading the section entitled “Detailed Description,” one will understand how the features of the embodiments described herein provide advantages over existing systems, devices and methods for mechanical circulatory support systems.

Statements of the disclosure include:

Statement 1. A mitral valve repair apparatus, comprising a frame having an annulus section, an atrial section extending from a superior aspect of the frame, and a ventricle section extending from an inferior aspect of the frame, wherein the annulus section defines an arc length which is sized to be less than a major arc between native commissures of a native mitral valve annulus; a posterior leaflet having one or more scallops connected to the frame such that the one or more scallops extend from the frame for coaptation against a native anterior leaflet when the frame is deployed; at least one arm member connected to the frame such that the at least one arm member extends laterally in a curved configuration configured to approximate a curvature of a native mitral annulus when the frame is deployed; and an anchoring leg connected to the frame such that the anchoring leg extends in a superior direction from a posterior side of the frame and defines a capture region between the anchoring leg and the posterior side where the capture region is sized to receive at least a portion of a native posterior leaflet in an elongated state when the frame is deployed.

Statement 2. The apparatus of Statement 1, wherein the at least one arm member comprises a first arm and a second arm each extending laterally from the frame.

Statement 3. The apparatus of any of the preceding Statements 1 to 2, wherein the first arm member comprises a first anchor point and the second arm member comprises a second anchor point where each anchor point is configured to align respectively with or in proximity to a medial and a lateral trigone of a left atrium of a subject.

Statement 4. The apparatus of any of the preceding Statements 1 to 3, further comprising a first anchor for securing the first anchor point to the left atrium.

Statement 5. The apparatus of any of the preceding Statements 1 to 4, wherein the first anchor comprises a corkscrew anchor and tip pillar having at least one deployable barb.

Statement 6. The apparatus of any of the preceding Statements 1 to 5 wherein the corkscrew anchor further comprises a tip pillar.

Statement 7. The apparatus of any of the preceding Statements 1 to 3, wherein each of the first and second anchor points comprise the medial and the lateral trigone and an area having a radius of 3 mm surrounding each of the medial and lateral trigones.

Statement 8. The apparatus of any of the preceding Statements 1 to 3, wherein the first and second anchor points each comprise an opening for accommodating a position of the medial and the lateral trigones.

Statement 9. The apparatus of any of the preceding Statements 1 to 8, wherein the first and second anchor points each comprise a slot for accommodating the position of the medial and the lateral trigones.

Statement 10. The apparatus of Statements 1, wherein at least a portion of the mitral valve repair apparatus comprises a membrane or fabric layer.

Statement 11. The apparatus of Statement 1, wherein the one or more scallops comprise a P1, P2, and P3 scallop each connected to the frame.

Statement 12. The apparatus of Statement 1, wherein the posterior leaflet has a width configured to span an arc length of between 0 to 100% of the native posterior leaflet.

Statement 13. The apparatus of any of the preceding Statements 1 to 12, wherein the width of the posterior leaflet is configured to span the arc length of between 30 to 80% of the native posterior leaflet.

Statement 14. The apparatus of any of the preceding Statements 1 to 12,wherein the width of the posterior leaflet is configured to span the arc length of between 50 to 75% of the native posterior leaflet.

Statement 15. The apparatus of Statement 1, wherein the posterior leaflet is sized to cover a native P2 scallop and a portion of a native P1 scallop and a portion of a native P3 scallop.

Statement 16. The apparatus of any of the preceding Statements 1 to 15, wherein the posterior leaflet is sized to cover 30% to 70% of the native P1 scallop.

Statement 17. The apparatus of any of the preceding Statements 1 to 16, wherein the posterior leaflet is sized to cover 30% to 40%, 40% to 60%, or 50% to 70% of the native P1 scallop.

Statement 18. The apparatus of any of the preceding Statements 1 to 15, wherein the posterior leaflet is sized to cover 30% to 70% of the native P3 scallop.

Statement 19. The apparatus of any of the preceding Statements 1 to 16, wherein the posterior leaflet is sized to cover 30% to 40%, 40% to 60%, or 50% to 70%, of the native P3 scallop.

Statement 20. The apparatus of Statement 1, wherein the arc length is sized to be 30% to 90% of a circumference of the native mitral valve annulus.

Statement 21. The apparatus of any of the preceding Statements 1 to 20, wherein the arc length is sized to be 30% to 80%, 40% to 80%, or 50% to 70% of the circumference of the native mitral valve annulus.

Statement 22. The apparatus of Statement 1, wherein the posterior leaflet is configured to define pleats which separate the one or more scallops from one another.

Statement 23. The apparatus of any of the preceding Statements 1 to 22, wherein the pleats are each comprised of a sewn border separating the one or more scallops.

Statement 24. The apparatus of any of the preceding Statements 1 to 22, wherein the posterior leaflet is formed from a singular material shaped into the one or more scallops.

Statement 25. The apparatus of any of the preceding Statements 1 to 22, wherein an interior of each of the one or more scallops form a continuous space with an adjacent scallop of the one or more scallops.

Statement 26. The apparatus of Statement 1, wherein the at least one arm member is configured to be adjustable.

Statement 27. The apparatus of any of the preceding Statements 1 to 26, wherein the at least one arm member is adjustable in arc length, radius of curvature, tension, compression, tension, compression, tension, compression, or flexion.

Statement 28. The apparatus of any of the preceding Statements 1 to 26, further comprising a locking mechanism for securing a configuration of the at least one arm member.

Statement 29. The apparatus of Statement 1, wherein the anchoring leg has a width sized for introduction between a chordae tendinea bundle of a lateral papillary muscle and a chordae tendinea bundle of a medial papillary muscle of a subject.

Statement 30. The apparatus of any of the preceding Statements 1 to 29, wherein the width ranges between 2 to 8 mm.

Statement 31. The apparatus of Statement 1, wherein the anchoring leg comprises a first leg attachment connected to a first portion of the frame and a second leg attachment connected to a second portion of the frame such that the anchoring leg has a middle section defining a radially outward bend.

Statement 32. The apparatus of Statement 1, further comprising one or more additional leg members connected to the frame and in proximity to the anchoring leg for securing a position of the frame to the native mitral annulus.

Statement 33. The apparatus of any of the preceding Statements 1 to 32, wherein the one or more additional leg members comprise two additional leg members positioned on each side of the anchoring leg.

Statement 34. The apparatus of any of the preceding Statements 1 to 32, wherein each additional leg member extends from the frame at a second level which is superior to a first level of the anchoring leg.

Statement 35. The apparatus of any of the preceding Statements 1 to 34, where a difference between the first level and the second level is between 1 mm to 10 mm.

Statement 36. The apparatus of Statement 1, further comprising an arm mount delivery system for deploying the mitral valve repair apparatus.

Statement 37. The apparatus of any of the preceding Statements 1 to 36, wherein the arm mount delivery system comprises an arm mount having configurable wing members connectable to a distal end of the at least one arm member.

Statement 38. The apparatus of any of the preceding Statements 1 to 37, further comprising a delivery sheath connected to the arm mount via a body member from which the wing members extend.

Statement 39. The apparatus of any of the preceding Statements 1 to 38, wherein the wing members are configurable from a delivery configuration where the wing members are retracted relative to the delivery sheath and a deployment configuration where the wing members are extended relative to the delivery sheath such that the at least one arm member is positioned into a deployment configuration.

The features from any of the Statements 1 to 39 can be combined in any number of combinations and in any order and such variations are intended to be within the scope of this disclosure.

Statement 40. A mitral valve repair apparatus, comprising a frame having an annulus section, an atrial section extending from a superior aspect of the frame, and a ventricle section extending from an inferior aspect of the frame; a posterior leaflet having one or more scallops connected to the frame such that the one or more scallops extend from the frame for coaptation against a native anterior leaflet when the frame is deployed, wherein the posterior leaflet defines an exterior arc length defined by an edge of the one or more scallops which contact the native anterior leaflet and the posterior leaflet further defines an interior arc length defined by the annulus section of the frame between scallop commissures such that the exterior arc length is less than or equal to the interior arc length; at least one arm member connected to the frame such that the at least one arm member extends laterally in a curved configuration configured to approximate a curvature of a native mitral annulus when the frame is deployed; and an anchoring leg connected to the frame such that the anchoring leg extends in a superior direction from a posterior side of the frame and defines a capture region between the anchoring leg and the posterior side where the capture region is sized to receive at least a portion of a native posterior leaflet in an elongated state when the frame is deployed.

Statement 41. The apparatus of Statement 40, wherein the exterior arc length is no more than 5% greater than the interior arc length.

Statement 42. The apparatus of any of the preceding Statements 40 to 41, a P1/P2 commissure of the one or more scallops is positioned relatively closer to a central axis defined by the mitral valve repair apparatus than a side commissure of the P1 scallop which is connected to the frame.

Statement 43. The apparatus of Statement 40, a P2/P3 commissure of the one or more scallops is positioned relatively closer to a central axis defined by the mitral valve repair apparatus than a side commissure of the P3 scallop which is connected to the frame.

Statement 44. The apparatus of Statement 40, wherein the at least one arm member comprises a first arm and a second arm each extending laterally from the frame.

Statement 45. The apparatus of any of the preceding Statements 40 to 44, wherein the first arm member comprises a first anchor point and the second arm member comprises a second anchor point where each anchor point is configured to align respectively with or in proximity to a medial and a lateral trigone of a left atrium of a subject.

Statement 46. The apparatus of any of the preceding Statements 40 to 45, further comprising a first anchor for securing the first anchor point to the left atrium.

Statement 47. The apparatus of any of the preceding Statements 40 to 46, wherein the first anchor comprises a corkscrew anchor and at least one deployable barb.

Statement 48. The apparatus of any of the preceding Statements 40 to 47 wherein the corkscrew anchor further comprises a tip pillar.

Statement 49. The apparatus of Statement 40, wherein the posterior leaflet is sized to cover a native P2 scallop and a portion of a native P1 scallop and a portion of a native P3 scallop.

Statement 50. The apparatus of any of the preceding Statements 40 to 49, wherein the posterior leaflet is sized to cover 30% to 70% of the native P1 scallop.

Statement 51. The apparatus of any of the preceding Statements 40 to 50, wherein the posterior leaflet is sized to cover 30% to 40%, 40% to 60%, or 50% to 70% of the native P1 scallop.

Statement 52. The apparatus of any of the preceding Statements 40 to 49, wherein the posterior leaflet is sized to cover 30% to 70% of the native P3 scallop.

Statement 53. The apparatus of any of the preceding Statements 40 to 52, wherein the posterior leaflet is sized to cover 30% to 40%, 40% to 60%, or 50% to 70%, of the native P3 scallop.

Statement 54. The apparatus of Statement 40, wherein the arc length is sized to be 30% to 90% of a circumference of the native mitral valve annulus.

Statement 55. The apparatus of any of the preceding Statements 40 to 54, wherein the arc length is sized to be 30% to 80%, 40% to 80%, or 50% to 70% of the circumference of the native mitral valve annulus.

Statement 56. The apparatus of Statement 40, wherein the posterior leaflet is configured to define pleats which separate the one or more scallops from one another.

Statement 57. The apparatus of any of the preceding Statements 40 to 56, wherein the pleats are each comprised of a sewn border separating the one or more scallops.

Statement 58. The apparatus of any of the preceding Statements 40 to 56, wherein the posterior leaflet is formed from a singular material shaped into the one or more scallops.

Statement 59. The apparatus of any of the preceding Statements 40 to 56, wherein an interior of each of the one or more scallops form a continuous space with an adjacent scallop of the one or more scallops.

Statement 60. The apparatus of Statement 40, wherein the at least one arm member is configured to be adjustable.

Statement 61. The apparatus of any of the preceding Statements 40 to 60, wherein the at least one arm member is adjustable in arc length, radius of curvature, tension, compression, or flexion.

Statement 62. The apparatus of any of the preceding Statements 40 to 61, further comprising a locking mechanism for securing a configuration of the at least one arm member.

Statement 63. The apparatus of Statement 40, further comprising one or more additional leg members connected to the frame and in proximity to the anchoring leg for securing a position of the frame to the native mitral annulus.

Statement 64. The apparatus of any of the preceding Statements 40 to 63, wherein the one or more additional leg members comprise two additional leg members positioned on each side of the anchoring leg.

Statement 65. The apparatus of Statement 63, wherein each additional leg member extends from the frame at a second level which is superior to a first level of the anchoring leg.

Statement 66. The apparatus of any of the preceding Statements 40 to 65, where a difference between the first level and the second level is between 1 mm to 10 mm.

Statement 67. The apparatus of Statement 40, further comprising an arm mount delivery system for deploying the mitral valve repair apparatus.

Statement 68. The apparatus of any of the preceding Statements 40 to 67, wherein the arm mount delivery system comprises an arm mount having configurable wing members connectable to a distal end of the at least one arm member.

Statement 69. The apparatus of any of the preceding Statements 40 to 68, further comprising a delivery sheath connected to the arm mount via a body member from which the wing members extend.

Statement 70. The apparatus of any of the preceding Statements 40 to 69, wherein the wing members are configurable from a delivery configuration where the wing members are retracted relative to the delivery sheath and a deployment configuration where the wing members are extended relative to the delivery sheath such that the at least one arm member is positioned into a deployment configuration.

The features from any of the Statements 40 to 70 can be combined in any number of combinations and in any order and such variations are intended to be within the scope of this disclosure.

Statement 71. A mitral valve repair apparatus, comprising a frame having an annulus section, an atrial section extending from a superior aspect of the frame, and a ventricle section extending from an inferior aspect of the frame; a posterior leaflet having one or more scallops connected to the frame such that the one or more scallops extend from the frame for coaptation against a native anterior leaflet when the frame is deployed; a first arm member connected to a first portion of the frame and a second arm member connected to a second portion of the frame such that the first arm member and the second arm member each extend laterally in a curved configuration opposite to one another to each approximate a curvature of a native mitral annulus when the frame is deployed; a first anchor point located along the first arm member and a second anchor point located along the second arm member, wherein the first anchor point and the second anchor point are each positioned to coincide with a respective first tissue location and a second tissue location; and an anchoring leg connected to the frame such that the anchoring leg extends in a superior direction from a posterior side of the frame and an apex of the anchoring leg defines a bend angle which curves away from the frame when deployed, wherein the anchoring leg and the posterior side define a capture region therebetween which is sized to receive at least a portion of a native posterior leaflet in an elongated state when the frame is deployed.

Statement 72. The apparatus of Statement 71, wherein the first arm member comprises a first anchor point and the second arm member comprises a second anchor point where each anchor point is configured to align respectively with or in proximity to a medial and a lateral trigone of a left atrium of a subject.

Statement 73. The apparatus of any of the preceding Statements 71 to 72, wherein each of the first and second anchor points comprise the medial and the lateral trigone and an area having a radius of 3 mm surrounding each of the medial and lateral trigones.

Statement 74. The apparatus of any of the preceding Statements 71 to 72, wherein the first and second anchor points each comprise an opening for accommodating a position of the medial and the lateral trigones.

Statement 75. The apparatus of any of the preceding Statements 71 to 74, wherein the first and second anchor points each comprise a slot for accommodating the position of the medial and the lateral trigones.

Statement 76. The apparatus of Statements 71, wherein at least a portion of the mitral valve repair apparatus comprises a membrane or fabric layer.

Statement 77. The apparatus of Statement 71, wherein the one or more scallops comprise a P1, P2, and P3 scallop each connected to the frame.

Statement 78. The apparatus of Statement 71, wherein the posterior leaflet has a width configured to span an arc length of between 0 to 100% of the native posterior leaflet.

Statement 79. The apparatus of any of the preceding Statements 71 to 78, wherein the width of the posterior leaflet is configured to span the arc length of between 30 to 80% of the native posterior leaflet.

Statement 80. The apparatus of any of the preceding Statements 71 to 78, wherein the width of the posterior leaflet is configured to span the arc length of between 50 to 75% of the native posterior leaflet.

Statement 81. The apparatus of Statement 71, wherein the anchoring leg has a width sized for introduction between a chordae tendinea bundle of a lateral papillary muscle and a chordae tendinea bundle of a medial papillary muscle of a subject.

Statement 82. The apparatus of any of the preceding Statements 71 to 81, wherein the width ranges between 2 to 8 mm.

Statement 83. The apparatus of Statement 71, wherein the anchoring leg comprises a first leg attachment connected to a first portion of the frame and a second leg attachment connected to a second portion of the frame such that the anchoring leg has a middle section defining a radially outward bend.

The features from any of the Statements 71 to 83 can be combined in any number of combinations and in any order and such variations are intended to be within the scope of this disclosure.

Statement 84. A method for repairing a mitral valve, comprising positioning a frame in a delivery configuration within a delivery sheath into proximity of a mitral valve, the frame having an annulus section, an atrial section extending from a superior aspect of the frame, and a ventricle section extending from an inferior aspect of the frame, wherein the annulus section defines an arc length which is sized to be less than a major arc between native commissures of a native mitral valve annulus; advancing an anchoring leg connected to the frame distally from the delivery sheath until the anchoring leg retracts from a first delivery configuration to a second retracted configuration such that the anchoring leg extends in a superior direction from a posterior side of the frame; introducing the anchoring leg between a chordae tendinea bundle of a lateral papillary muscle and a chordae tendinea bundle of a medial papillary muscle; deploying the frame into an expanded deployment configuration against a native mitral annulus such that a posterior leaflet having one or more scallops connected to the frame extends from the frame for coaptation against a native anterior leaflet; and positioning a first arm member connected to a first portion of the frame and a second arm member connected to a second portion of the frame about a native mitral annulus such that the first arm member and the second arm member each extend laterally in a curved configuration opposite to one another.

Statement 85. The method of Statement 84, wherein positioning the frame comprises intravascularly advancing the frame into a position superior to the mitral valve in a subject.

Statement 86. The method of Statement 84, wherein deploying the frame further comprises securing a native posterior leaflet within a capture region between the anchoring leg and the posterior side of the frame.

Statement 87. The method of any of the preceding Statements 84 to 86, wherein deploying the frame comprises deploying the posterior leaflet to have a width which spans an arc length of between 0 to 100% of the native posterior leaflet.

Statement 88. The method of any of the preceding Statements 84 to 87, wherein deploying the frame comprises deploying the posterior leaflet to have a width which spans an arc length of between 30 to 80% of the native posterior leaflet.

Statement 89. The method of any of the preceding Statements 84 to 86, wherein deploying the frame comprises deploying the posterior leaflet to have a width which spans an arc length of between 50 to 75% of the native posterior leaflet.

Statement 90. The method of Statement 84, wherein advancing the anchoring leg comprises retracting the anchoring leg from the first delivery configuration to the second retracted configuration within a ventricle of the subject.

Statement 91. The method of Statement 84, wherein deploying the frame comprises deploying the frame such that the ventricle section is deployed inferior to the native mitral annulus, the annulus section is deployed against a portion of the native mitral annulus, and the atrial section is deployed superior to the native mitral annulus.

Statement 92. The method of Statement 84, wherein positioning the first arm member further comprises securing a first anchor point located along the first arm member to or in proximity to a first trigone tissue region and a second anchor point located along the second arm member to or in proximity to a second trigone tissue region.

Statement 93. The method of any of the preceding Statements 84 to 92, wherein each of the first and second anchor points comprise the medial and the lateral trigone and an area having a radius of 3 mm surrounding each of the medial and lateral trigones.

Statement 94. The method of any of the preceding Statements 84 to 92, wherein securing the first anchor point comprises attaching the first anchor point to the first trigone tissue region via a first tissue anchor and attaching the second anchor point to the second trigone tissue region via a second tissue anchor.

Statement 95. The method of any of the preceding Statements 84 to 94, wherein positioning the first arm member comprises securing a first anchor to the native mitral annulus for securing the first arm member.

Statement 96. The method of any of the preceding Statements 84 to 95, wherein securing the first anchor comprises securing a corkscrew anchor and at least one deployable barb into the native mitral annulus.

Statement 97. The method of any of the preceding Statements 84 to 96 wherein the corkscrew anchor further comprises a tip pillar.

Statement 98. The method of Statement 84, wherein deploying the frame comprises deploying the posterior leaflet which is sized to cover a native P2 scallop and a portion of a native P1 scallop and a portion of a native P3 scallop.

Statement 99. The method of any of the preceding Statements 84 to 98, wherein the posterior leaflet is sized to cover 30% to 70% of the native P1 scallop.

Statement 100. The method of any of the preceding Statements 84 to 99, wherein the posterior leaflet is sized to cover 30% to 40%, 40% to 60%, or 50% to 70% of the native P1 scallop.

Statement 101. The method of any of the preceding Statements 84 to 98, wherein deploying the frame comprises deploying the posterior leaflet which is sized to cover 30% to 70% of the native P3 scallop.

Statement 102. The method of any of the preceding Statements 84 to 101, wherein deploying the frame comprises deploying the posterior leaflet which is sized to cover 30% to 40%, 40% to 60%, or 50% to 70%, of the native P3 scallop.

Statement 103. The method of Statement 84, wherein the arc length is sized to be 30% to 90% of a circumference of the native mitral valve annulus.

Statement 104. The method of any of the preceding Statements 84 to 103, wherein the arc length is sized to be 30% to 80%, 40% to 80%, or 50% to 70% of the circumference of the native mitral valve annulus.

Statement 105. The method of Statement 84, wherein deploying the frame comprises deploying the posterior leaflet which is configured to define pleats which separate the one or more scallops from one another.

Statement 106. The method of any of the preceding Statements 84 to 105, wherein the pleats are each comprised of a sewn border separating the one or more scallops.

Statement 107. The method of any of the preceding Statements 84 to 105, wherein the posterior leaflet is formed from a singular material shaped into the one or more scallops.

Statement 108. The method of any of the preceding Statements 84 to 105, wherein an interior of each of the one or more scallops form a continuous space with an adjacent scallop of the one or more scallops.

Statement 109. The method of Statement 84, wherein positioning the first arm member comprises positioning the at least one arm member which is configured to be adjustable.

Statement 110. The method of any of the preceding Statements 84 to 109, wherein the at least one arm member is adjustable in arc length, radius of curvature, tension, compression, or flexion.

Statement 111. The method of any of the preceding Statements 84 to 109, further comprising deploying a locking mechanism for securing a configuration of the at least one arm member.

Statement 112. The method of Statement 84, further comprising deploying one or more additional leg members connected to the frame and in proximity to the anchoring leg for securing a position of the frame to the native mitral annulus.

Statement 113. The method of any of the preceding Statements 84 to 112, wherein the one or more additional leg members comprise two additional leg members positioned on each side of the anchoring leg.

Statement 114. The method of any of the preceding Statements 84 to 112, wherein each additional leg member extends from the frame at a second level which is superior to a first level of the anchoring leg.

Statement 115. The method of any of the preceding Statements 84 to 114, where a difference between the first level and the second level is between 1 mm to 10 mm.

Statement 116. The method of any of the preceding Statements 84 to 84, positioning the frame further comprises positioning an arm mount delivery system into proximity of the mitral valve for deploying the mitral valve repair apparatus.

Statement 117. The method of any of the preceding Statements 84 to 116, wherein the arm mount delivery system comprises an arm mount having configurable wing members connectable to a distal end of the at least one arm member.

Statement 118. The method of any of the preceding Statements 84 to 117, further comprising deploying a delivery sheath connected to the arm mount via a body member from which the wing members extend.

Statement 119. The method of any of the preceding Statements 84 to 118, wherein the wing members are configurable from a delivery configuration where the wing members are retracted relative to the delivery sheath and a deployment configuration where the wing members are extended relative to the delivery sheath such that the at least one arm member is positioned into a deployment configuration.

The features from any of the Statements 84 to 119 can be combined in any number of combinations and in any order and such variations are intended to be within the scope of this disclosure.

Statement 120. An apparatus for repairing a mitral valve according to any of the features from any of the Statements 1-119 where any of the features are combined in any number of combinations and in any order.

Statement 121: A mitral valve repair apparatus, comprising a frame having an annulus section, an atrial section extending from a superior aspect of the frame, and a ventricle section extending from an inferior aspect of the frame; a posterior leaflet having one or more scallops connected to the frame such that the one or more scallops extend from the frame for coaptation against a native anterior leaflet when the frame is deployed; at least one arm member connected to the frame such that the at least one arm member extends laterally in a curved configuration configured to approximate a curvature of a native mitral annulus when the frame is deployed; and an anchoring leg connected to the frame such that the anchoring leg extends in a superior direction from a posterior side of the frame and defines a capture region between the anchoring leg and the posterior side where the capture region is sized to receive at least a portion of a native posterior leaflet in an elongated state when the frame is deployed.

Statement 122: The apparatus of Statement 121, wherein the at least one arm member comprises a first arm and a second arm each extending laterally from the frame.

Statement 123: The apparatus of any of Statements 121-122, wherein the first arm member comprises a first anchor point and the second arm member comprises a second anchor point where each anchor point is configured to align respectively with or in proximity to a medial and a lateral trigone of a left atrium of a subject.

Statement 124: The apparatus of Statements 123, wherein each of the first and second anchor points comprise the medial and the lateral trigone and an area having a radius of 3 mm surrounding each of the medial and lateral trigones.

Statement 125: The apparatus of any of Statements 123-124, wherein the first and second anchor points each comprise an opening for accommodating a position of the medial and the lateral trigones.

Statement 126: The apparatus of any of Statements 121-125, wherein the first and second anchor points each comprise a slot for accommodating the position of the medial and the lateral trigones.

Statement 127: The apparatus of any of Statements 121-126, wherein at least a portion of the mitral valve repair apparatus comprises a membrane or fabric layer.

Statement 128: The apparatus of any of Statements 121-127, wherein the one or more scallops comprise a P1, P2, and P3 scallop each connected to the frame.

Statement 129: The apparatus of any of Statements 121-128, wherein the posterior leaflet has a width configured to span an arc length of between 0 to 100% of the native posterior leaflet.

Statement 130: The apparatus of Statement 129, wherein the width of the posterior leaflet is configured to span the arc length of between 30 to 80% of the native posterior leaflet.

Statement 131: The apparatus of Statement 129, wherein the width of the posterior leaflet is configured to span the arc length of between 50 to 75% of the native posterior leaflet.

Statement 132: The apparatus of any of Statements 121-131, wherein the anchoring leg has a width sized for introduction between a chordae tendinea bundle of a lateral papillary muscle and a chordae tendinea bundle of a medial papillary muscle of a subject.

Statement 133: The apparatus of Statement 12, wherein the width ranges between 2 to 8 mm.

Statement 134: The apparatus of any of Statements 121-133, wherein the anchoring leg comprises a first leg attachment connected to a first portion of the frame and a second leg attachment connected to a second portion of the frame such that the anchoring leg has a middle section defining a radially outward bend.

Statement 135: A mitral valve repair apparatus, comprising a frame having an annulus section, an atrial section extending from a superior aspect of the frame, and a ventricle section extending from an inferior aspect of the frame; a posterior leaflet having one or more scallops connected to the frame such that the one or more scallops extend from the frame for coaptation against a native anterior leaflet when the frame is deployed; a first arm member connected to a first portion of the frame and a second arm member connected to a second portion of the frame such that the first arm member and the second arm member each extend laterally in a curved configuration opposite to one another to each approximate a curvature of a native mitral annulus when the frame is deployed; a first anchor point located along the first arm member and a second anchor point located along the second arm member, wherein the first anchor point and the second anchor point are each positioned to coincide with a respective first tissue location and a second tissue location; and an anchoring leg connected to the frame such that the anchoring leg extends in a superior direction from a posterior side of the frame and an apex of the anchoring leg defines a bend angle which curves away from the frame when deployed, wherein the anchoring leg and the posterior side define a capture region therebetween which is sized to receive at least a portion of a native posterior leaflet in an elongated state when the frame is deployed.

Statement 136: The apparatus of Statement 135, wherein the first arm member comprises a first anchor point and the second arm member comprises a second anchor point where each anchor point is configured to align respectively with or in proximity to a medial and a lateral trigone of a left atrium of a subject.

Statement 137: The apparatus of Statement 136, wherein each of the first and second anchor points comprise the medial and the lateral trigone and an area having a radius of 3 mm surrounding each of the medial and lateral trigones.

Statement 138: The apparatus of any of Statements 135-137, wherein the first and second anchor points each comprise an opening for accommodating a position of the medial and the lateral trigones.

Statement 139: The apparatus of Statement 138, wherein the first and second anchor points each comprise a slot for accommodating the position of the medial and the lateral trigones.

Statement 140: The apparatus of any of Statements 135-139, wherein at least a portion of the mitral valve repair apparatus comprises a membrane or fabric layer.

Statement 141: The apparatus of any of Statements 135-140, wherein the one or more scallops comprise a P1, P2, and P3 scallop each connected to the frame.

Statement 142: The apparatus of any of Statements 135-141, wherein the posterior leaflet has a width configured to span an arc length of between 0 to 100% of the native posterior leaflet.

Statement 143: The apparatus of Statement 142, wherein the width of the posterior leaflet is configured to span the arc length of between 30 to 80% of the native posterior leaflet.

Statement 144: The apparatus of Statement 142, wherein the width of the posterior leaflet is configured to span the arc length of between 50 to 75% of the native posterior leaflet.

Statement 145: The apparatus of any of Statements 135-144, wherein the anchoring leg has a width sized for introduction between a chordae tendinea bundle of a lateral papillary muscle and a chordae tendinea bundle of a medial papillary muscle of a subject.

Statement 146: The apparatus of Statement 145, wherein the width ranges between 2 to 8 mm.

Statement 147: The apparatus of any of Statements 135-146, wherein the anchoring leg comprises a first leg attachment connected to a first portion of the frame and a second leg attachment connected to a second portion of the frame such that the anchoring leg has a middle section defining a radially outward bend.

Statement 148: A method for repairing a mitral valve, comprising positioning a frame in a delivery configuration within a delivery sheath into proximity of a mitral valve, the frame having an annulus section, an atrial section extending from a superior aspect of the frame, and a ventricle section extending from an inferior aspect of the frame; advancing an anchoring leg connected to the frame distally from the delivery sheath until the anchoring leg retracts from a first delivery configuration to a second retracted configuration such that the anchoring leg extends in a superior direction from a posterior side of the frame; introducing the anchoring leg between a chordae of a lateral papillary muscle and a chordae of a medial papillary muscle; deploying the frame into an expanded deployment configuration against a native mitral annulus such that a posterior leaflet having one or more scallops connected to the frame extends from the frame for coaptation against a native anterior leaflet; and positioning a first arm member connected to a first portion of the frame and a second arm member connected to a second portion of the frame about a native mitral annulus such that the first arm member and the second arm member each extend laterally in a curved configuration opposite to one another.

Statement 149: The method of Statement 148, wherein positioning the frame comprises intravascularly advancing the frame into a position superior to the mitral valve in a subject.

Statement 150: The method of any of Statements 148-149, wherein deploying the frame further comprises securing a native posterior leaflet within a capture region between the anchoring leg and the posterior side of the frame.

Statement 151: The method of Statement 150, wherein deploying the frame comprises deploying the posterior leaflet to have a width which spans an arc length of between 0 to 100% of the native posterior leaflet.

Statement 152: The method of any of Statements 148-151, wherein deploying the frame comprises deploying the posterior leaflet to have a width which spans an arc length of between 30 to 80% of the native posterior leaflet.

Statement 153: The method of any of Statements 148-151, wherein deploying the frame comprises deploying the posterior leaflet to have a width which spans an arc length of between 50 to 75% of the native posterior leaflet.

Statement 154: The method of any of Statements 148-153, wherein advancing the anchoring leg comprises retracting the anchoring leg from the first delivery configuration to the second retracted configuration within a ventricle of the subject.

Statement 155: The method of any of Statements 148-154, wherein deploying the frame comprises deploying the frame such that the ventricle section is deployed inferior to the native mitral annulus, the atrial section is deployed against a portion of the native mitral annulus, and the atrial section is deployed superior to the native mitral annulus.

Statement 156: The method of any of Statements 148-155, wherein positioning the first arm member further comprises securing a first anchor point located along the first arm member to or in proximity to a first trigone tissue region and a second anchor point located along the second arm member to or in proximity to a second trigone tissue region.

Statement 157: The method of Statement 156, wherein each of the first and second anchor points comprise the medial and the lateral trigone and an area having a radius of 3 mm surrounding each of the medial and lateral trigones.

Statement 158: The method of Statement 156, wherein securing the first anchor point comprises attaching the first anchor point to the first trigone tissue region via a first tissue anchor and attaching the second anchor point to the second trigone tissue region via a second tissue anchor.

Statement 159: A method of treating mitral valve regurgitation, comprising implanting a prosthetic half mitral valve that replaces a portion of the native posterior scallops and preserves a remaining portion of the native posterior leaflets.

Statement 160: The method of Statement 159, wherein the portion of the native posterior leaflets comprises an entire P2 scallop, and a portion of each of a P1 and P3 scallop in a range of 50 to 75%.

Statement 161: The method of any of Statements 159-160, wherein a remaining portion of the native posterior leaflet continues to function when the prosthetic half mitral valve is implanted.

Statement 162: A prosthetic mitral half valve for implanting into a native mitral valve comprising native leaflets connected to chordae tendinea, wherein the prosthetic mitral half valve is sized and configured to avoid interfering with the chordae tendinea.

Statement 163: A prosthetic mitral half valve for implanting into a native mitral valve comprising a native annulus and native leaflets connected to chordae tendinea, the prosthetic valve comprising a structural frame carrying a prosthetic posterior leaflet, wherein the structural frame extends no more than 20 mm from the mitral annulus into the left ventricle in areas where native chordae tendinea reside, when the prosthetic valve is implanted in the native valve.

Statement 164: The prosthetic valve of Statement 163 wherein the structural frame extends no more than 15, 10, or 8 mm from the mitral annulus into the left ventricle.

Statement 165: A prosthetic mitral half valve for implanting into a native mitral valve comprising a native annulus and native leaflets connected to chordae tendinea, the prosthetic valve comprising a structural frame with an annulus section configured to remain in apposition with the native annulus, and a ventricle section carrying a prosthetic posterior leaflet, the ventricle section extending no more than 20 mm from the annulus section.

Statement 166: The prosthetic valve of Statement 165 wherein the ventricle section extends no more than 15, 10, or 8 mm from the annulus section.

Statement 167: A prosthetic mitral half valve for implanting into a native mitral valve comprising a native annulus and native leaflets connected to chordae tendinea, the prosthetic valve comprising a chordae tendinea void defined in part by a structural frame, wherein the chordae tendinea void is configured to allow unencumbered passage of native chordae tendinea when the prosthetic valve is implanted.

Statement 168: A prosthetic mitral half valve for implanting into a native mitral valve comprising a posterior aspect and trigones, the prosthetic mitral half valve comprising a structural frame carrying a prosthetic posterior leaflet, and a first and second arm extending from the structural frame each comprising an anchor region, the first and second arms sized so that a compressive force is applied between the structural frame and a posterior aspect when the anchor regions are anchored to the trigones.

Statement 169: The prosthetic mitral half valve of Statement 168, wherein the first anchor region is configured to accept a first tissue anchor and aligned to pass the first tissue anchor into a first trigone, and the second anchor region is configured to accept a second tissue anchor and aligned to pass the second tissue anchor into a second trigone.

Statement 170: The prosthetic mitral half valve of Statement 169, wherein the first and second tissue anchors are each inserted from a left atrium, each respectively through the first and second anchor regions, then each respectively through the trigones.

Statement 171: The prosthetic mitral half valve of Statement 170, further comprising a third anchor region associated with the structural frame.

Statement 172: The prosthetic mitral half valve of Statement 171, wherein the third anchor region is configured to contact the posterior aspect of the native mitral valve.

Statement 173: The prosthetic mitral half valve of Statement 172, wherein the third anchor region is configured to contact an AV groove of the posterior aspect.

Statement 174: The prosthetic mitral half valve of Statement 172, wherein the third anchor region is on a leg extending from the structural frame.

Statement 175: The prosthetic mitral half valve of Statement 168, wherein the prosthetic posterior leaflet comprises a plurality of prosthetic scallops.

Statement 176: A prosthetic mitral half valve comprising a structural frame carrying prosthetic leaflets and arms extending from the structural frame, wherein the arms comprise anchoring regions and a bending moment in a plane substantially orthogonal to the structural frame.

Statement 177: A prosthetic mitral half valve for implanting into a native mitral valve comprising two trigones, the prosthetic mitral half valve comprising two trigone anchors at each end, wherein an arc length of the prosthetic mitral half valve spanning between the two trigone anchors is sized to be 95 to 110% of an arc length of the native mitral valve between the two trigones.

Statement 178: The prosthetic mitral half valve of Statement 177, comprising a prosthetic leaflet positioned between the trigone anchors.

Statement 179: The prosthetic mitral half valve of Statement 177, wherein the trigone anchors comprise anchor interfaces integrated with the prosthetic mitral half valve and separate piercing anchors configured to be connected to the anchor interfaces and the trigones.

Statement 180: The prosthetic mitral half valve of Statement 179, wherein the anchor interfaces are holes or channels.

Statement 181: A sizing tool for selecting a size of a prosthetic valve to be implanted in a native valve, comprising a shaft connected to a sizing face with a major axis, a minor axis, and circumference wherein the sizing tool has visual markers in regular increments along at least a portion of the major axis, the minor axis and circumference.

Statement 182: The sizing tool of Statement 181, wherein the sizing face is deployable from a contracted delivery state to a deployed state, wherein the delivery state is configured to slidably pass through a lumen having an inner diameter that is less than 24 French.

Statement 183: The sizing tool of Statement 182, wherein the sizing face is made from superelastic Nitinol.

Statement 184: A single leaflet mitral valve implant, comprising: a structural frame having a convex side as viewed in a transverse plane configured to conform to at least a portion of a native mitral valve annulus, and a concave side as viewed in a transverse plane configured to surround at least a portion of a flow path extending through the mitral valve implant; a single leaflet carried by the structural frame, configured to move between a first position in coaptation with a native anterior leaflet to obstruct the flow path, and a second position spaced laterally apart from the native leaflet to permit blood flow through flow path.

Statement 185: A single leaflet mitral valve implant as in Statement 184, further comprising a ventricular anchor carried by the structural frame.

Statement 186: A single leaflet mitral valve implant as in Statement 185, wherein the ventricular anchor is integrally formed with the frame.

Statement 187: A single leaflet mitral valve implant as in Statement 186, wherein the structural frame and ventricular anchor comprise Nitinol that is superelastic at body temperature.

Statement 188: A single leaflet mitral valve implant as in any preceding Statements 184 to 187, wherein the structural frame comprises a laser cut Nitinol sheet or tube, and the ventricular anchor comprises a formed Nitinol wire.

Statement 189: A single leaflet mitral valve implant as in Statement 185, wherein the ventricular anchor is configured to extend into the ventricle in the direction of the atrioventricular groove.

Statement 190: A single leaflet mitral valve implant as in Statement 189, wherein the frame further comprises at least a first atrial anchor.

Statement 191: A single leaflet mitral valve implant as in Statement 190, wherein the first atrial anchor comprises an arcuate support arm configured to extend circumferentially in a first direction around the flow path.

Statement 192: A single leaflet mitral valve implant as in Statement 191, further comprising a second atrial anchor comprising an arcuate support arm configured to extend circumferentially in a second direction around the flow path.

Statement 193: A single leaflet mitral valve implant as in Statement 192, wherein the first atrial anchor comprises a first free end spaced apart from a second free end of the second atrial anchor.

Statement 194: A single leaflet mitral valve implant as in Statement 192, wherein the first and second atrial anchors each comprise an anchor point configured to accept a tissue puncturing anchor.

Statement 195: A single leaflet mitral valve implant as in any preceding Statements 184 to 194, wherein the structural frame comprises a concave side as viewed in a sagittal plane configured to conform to at least a portion of the native mitral valve annulus, and a convex side as viewed in a sagittal plane configured to surround at least a portion of the flow path.

Statement 198: A single leaflet mitral valve implant as in Statement 195 in combination with Statement 65, wherein the ventricular anchor comprises a means for stabilizing the implant without puncturing native tissue.

Statement 199: A method of repairing a native mitral valve, comprising the steps of: Identifying a mitral valve having an annulus and a flow path between a native anterior leaflet and a defective native posterior leaflet; stabilizing the defective native posterior leaflet out of the flow path; and supporting a prosthetic posterior leaflet configured to move between a first position in coaptation with the native anterior leaflet to close the flow path and a second position spaced apart from the native anterior leaflet to open the flow path.

Statement 200: A method as in Statement 199, wherein the stabilizing is accomplished by positioning a structural frame of the prosthetic posterior leaflet along at least a portion of a first side and a second side of the native posterior leaflet to entrap the leaflet.

Statement 201: A method as in Statement 200, wherein the native posterior leaflet is entrapped at a position out of the flow path and spaced apart from an adjacent ventricular wall.

Statement 202: A method as in Statement 201, wherein the position out of the flow path and spaced apart from an adjacent ventricular wall comprises a distance in a range of 5 to 10 mm between the entrapped posterior leaflet and the ventricular wall during ventricular systole.

Statement 203: A method as in Statement 199, wherein the supporting step comprises positioning a frame adjacent the annulus, the frame having at least one ventricular anchor, at least one atrial anchor, and the prosthetic posterior leaflet.

Statement 204: A method as in Statement 203, wherein the positioning step comprises transvascular advance of a deployment catheter to the vicinity of the mitral valve, and deploying the frame from the deployment catheter.

Statement 205: A method as in Statement 204, wherein the at least one ventricular anchor comprises a single ventricular anchor positioned along a center line of symmetry of the frame.

Statement 206: A method as in Statement 204 or 205, wherein the ventricular anchor is adapted to do the entrapping step and engage with an atrioventricular groove of the native mitral valve.

Statement 207: A method as in Statement 203, wherein the at least one atrial anchor comprises no more than two atrial anchors, and wherein the two atrial anchors each comprise an anchor point, and wherein the stabilizing step comprises inserting a tissue piercing anchor through each of the anchor points and into native tissue comprising a trigone or tissue within 3 mm of the trigone.

Statement 208: An implant for mitral valve repair, comprising: a support frame configured to conform to at least a portion of a mitral valve annulus, the support frame expandable from a first, reduced crossing profile to a second, enlarged implanted profile; a single leaflet carried by the frame, configured to move between a first position in coaptation with a native anterior leaflet to obstruct the flow path, and a second position spaced laterally apart from the native anterior leaflet to permit blood flow through the flow path, when the frame is in the enlarged, implanted profile.

Statement 209: A partial mitral valve implant as in Statement 208, further comprising a ventricular anchor carried by the support frame.

Statement 210: A partial mitral valve implant as in Statement 209, wherein the ventricular anchor is integrally formed with the frame.

Statement 211: A partial mitral valve implant as in Statement 210, wherein the frame and ventricular anchor comprise Nitinol.

Statement 212: A partial mitral valve implant as in Statement 209, wherein the ventricular anchor is configured to extend into the ventricle in the direction of the atrioventricular groove.

Statement 213: A partial mitral valve implant as in Statement 212, wherein the frame further comprises at least a first atrial anchor.

Statement 214: A partial mitral valve implant as in Statement 213, wherein the first atrial anchor comprises an arcuate support arm configured to extend circumferentially in a first direction around the flow path.

Statement 215: A partial mitral valve implant as in Statement 214, further comprising a second atrial anchor comprising an arcuate support arm configured to extend circumferentially in a second direction around the flow path.

Statement 216: A partial mitral valve implant as in Statement 215, wherein the first atrial anchor comprises a first free end spaced apart from a second free end of the second atrial anchor.

Statement 217: A partial mitral valve implant as in Statement 215, wherein the first arcuate support arm and the second arcuate support arm are each connected to the support frame at a first end and to one another at a second end.

Statement 218: A partial mitral valve implant as in Statement 208, wherein the second position is paced laterally from a center of the flow path.

Statement 219: A partial mitral valve implant as in Statement 208, wherein the flow path comprising the partial mitral valve implant has a cross sectional area in a transverse plane that is at least 90% of the cross sectional area of a flow path without the partial mitral valve implant.

Statement 220: A partial mitral valve implant as in Statement 219, wherein the at least 90% comprises at least 95%.

Statement 221: A partial mitral valve implant as in Statement 219, wherein the at least 90% comprises 100%.

Statement 222: A mitral valve repair system, comprising: a delivery catheter, comprising an elongate, flexible tubular body, having a proximal end, a distal end and a central lumen; an implant within the central lumen, the implant comprising a frame and a single leaflet, the frame expandable between a compressed configuration within the lumen, and an expanded configuration when deployed out of the lumen.

Statement 223: A mitral valve repair system as in Statement 222, further comprising an actuator, axially movably carried within the lumen, for deploying the implant from the lumen.

Statement 224: A mitral valve repair system as in Statement 223, further comprising at least one tissue anchor configured to attach the frame within a mitral valve annulus.

Statement 225: A mitral valve repair system as in Statement 224, wherein the at least one tissue anchor comprises no more than two tissue anchors.

Statement 226: A mitral valve repair system as in Statement 225, further comprising an anchor driver, configured to rotate the tissue anchor.

Statement 227: A mitral valve repair system as in Statement 222, wherein the frame is self-expandable.

Statement 228: A mitral valve repair system as in Statement 222, wherein the frame is balloon-expandable.

Statement 229: An implant for repairing a mitral valve, comprising: A self-expandable arcuate frame configured for implantation within a mitral valve annulus having a first commissure spaced apart from a second commissure by an annulus major arc length; wherein the frame has a frame arc length that is no more than about 90% of the annulus arc length (e.g., in a range of 40% to 75%, in a range of 50% to 75%).

Statement 230: An implant as in Statement 229, further comprising first and second atrial anchoring arms extending in opposite directions from the frame.

Statement 231: An implant as in Statement 230 wherein the frame arc length resides on a plane, and the first and second atrial anchoring arms reside in the same plane.

Statement 232: An implant as in Statement 231, further comprising at least one ventricular anchor.

Statement 233: An implant as in Statement 232, wherein the at least one ventricular anchor extends transverse to the plane.

Statement 234: An implant as in Statement 233, wherein the at least one ventricular anchor, comprises a single anchor connected to the frame and centered in the frame arc length.

Statement 235: A single leaflet implant, comprising: an expandable frame having a main body and left and right atrial anchoring arms extending in opposite directions from the main body, the main body and left and right arms curved about a flow path; and a ventricular anchor extending in a first direction along the flow path; and a single leaflet carried by the frame.

Statement 236: A single leaflet implant as in Statement 235, wherein the ventricular anchor extends in the first direction to a bend from which it extends in a second direction angularly displaced from the first direction.

Statement 237: A single leaflet implant as in Statement 236, wherein the atrial anchoring arms are configured for implantation adjacent and superior to a mitral valve annulus and the ventricular anchor is configured to extend into the ventricle and immobilize a native posterior leaflet.

Statement 238: A method of in situ construction of a hemi prosthetic mitral valve, comprising the steps of: providing an implant having an expandable frame with a single leaflet supported by an arcuate main body, left and right atrial anchoring arms extending in opposite directions from the main body, the main body and left and right atrial anchoring arms curved about a flow path, and a ventricular anchor extending along the flow path; attaching the left and right anchoring arms to an atrial side of a mitral valve annulus; and attaching the ventricular anchor in the ventricle to stabilize the leaflet such that it can move into and out of coaptation with a native anterior leaflet.

Statement 239: A method of in situ construction of a hemi prosthetic mitral valve as in Statement 238, additionally comprising the step of entrapping a portion of a native leaflet with the ventricular anchor.

Statement 240: A method of in situ construction of a hemi prosthetic mitral valve as in Statement 239, wherein the portion of a native leaflet comprises a native P2 scallop of a native posterior leaflet.

Statement 241: A method of in situ construction of a hemi prosthetic mitral valve as in Statement 240, wherein the entrapping step comprises stabilizing the native P2 scallop in a position spaced apart from a ventricular wall.

Statement 242: A method of in situ construction of a hemi prosthetic mitral valve as in Statement 238, wherein the attaching steps are accomplished in an open surgical procedure.

Statement 243: A method of in situ construction of a hemi prosthetic mitral valve as in Statement 238, wherein the attaching steps are accomplished in a transvascular procedure.

Statement 244: A method of in situ construction of a hemi prosthetic mitral valve as in Statement 243, wherein the transvascular procedure is accomplished via a femoral access.

Statement 245: A method of in situ construction of a hemi prosthetic mitral valve as in Statement 243, wherein the implant is delivered from a delivery catheter lumen having a diameter of no more than about 28 F (e.g., no more than 27 F, no more than 26 F, no more than 25 F, no more than 24 F, no more than 23 F, no more than 22 F, no more than 21 F, no more than 20 F).

Statement 246: A method of in situ construction of a hemi prosthetic mitral valve as in Statement 238, comprising inserting no more than two tissue piercing anchors.

### BRIEF DESCRIPTION OF DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the drawings, can be arranged, substituted, combined, and designed in a wide variety of different configurations, all of which are explicitly contemplated and make part of this disclosure.

FIG. 1 is a schematic illustration of a superior perspective of a transverse cross section of a heart showing a native mitral valve.

FIG. 2 is a schematic illustration of an anterior perspective of a coronal cross section of a heart.

FIG. 3A is an illustration of a structural frame laser-cut from a flat sheet of Nitinol.

FIG. 3B is an illustration of another design of a structural frame laser-cut from a flat sheet of Nitinol.

FIG. 4A is an illustration of a posterior perspective of a shape-set structural frame of FIG. 3A.

FIG. 4B is an illustration of a medial perspective of a shape-set structural frame of FIG. 3A.

FIG. 4C is an illustration of a superior perspective of a shape-set structural frame of FIG. 3A.

FIG. 4D is an illustration of a posterior perspective of a shape-set structural frame of FIG. 3B.

FIG. 5A is an illustration of a posterior perspective of a structural frame with an anchoring leg connected to the frame.

FIG. 5B is a schematic illustration of a medial perspective of a structural frame with an anchoring leg.

FIG. 5C is a schematic illustration of a medial perspective of another design of a structural frame with an anchoring leg.

FIG. 6A is an illustration of a 2-dimensional leaflet pattern.

FIG. 6B is an illustration of another design of a 2-dimensional leaflet pattern

FIG. 7A is an illustration of a superior perspective of a prosthetic mitral valve.

FIG. 7B is an illustration of an anterior perspective of a prosthetic mitral valve.

FIG. 7C is an illustration of an inferior perspective of a prosthetic mitral valve.

FIG. 7D is an illustration of an isometric perspective of a prosthetic mitral valve.

FIG. 8A is a schematic illustration of a superior perspective of a transverse cross-section of a heart showing a native mitral valve with a prosthetic valve implanted, wherein a membrane and fabric layer are omitted for clarity.

FIG. 8B is a schematic illustration of a medial perspective of a coronal cross-section of a heart showing a native mitral valve with a prosthetic valve implanted, wherein a membrane, fabric layer, and P1 and P3 scallops are omitted for clarity.

FIG. 8C is an illustration of a posterior perspective of another design of a structural frame with an anchoring leg, wherein a membrane, fabric layer, arms, and P1 and P3 scallops are omitted for clarity.

FIG. 8D is a schematic illustration of a superior perspective of a transverse cross-section of a heart showing a native mitral valve with a prosthetic valve implanted, wherein a membrane and fabric layer are omitted for clarity and an anterior arm bridge is shown.

FIGS. 9A, 9B and 9C are schematic illustrations of a tissue anchor being inserted and deployed.

FIGS. 10A and 10B are schematic illustrations of a cross-section of a tissue anchor and deployment device.

FIGS. 11A and 11B are schematic illustrations of another design of a cross-section of a tissue anchor and deployment device.

FIG. 12A is a schematic illustration of a step of deployment of a prosthetic valve.

FIG. 12B is a schematic illustration of a step of deployment of a prosthetic valve.

FIG. 12C is a schematic illustration of a step of deployment of a prosthetic valve and a design with a tissue anchor placed through an atrial section, posterior annulus, and anchoring leg.

FIGS. 13A to 13D show various stages of deployment of a prosthetic valve from a delivery system.

FIG. 14 shows a prosthetic valve having secondary stabilization legs

FIGS. 15A to 15C show stages of manipulation of a prosthetic valve during implantation.

FIGS. 16A to 16F show stages of delivery of a tissue anchor.

FIGS. 16G to 16H show alternative examples of tissue anchors

FIG. 16I is a schematic illustration of a prosthetic valve being implanted in a patient's heart.

FIG. 16J is a flow chart showing the steps of the stages from FIGS. 16A to 16F.

FIGS. 17A to 17C show embodiments of prosthetic valves within situ adjustable tensioners.

FIG. 18A is a schematic illustration of a simplified 2-D representation of a bottom view of a prosthetic valve with a leaflet scallop in a closed and open state.

FIG. 18B is a schematic illustration of a bottom view of a prosthetic valve with a leaflet scallop in a closed and open state.

FIG. 18C is a simplified 2-D representation of the arrangement in FIG. 18A showing volume of the scallop in the closed valve state 265A and volume of the space into which the scallop can move into.

FIG. 18D is a simplified 2-D representation of an alternative design having improved space for a scallop to move into in its open state.

FIG. 18E is a 2-D simplified representation of the concept shown in FIG. 18D showing the three scallops P1, P2, P3 of the prosthetic posterior mitral leaflet.

FIG. 18F is a schematic illustration of a bottom view of an example of a prosthetic valve corresponding to the simplified version in FIG. 18E.

FIG. 18G is a 2-D simplified representation of a prosthetic mitral valve wherein the commissures are elastically movable from a closed valve position to an open valve position.

FIG. 18H is a 2-D simplified representation of a prosthetic mitral valve wherein the commissures are positioned within the flow path and the ventricle section of the frame is exterior to the flow path in an arrangement that allows the prosthetic scallop to fully open.

FIG. 18I is a 2-D simplified representation of a prosthetic mitral valve wherein the commissures are positioned out of the flow path and the ventricle section of the frame is exterior to the flow path in an arrangement that allows the prosthetic scallop to fully open and further optimize flow.

FIG. 18J is a schematic illustration of a bottom view of an alternative example of a prosthetic valve wherein blood filling each scallop is distributed more evenly among each scallop.

FIG. 19A is a schematic illustration of a top view of an example of a prosthetic valve corresponding to the simplified version in FIG. 18G.

FIG. 19B is a schematic illustration of a side view of an example of a prosthetic valve corresponding to the simplified version in FIG. 18G.

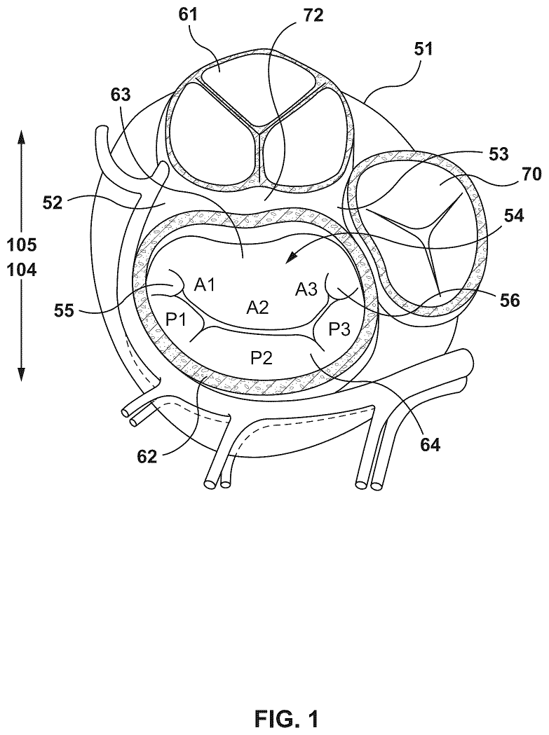

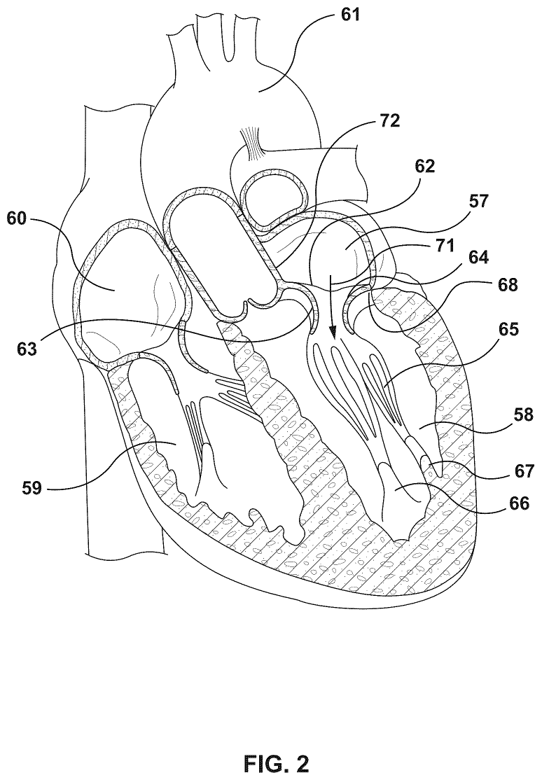

### DETAILED DESCRIPTION OF THE INVENTION

The disclosure herein is related to prosthetic hemi-mitral valves and delivery systems. The following detailed description is directed to certain specific embodiments. In this description, reference is made to the drawings wherein like parts or steps may be designated with like numerals throughout for clarity. Reference in this specification to “one embodiment,” “an embodiment,” or “in some embodiments” means that a particular feature, structure, or characteristic described in connection with the embodiment is included in at least one embodiment of the invention. The appearances of the phrases “one embodiment,” “an embodiment,” or “in some embodiments” in various places in the specification are not necessarily all referring to the same embodiment, nor are separate or alternative embodiments necessarily mutually exclusive of other embodiments. Moreover, various features are described which may be exhibited by some embodiments and not by others. Similarly, various requirements are described which may be requirements for some embodiments but may not be requirements for other embodiments. Reference will now be made in detail to embodiments of the invention, examples of which are illustrated in the accompanying drawings).

Disclosed herein is a prosthetic posterior mitral leaflet, which may provide a solution to a large percentage of patients suffering from functional or degenerative mitral valve regurgitation. Some implementations of the prosthetic valve may be implanted surgically or some, preferably with a transvascular approach (e.g., transfemoral, transaxillary, transradial, transjugular entry), which may include delivery through a lumen that is 28 FR or less (e.g., 27 FR or less, 26 FR or less, 25 FR or less, 24 FR or less, 23 FR or less, 22 FR or less, 21 FR or less, 20 FR or less, 19 FR or less). Compared to many of the full valve replacements, which require implanting a frame with valves into a structural frame, the prosthetic posterior mitral valve may be made up of significantly less material (e.g., about 1/4 the amount of material), which may have beneficial impacts on function, compacted delivery size, and ease of implanting. The prosthetic valve encapsulates the faulty posterior mitral leaflet while restoring as much native valve function as possible, including preservation of the anterior leaflet, a maximum GOA, minimal disruption of the chordae and papillary muscles, optimal rinse to avoid thrombosis, and complete seal through the mitral valve or around the prosthetic. The posterior prosthetic valve may reduce or eliminate impingement on the adjacent aortic valve, thus reducing or eliminating LVOT obstruction. The prosthetic valve may be configured to preserve future interventions in the case of further heart failure. For example, the prosthetic valve may accommodate or permit insertion of a different prosthetic valve (e.g., a valve in a valve).

The prosthetic valve has a structural frame that conforms at least in part to the posterior atrium and posterior annulus, holds back the native posterior leaflet, and provides a framework to hold a prosthetic posterior leaflet, which may comprise one or a plurality of leaflets (e.g., 2, 3, 4, 5), preferably 3. A ventricular anchoring leg, or a plurality of legs, hooks behind the frame between chordae and into the AV groove 68 preventing the prosthetic valve from dislodging up into the atrium when the left ventricle is pressurized. The prosthetic posterior leaflet(s) billow when the left ventricle is pressurized causing a strong coaptation connection with the anterior leaflet and may further apply force to the posterior aspect of the prosthetic valve to further secure engagement with the heart. Atrial anchoring arms (e.g., two arms) are anchored to tissue in the atrium (e.g., into trigones) and apply suitable forces to the structural frame for stability and proper functioning of the valve. The prosthetic valve may have additional anchoring or stabilizing features that contribute to maintaining correct position with minimal interruption of native structures.

For the purpose of discussion orientation of the components may be described in terms of a superior direction 102 which is intended to be implanted in a patient's heart toward the superior aspect of the patient (i.e., cranially, or towards the head); an inferior direction 103, which is intended to be implanted in the patient's heart toward the inferior aspect of the patient (i.e. caudally, or towards the feet); a posterior direction 104, which is intended to be implanted toward to the patient's back; and an anterior direction 105, which is intended to be implanted toward the patient's front.

### Structural Frame

A structural frame functions to provide a skeletal structure to hold other parts together so they may be positioned properly with respect to one another and to native structures, and forces are applied to the parts and native structures appropriately for proper functioning.

The frame may be deployable from a condensed delivery configuration to an expanded implant configuration and in its expanded configuration have a crescent shape, optionally with two free ends that does not complete a full closed cylinder (e.g., having no free ends). The delivery configuration may be adapted for passage of the prosthetic valve through a transvascular delivery catheter having a size of about 28 French or less (e.g., 27 FR or less, 26 FR or less, 25 FR or less, 24 FR or less, 23 FR or less, 22 FR or less, 21 FR or less, 20 FR or less, 19 FR or less). A posterior valve without an anterior replacement reduces volume and allows passage through a smaller catheter compared to a whole valve replacement. Superelastic Nitinol (e.g., laser-cut sheet, laser-cut tube, or alternatively braided or woven structure, or wire-formed structure) allows a high degree of compression without permanent deformation so the frame can deploy to the implanted configuration. A partial valve replacement having a crescent-shaped frame can move with the mitral annulus with minimal impeding of natural annulus movement. This design may avoid the need to have a separate anchoring frame as seen in some prior art, and the frame may function for both anchoring and holding the leaflets.

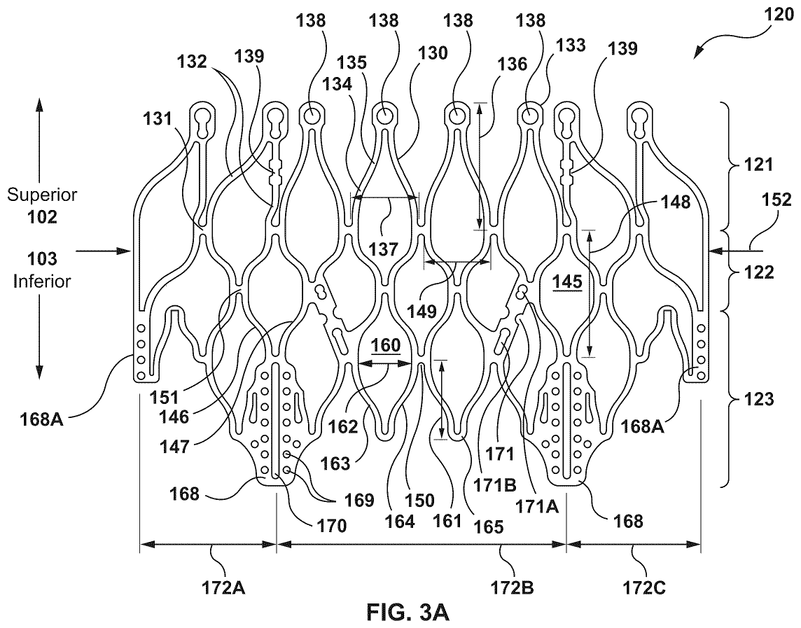

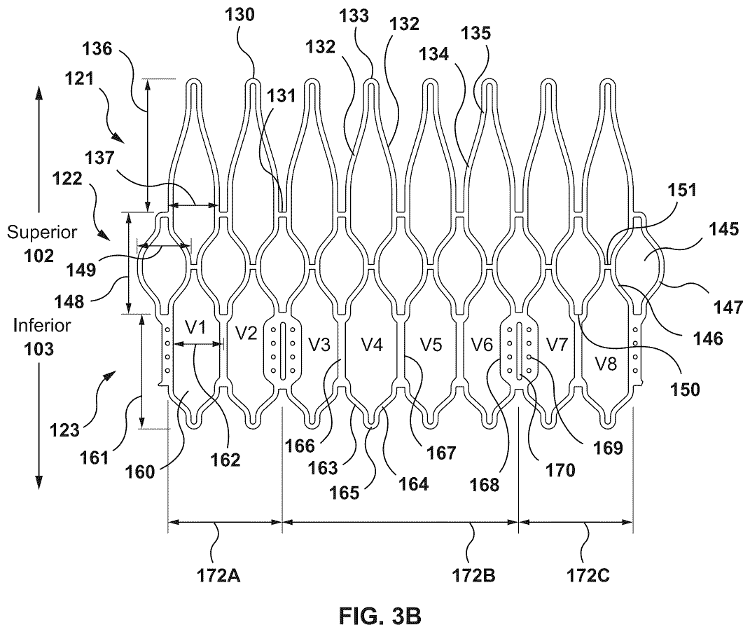

The frame may be made by laser cutting a thin sheet or tube of superelastic Nitinol, as shown in FIG. 3A or an alternative design shown in FIG. 3B, which shows a frame before shape-setting, cut from a flat sheet. The laser-cut frame may be electropolished.

Dimensions of the structural frame may vary to provide multiple prosthetic valves that can be used for various patient sizes.

The frame may have an atrial section 121 located on the superior aspect of the frame, a ventricle section 123 located on the inferior aspect of the frame, and an annulus section 122 located between the atrial and ventricle sections.

The atrial section 121 may have a plurality of atrial petals 130 (e.g., eight), each having two atrial petal struts 132 connected to one another by an atrial petal apex 133. The atrial petal apex 133 may have a tight curve which may function to provide a flexible and atraumatic interaction with native tissue and ability to transition from a delivery to implant configuration. Each atrial petal may have an atrial petal length 136, which may be in a range of 6 to 15 mm, and an atrial petal width 137, which may be in a range of 3 to 10 mm. Each atrial petal may have the same dimensions, as shown in FIG. 3B, or have differences as shown in FIG. 3A, for example wherein the atrial petals on the ends are angled toward the center of the frame. The atrial petal struts 132 may have a curvature as shown, which may include an S-shaped curve having a first inflection 134 and a second inflection 135. The atrial petals may be connected to one another at an inferior aspect of the atrial petal struts 132 by a superior annulus cell strut 131, which may also form or connect the atrial petals to the annulus section 122. The atrial petals may be tapered in the superior direction (i.e., wherein the atrial petal struts 132 are further apart or equal to the atrial petal width 137 at the inferior end and closer together or equal to the atrial petal apex diameter of curvature at the superior end). Atrial petal cells are spaces defined by two atrial petal struts 132 connected by an atrial petal apex 133, and one or two annulus cell struts 146, 147. An inferior portion of the atrial petal cells may extend from the atrial section 121 to the annulus section 122.

An atrial section 121 may further comprise delivery interface holes 138, and arm attachment features 139, as shown in FIG. 3A.

Delivery interface holes 138 are structures for temporarily connecting the prosthetic valve to a delivery tool. The delivery interface holes 138 may be located at the atrial petal apexes 133 and may be on each atrial petal apex or at least one (e.g., two, three, four, five, six, seven, eight).

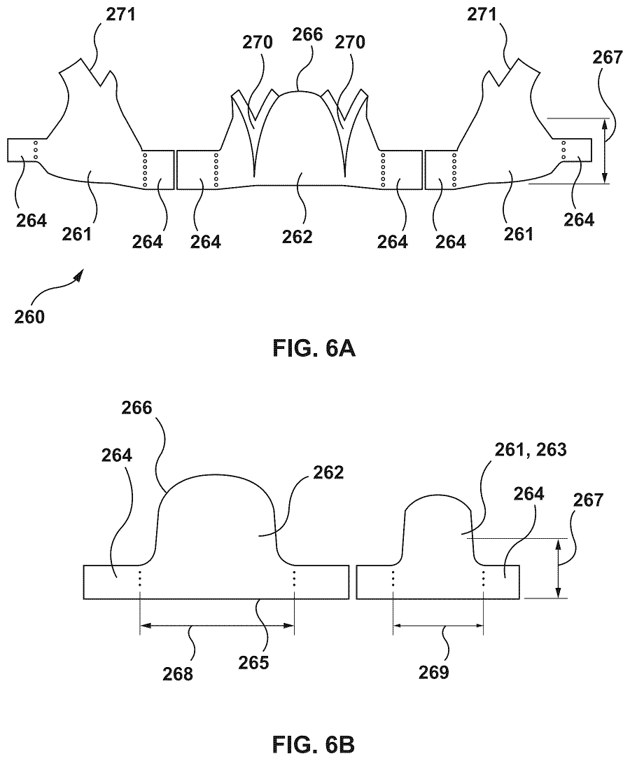

Arm attachment features 139 may be arranged with horizontally and vertically spaced placement of the attachments to provide a secure connection of the base of the arms so the arms are cantilevered to the structural frame. Arm attachment features 139 may be features that allow securement of the arms to the frame such as protrusions (e.g., two), as shown, that a suture may be tied between, holes that sutures may be threaded through, or holes that an arm wire may be inserted through. The arms may be made of shape-set superelastic Nitinol wire and be connected to the arm suture attachments 139 of the frame as shown in FIG. 6A, FIG. 6B, and FIG. 6D.

The annulus section 122 may have a plurality of annulus cells 145 (e.g., seven as shown in FIG. 3A, nine as shown in FIG. 3B), each being spaces defined by a first annulus cell strut 146 and a second annulus cell strut 147. Each annulus cell 145 may have an annulus cell length 148, which may be in a range of 6 to 15 mm, and an annulus cell width 149, which may be in a range of 2 to 12 mm. The annulus cell length 148 may be adapted to span the height of a patient's mitral valve annulus 62. The total width (e.g., annulus section width 152 shown in FIG. 3A) of the structural frame 120 may be adapted to span the arc length of a portion (e.g., less than 100%, in a range of 70 to 85%) of the patient's posterior annulus, for example to preserve a portion (e.g., 10% to 50%) of each of the native P1 and P3 leaflets. Alternatively, the total width of the structural frame 120 may be equal to the annulus cell width 149 and middle annulus cell strut 151 multiplied by the quantity of annulus cells 145 and may be adapted to span the arc length of the patient's posterior annulus or a portion thereof, which may be defined as the portion of the mitral annulus containing the native posterior leaflet from the anterolateral commissure 55 to the posteromedial commissure 56 (e.g., in a range of 40 to 95 mm, about 80 mm). The annulus cell width 149 may be equal to the atrial petal width 137. The first annulus cell strut 146 may be connected to the second annulus cell strut 147 at their superior ends by a superior annulus cell strut 131 and at their inferior ends by an inferior annulus cell strut 150. Both the superior annulus cell strut 131 and inferior annulus cell strut 150 may have the same width. Each annulus cell 145 may be connected to an adjacent annulus cell by a middle annulus cell strut 151, which may have width equal to the superior 131 and inferior 150 annulus cell struts. The middle annulus cell strut 151 may connect a second annulus cell strut 147 of an annulus cell 145 to a first annulus cell strut 146 of an adjacent annulus cell 145.

The ventricle section 123 may have a plurality of ventricle cells 160 (e.g., six as shown in FIG. 3A, eight as shown in FIG. 3B, or a quantity equal to the quantity of atrial petals), each being spaces defined by a first ventricle cell strut 163 and a second ventricle cell strut 164, which join one another at an inferior ventricle cell apex 165, a first ventricle cell vertical strut 166, a second ventricle cell vertical strut 167, and the first annulus cell strut 146 and second annulus cell strut 147 of an adjacent annulus cell 145. The ventricle cells 160 may extend into the annulus section 123. Adjacent ventricle cells 160 may share an adjoining ventricle cell vertical strut 166, 167. Some of the ventricle cell vertical struts may include a leaflet connection frame 168 for attaching a prosthetic leaflet. A leaflet connection frame 168 may have a plurality of leaflet eyelets 169 for passage of sutures for sewing a prosthetic leaflet to. Leaflet connection frames 168 that are between two adjacent ventricle cells 160 (e.g., between the V2 and V3, or the V6 and V7 ventricle cells as shown in FIG. 3B) may have a leaflet slot 170 for passage of a portion of a leaflet through the structural frame from an inner side to and outer side. Leaflet connection frames 168 that are on the lateral sides of the frame and connected to only one ventricle cell (e.g., connected to the V1 or V8 ventricle cells as shown) may be absent a leaflet slot. Each ventricle cell 160 may have a ventricle cell length 161, which may be in a range of 10 to 18 mm, and a ventricle cell width 162, which may be in a range of 3 to 5 mm and may be equal to the atrial petal width 137. The ventricle cell length 161 may be adapted to span at least (e.g., equal to, or longer, longer by a range of 8 to 15 mm) the height of the patient's native posterior mitral valve leaflet.

The ventricle section 123 may have leaflet connection frames to hold at least one prosthetic posterior leaflet (e.g., 1, 2, 3, 4, or 5 leaflets), preferably three as shown in FIG. 3A or 3B (or shown with leaflets connected in FIG. 6A) to mimic the native P1, P2 and P3 posterior leaflets. The width 172B between the P2 leaflet connection frames may be larger than (e.g., twice as wide as) the width 172A between the P1 leaflet connection frames or the width 172C between the P3 leaflet connection.

As shown in FIG. 3A, a structural frame 120 may have a ventricle section 123 with a shorter ventricular height 161 compared to FIG. 3B, leaflet connection frames that are raised on the sides, or leg suture attachments 171. The shorter height of the ventricle section 161, e.g., in a range of 6 to 14 mm, functions to hold the leaflets while avoiding impingement upon chordae tendinea, and thus papillary muscles and ability of prosthetic valve to sit securely and close properly. Optionally, struts that form ventricle petals, may extend inferiorly no further than leaflet connection frames 168 for the P2 leaflet so that there is minimal frame material extending caudally into the ventricle to leave as much room as possible for native chordae to remain unimpinged.

The leaflet connection frames 168A on the sides of the structural frame 120 may be shifted in a superior direction relative to the leaflet connection frames 168. This may allow the P1 and P3 prosthetic leaflets to have an angled free edge, which may improve coaptation, and leave space for native chordae.

Leg attachment features 171, 171A, 171B provide a secure connection of the leg 190 to the frame 120. For example, leg attachments 171A may be spaced from leg attachments 171B with a vertical distance in a range of 2 to 10 mm (e.g., 4 mm) and horizontal distance in a range of 0 to 5 mm, and may have indents, holes, protrusions or other features that prevent a suture from sliding on the frame, which functions to hold the leg securely to the frame and prevent it from sliding or rotation with respect to the frame. As shown in FIG. 3A a leg attachment feature for connecting one of two ends of a leg may include a first hole 171A, a second hole 171B, and a suture indent 171. A Nitinol wire-formed anchoring leg may have a base wire that is passed through the first hole 171A and through the second hole 171B and a suture may be tied around the wire and held in place by the indents 171. The leg may have a second base wire connected to the frame in the same way on the other side.

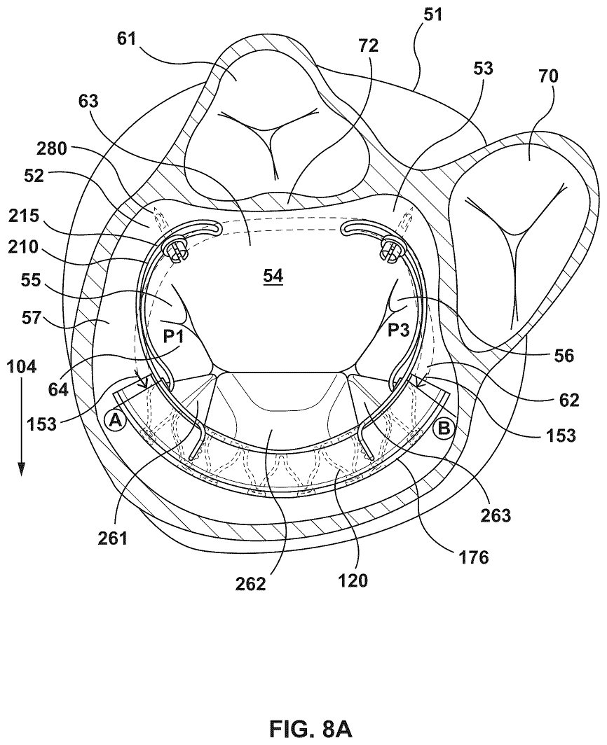

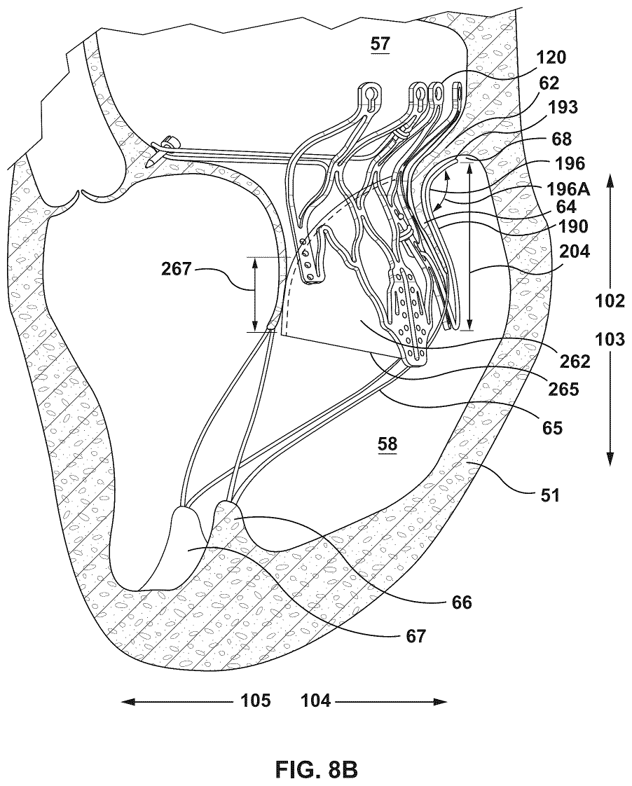

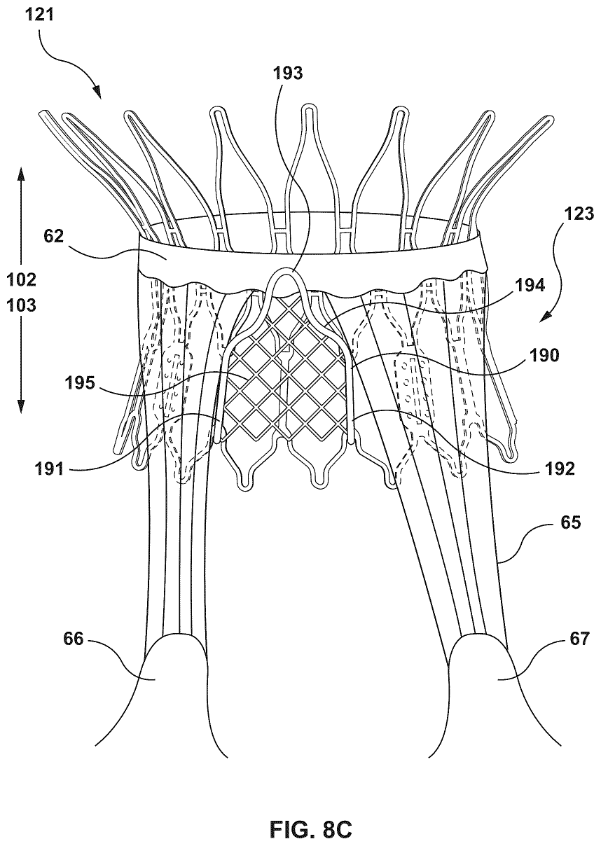

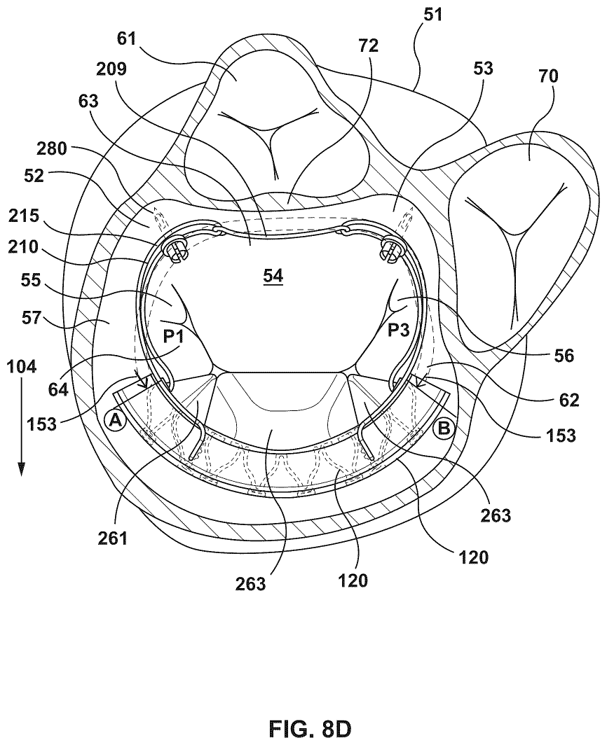

In the flat configuration of the frame 120 the width, for example the annulus section width 152 as shown in FIG. 3A, may be configured so that when the frame 120 is shape set the annulus section width 152 corresponds with an arc length 153 (FIG. 8D) chosen to match a predetermined percentage of an arc length between the commissures of the native mitral annulus, for example the predetermined percentage may be in a range of 30 to 100% (e.g., 30 to 80%, 40 to 80%, 50 to 70%).

Alternatively, a structural frame made be made with one or more legs and arms integrated as one piece, for example they may be laser cut from one piece of Nitinol, which could save money and manufacturing time.

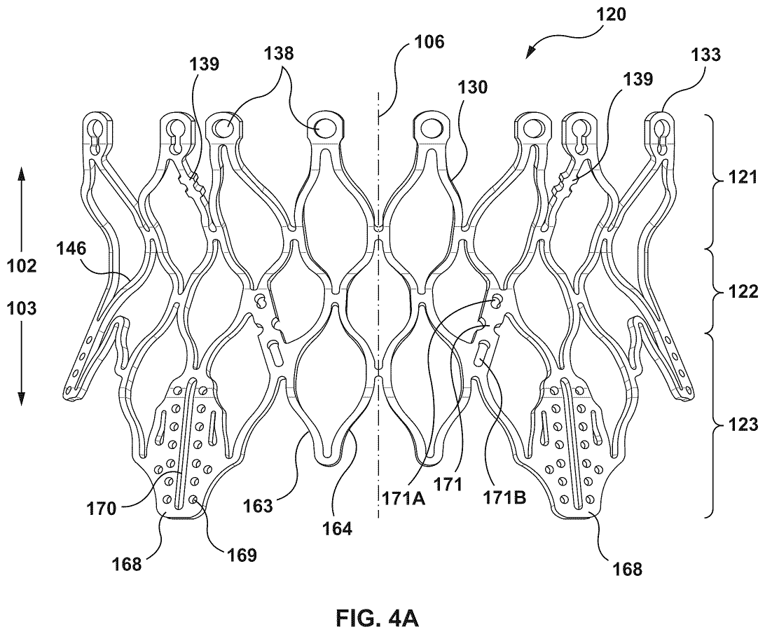

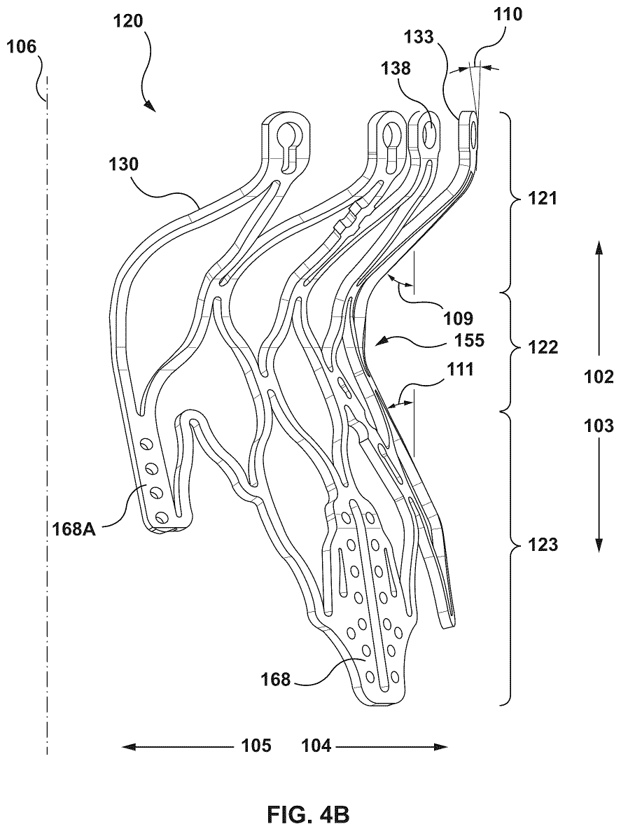

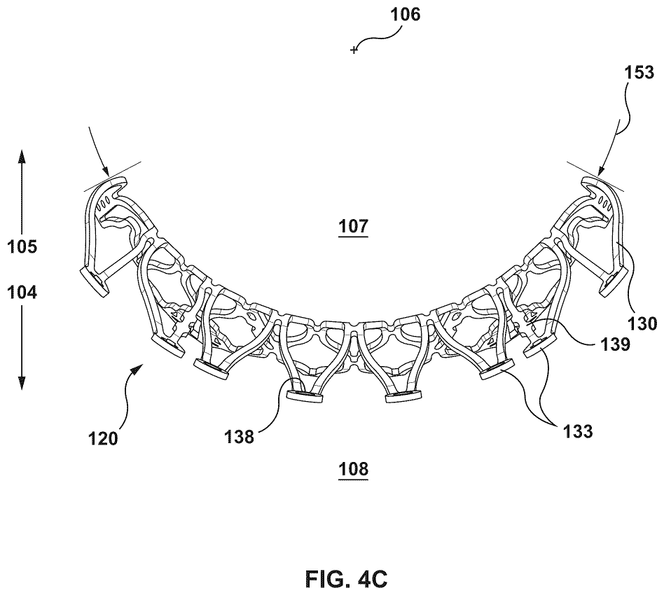

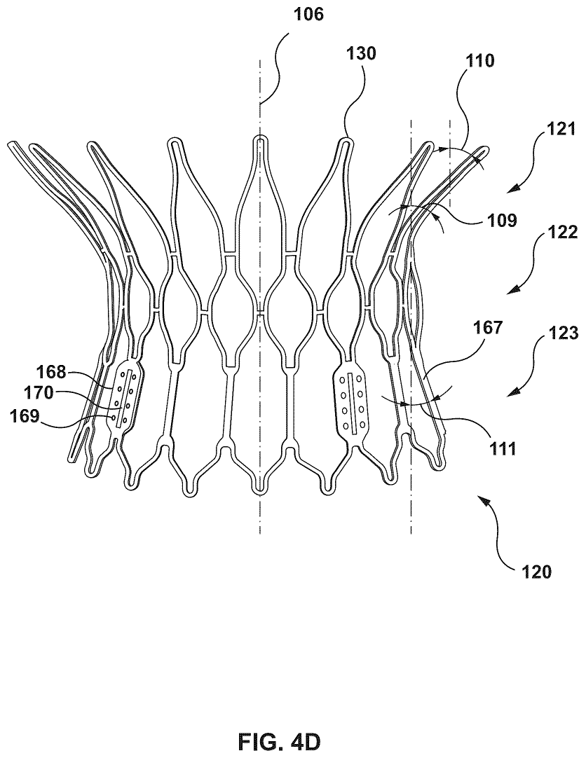

The structural frame 120 of FIG. 3A is shown in a shape-set configuration in a posterior view in FIG. 4A, a medial view in FIG. 4B, and a superior view in FIG. 4C, wherein the laser-cut frame is held in a complex three-dimensional shape by a shape-setting mold and heated to program the shape using methods known in the field of shape-setting superelastic Nitinol. The structural frame 120 of FIG. 3B is shown in a shape-set configuration in FIG. 4D. In FIGS. 4A to 4D only the structural frame is shown. The shape-set configuration gives the prosthetic valve its unconstrainted shape, wherein external forces are not applied by a delivery catheter or native tissues. When the prosthetic valve is released from a delivery catheter into the heart the structural frame 120 expands due to the superelastic Nitinol properties from a constrained delivery configuration toward the shape-set configuration until an equilibrium of forces is reached when contacting native tissue and the deployed implant configuration is reached. From a top view, or view from the native left atrium as shown in FIG. 4C, the shaped structural frame is curved in the transverse plane to mimic the curvature of the native posterior mitral valve structures, having an internal side 107, which is the side facing the mitral valve opening, and an external side 108, which is the side facing the posterior mitral annulus 62. The curvature may be somewhat elliptical or oval about a central axis 106.

Arc length 153 of the structural frame 120 in a transverse plane (FIG. 4C) may be equal to the annulus section width 152 of the frame (FIG. 3A). Arc length 153, and optionally other dimensions such as arm length, leaflet dimensions, or ventricular cell length, of a prosthetic valve may be selected based on native valve circumference or major axis or minor axis, a sizing tool, position of or distance between native commissures, position of or distance between native leaflets, position of or distance between native trigones. The arc length 153 of the prosthetic valve may be shorter than the major arc between native commissures 55, 56, for example shorter by an amount in a range of 20 to 40 mm (e.g., 25 to 35 mm, 28 to 32 mm) (see FIG. 8A). For example, the arc length of the structural frame from point A to B (see FIG. 8A, 8D) relative to the native mitral valve may be such that the prosthetic valve covers the P2 and only a portion of P1 and P3, for example, a portion in a range of 30 to 100% (e.g., 30 to 40%, 40 to 60%, 50 to 70%). Another example of a way to characterize partial replacement of the posterior leaflet is to say the arc length of the structural frame from point A to B (see FIG. 8A, 8D) is sized to be in a range of 30% to 40% of the circumference of the native mitral valve annulus during ventricular diastole. Another example of a way to characterize partial replacement of the posterior leaflet is to say the arc angle of the arc length of the structural frame from point A to B (see FIG. 8A, 8D) is sized to span a range of 30% to 40% of the native mitral valve orifice on a transverse plane during ventricular diastole. This may allow the uncovered portions of the P1 and P3 leaflets to continue to function as valve components, which has shown in studies to seal well against the native anterior leaflet 63 and prosthetic P1 and P3 leaflets respectively. Furthermore, the arc length 153 with respect to the native valve may function to avoid interruption of the native anterior leaflet 63 and chordae tendinea 65, improve coaptation, prevent valvular leakage, prevent perivalvular leakage, ensure secure sitting, allow containment of the native posterior leaflets, and allow for greater tolerance when sizing a prosthetic to a native valve for example, the prosthetic valve may be selected to fit in a native valve wherein the prosthetic valve has an arc length within a range of 30 to 90% (e.g., 30 to 80%, 40 to 80%, 50 to 70%) of the major arc length between the native commissures). This arc length and partial replacement of the native P1 and P2 scallops may also provide a larger tolerance in rotational alignment in the transverse plane, compared to a design requiring precise alignment of the edges with the commissures. For example, a method of implanting the prosthetic valve may include finding a center line of the native mitral valve in the transverse plane and rotationally aligning the prosthetic valve so that the center line of the prosthetic valve is within +/−25 degrees of the center line of the native mitral valve and deploying anchors to secure the prosthetic valve in said rotational alignment; or a method of implanting the prosthetic valve may include rotationally aligning the prosthetic valve so that the prosthetic P1 scallop replaces a portion of the native P1 scallop in a first range of 30 to 90% (e.g., 30 to 80%, 40 to 80%, 50 to 70%) and the prosthetic P2 scallop replaces a portion of the native P2 scallop in a second range of 30 to 90% (e.g., 30 to 80%, 40 to 80%, 50 to 70%), wherein the first range and the second range are not equal; or a method of implanting the prosthetic valve may include selecting a prosthetic valve from a group of valves of varying sizes, based on the arc length 153 being 90% or less the arc length between the native commissures.

Alternatively, the curvature and arc length of the annulus section 122 in a transverse plane may span an arc length that mimics the posterior mitral annulus, which is approximately ⅗ the circumference of the mitral annulus 62, or may span the portion of the mitral valve annulus connected to the native posterior mitral valve leaflet 64 including the posterior scallops P1, P2, P3 and optionally the commissural scallops 55, 56. In some embodiments the annulus section 122 may be sized slightly larger than the native posterior annulus to apply a radially outward force against the annulus 62, which may facilitate stability and anchoring or containment of the native posterior leaflet 64. In some embodiments the annulus section 122 may be sized to be the same or slightly less than the native posterior annulus so minimal or no force is applied to the native annulus and yet the native posterior leaflet is contained between the prosthetic valve and native tissue.

A curvature 155 of the annulus section 122 in a sagittal plane, as shown in FIG. 4B, may include a concave curvature on the posterior side 104 of the frame 120 created by shape setting annulus section 122 to closely resemble curvature of the native posterior annulus so the curved annulus section mates well with the native annulus. Furthermore, the curvature of the structural frame 120 in a sagittal plane may include a flaring out of the atrial section and ventricle section. This curvature helps to secure the prosthetic valve in place, for example along with the apposition force between the annulus section and native annulus that is applied by the arms anchored in the anchoring range of the heart (e.g., may include trigones, or region that spans from commissures to trigonal area). The curvature of the annulus section can help to ensure a maximum contact surface, so pressure is well distributed. The curvature of the annulus section may have a radius of curvature in a range of 3 mm to 10 mm. The atrial section 121 may extend from the annulus section 122 in a posterior direction to conform to the surface of the atrium, which may further secure the prosthetic valve and spread contact force across the surface. A posterior view from the external side of the frame of FIG. 3B is shown in FIG. 4D.

The atrial petals 130 may curve outwards from the central axis 106 (e.g., to an angle 109 in a range of 30 to 60 degrees with respect to the central axis 106). Curvature of the atrial petals 130 in a vertical plane may include a bending of the atrial petal apexes 133 toward the central axis 106. For example, a superior aspect (e.g., the top 1 to 8 mm) of the atrial petals may be bent or curved inward (e.g., toward the central axis 106 at an angle to the lower aspect of the atrial petals at an angle in a range of 30 to 60 degrees (e.g., 40 to 50 degrees). The curvature of the atrial petals may closely resemble the curvature of the native atrium, which may help to evenly spread pressure and reduce risk of traumatic forces applied to the atrium, which further may improve safety and improve ability for tissue ingrowth to secure the prosthetic. This curvature may also facilitate delivery of the prosthetic valve which may be held by a delivery tool via the delivery interface holes 138.

The ventricle cell vertical section 123 may flare away from the central axis 106 in the vertical plane (e.g., at a ventricle section angle 111 in a range of 5-30 degrees). This shape may be adapted to hold and position a prosthetic posterior leaflet 64 and optionally commissural leaflets 55, 56 inferior to the annulus section 122 in the left ventricle 58 and wider than the annulus section, which may optimize geometric orifice area. For example, when the prosthetic posterior leaflet is in an open position it may be moved radially away from the mitral orifice, so the geometric orifice area of the prosthetic is equal to or very close to the geometric orifice area of a native non-diseased mitral valve. Concurrently, the ventricle section 123 may be flared outward from the annulus section 122 to not apply significant unnatural forces to the chordae, which may preserve as much as the native heart function as possible, nor to impede blood flow on the exterior side 108 of the prosthetic valve within the left ventricle, which may allow sufficient rinse of the area in the left ventricle external to the prosthetic valve. The prosthetic valve may be adapted so that the ventricle section is flared to provide maximum GOA and yet remain a minimum distance from the posterior ventricle wall in a range of 3 to 20 mm (e.g., 4 to 15 mm, 5 to 10 mm) during ventricular diastole to define a space and allow sufficient rinsing of the space. This flared configuration of the ventricle section may allow the prosthetic leaflets to be held in an appropriate position without impeding blood flow entering the left ventricle or leaving it.

Attachment features of the arms 210 may include arm suture attachments 139 in the atrial section 121, wherein a wire (e.g., upper wire) forming a horizontal arm is bent in an upward vertical direction and sutured to the arm suture attachments 139. Arms may further be connected to the structural frame in the ventricle section 123, wherein a wire (e.g., lower wire) forming the horizontal arm is bent in a downward vertical direction and sutured or connected in other ways such as crimped, welded, locked into a mating feature, force fit, or friction fit, to a part of the ventricle section 123 of the structural frame 120, such as an annulus cell strut a ventricle cell vertical strut 163, 164, or leg suture attachments 171.

Attachment features for anchoring the leg 190 to the structural frame 120 are shown in FIG. 4A, FIG. 8A and FIG. 8B, wherein the attachment features 171 may include adaptations to the frame 120 that allow connection of an anchoring leg (e.g., a leg that is wire-formed or laser cut) to the frame in a secure manner that prevents displacement or rotation of the leg relative to the frame. Leg attachment features may be part of a laser cut strut of the frame such as an annulus strut or ventricle strut. Leg attachment features may include suture attachment points, holes through which ends of the leg are passed 171A, 171B, indents for containing sutures 171, holes through which sutures are threaded, or other features that are friction fit, form fit, press fit, for example. Leg attachment features may be distanced horizontally and vertically from one another to securely connect the base of the leg to the frame.

### Leg(s)

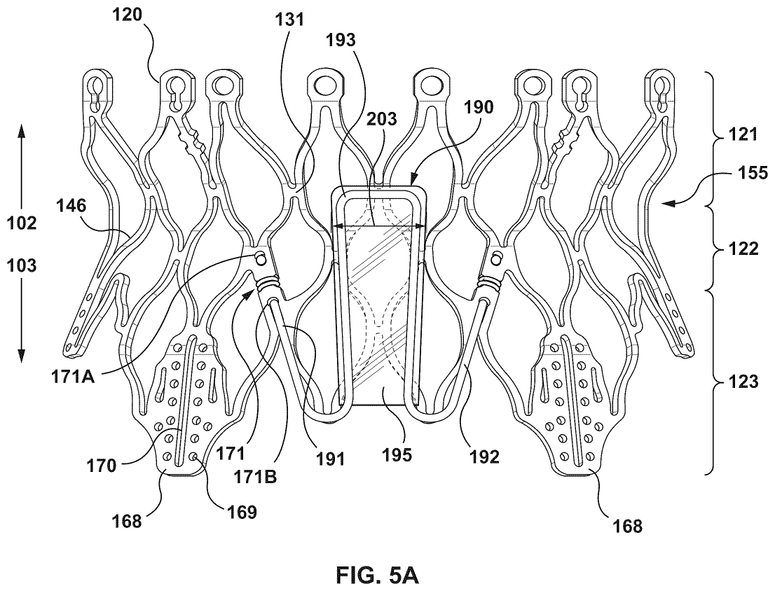

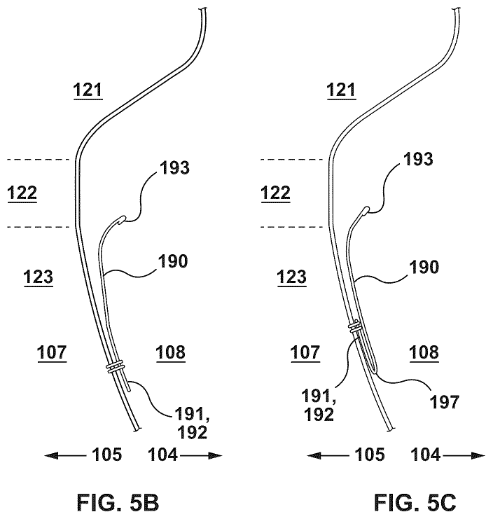

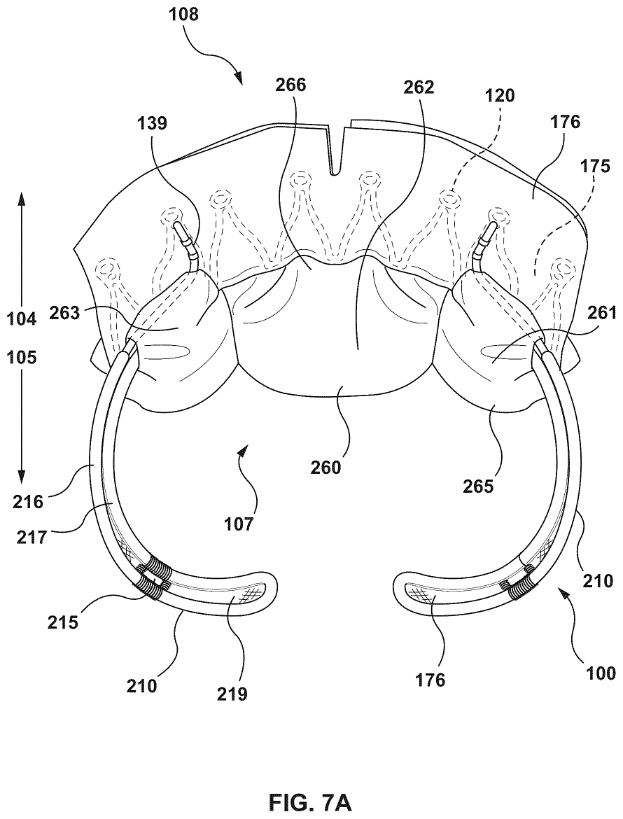

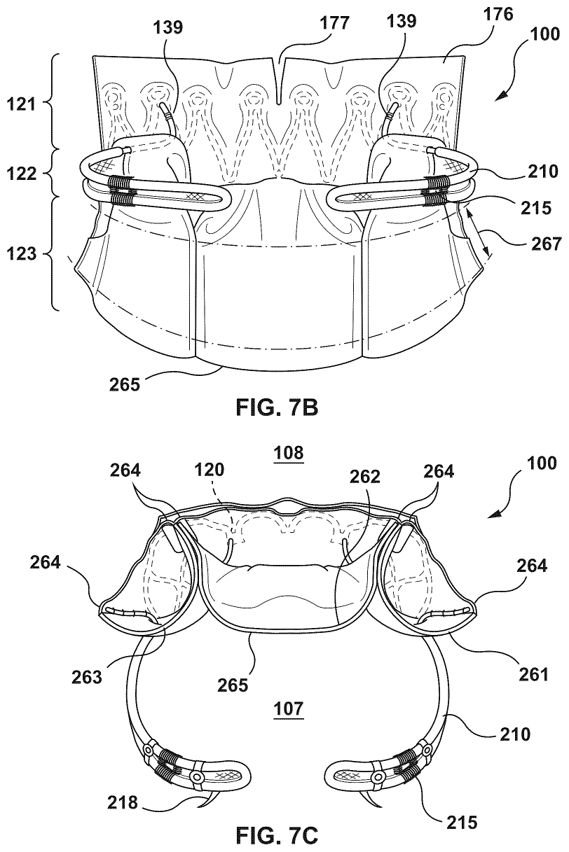

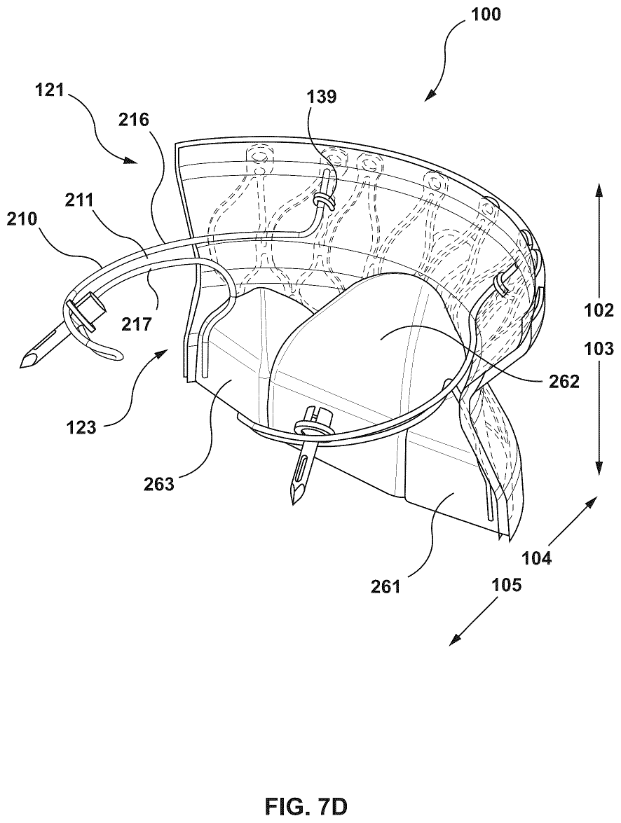

The prosthetic valve may have at least one anchoring leg 190, which may be connected to the structural frame 120, for example to a ventricle section 123 or annulus section 122 of the structural frame 120, and extend from the external side 108 of the structural frame (e.g., posterior 104 to the frame 120) at least in part in a superior direction, as shown in FIG. 5A and FIG. 7B. The anchoring leg may function to clip the prosthetic valve over native tissue, which may include compressing or moving at least a portion of the native posterior leaflet 64 out of the mitral orifice, or straddling the chordae bundle (for example passing between native chordae); anchor the prosthetic valve preventing it from being dislodged under the pressures of a beating heart, in particular ventricular pressure; self-center the prosthetic valve so it seats itself correctly in the native tissue by passing between the chordae tendineae 65 that are connected to the lateral papillary muscle 66 and the chordae tendineae 65 connected to the medial papillary muscle 67, particularly with just one leg; and stabilize the prosthetic valve rotationally.

By capturing and condensing at least a portion of the native posterior leaflet between the prosthetic valve and the native posterior mitral annulus, the tissue contributes to creating a fluid seal preventing perivalvular leaks.

The native posterior leaflet, or at least a portion of it may be moved posteriorly by the prosthetic valve and held in a fairly natural open and elongated state, which may prevent tension in the native chordae that are connected to the posterior leaflet and thus prevent applying unnatural forces to the papillary muscles 67, 67. This may prevent unnatural forces applied to chordae connecting the papillary muscles to the native anterior leaflet 63 so it can function properly and fully coapt against the prosthetic posterior leaflets.

The anchoring leg 190 may be made of a wire (e.g., superelastic Nitinol) as shown in FIG. 5A, or be part of the laser-cut Nitinol structural frame 120.

The anchoring leg 190 may include a first end 191 and a second end 192, each connected to the structural frame 120, and connected therebetween by a middle section that extends in a superior direction with a curved bend 193 at the superior apex (FIG. 5A). The first and second ends 191, 192 may be bent upward in a superior direction and angled away from the center of the leg, which may give the leg 190 a secure connection to the frame and may also affect the behavior of the leg as it transitions from a compacted delivery state to a deployed state. The most inferior 103 aspect of the leg 190 may be positioned at or near the same height of the inferior aspect of the structural frame 120 that the leg adjoins to. The most inferior 103 aspect of the leg 190 may be positioned more superior 102 to the P2 leaflet connection frames 168. The leg 190 may have a height 204 in a range of 10 mm to 20 mm. The apex 193 of the leg 190 may be positioned at the height of the superior annulus struts 131 or within 2 mm of their height. The leg width 203 may be adapted to pass between the medial and lateral chordae tendinea 65 (e.g., in a range of 2 to 8 mm, in a range of 3 to 5 mm). The middle section of the leg may have a radially outward bend 196, (e.g., having bend angle 196A in a range of 90 to 135 degrees, and a radius of curvature in a range of 1 to 8 mm), which may be adapted to seat into the AV groove 68 behind the native posterior leaflet and inferior to the mitral annulus 62, facilitate delivery by providing a wide space to capture and funnel the native posterior leaflet between the leg 190 and the ventricle section 123 of the frame 120, to spread contact forces, or to provide an eyelet loop for passing a tissue anchor through. The end of the leg may include a loop in the wire forming the leg. The loop may be adapted, for example have a curvature, that closely resembles the tissue surface in the AV groove where the leg contacts to disperse contact forces over more space, which may hold the prosthetic securely and reduce pressure applied to tissue.

A fabric sock 195 (e.g., made from felt, woven fibers, or stretchable material) may be connected to the leg over the Nitinol wire leg or laser-cut leg to further spread forces and reduce pressure to minimize traumatic forces on AV groove tissue, to encourage tissue ingrowth for long-term durability, and to reduce risk of ventricular or atrial puncture or undesired iatrogenic injury.

The apex 193 of the leg 190 may be tapered, as shown in FIG. 7C, which may facilitate a step of deploying the prosthetic valve and guiding the leg 190 between the lateral and medial chordae tendinea 65.

The leg 190 may have a radiopaque marker 200 which may be positioned on its apex 193, or the leg may be entirely radiopaque.

The leg 190 may have an additional inferior curve 197. FIG. 5B shows a leg 190 without an inferior bend wherein the middle section extends in a superior direction from the first and second ends 191, 192. FIG. 5C shows a leg 190 with an inferior bend 197, wherein the middle section extends in an inferior direction from the first and second ends 191, 192, then bends 197 before extending in a superior direction. This embodiment may provide greater height between the leg 190 and ventricle section 123 of the frame 120 for capturing native tissue.

The rounded apex 193 of the leg, optionally spanned by fabric, may provide a leg anchoring surface through which a tissue anchor may be passed through. This tissue anchor may also pass through an anchoring cell of the atrial section and through native tissue such as the annulus or posterior leaflet before passing through the leg anchoring surface.

In some embodiments, a prosthetic valve 100 may have more than one leg 190, or may have one center leg 190 and additional legs having different features. One single leg 190 may be advantageous because as it is deployed from a delivery sheath it may be easier to direct a single protracting leg through a space between chordae bundles without tangling the chordae; it may consume less space in a delivery sheath making it easier to advance through the sheath or allowing a smaller sheath; and it may allow the prosthetic valve 100 to self-center more easily. However, additional legs may provide greater anchoring or stability.

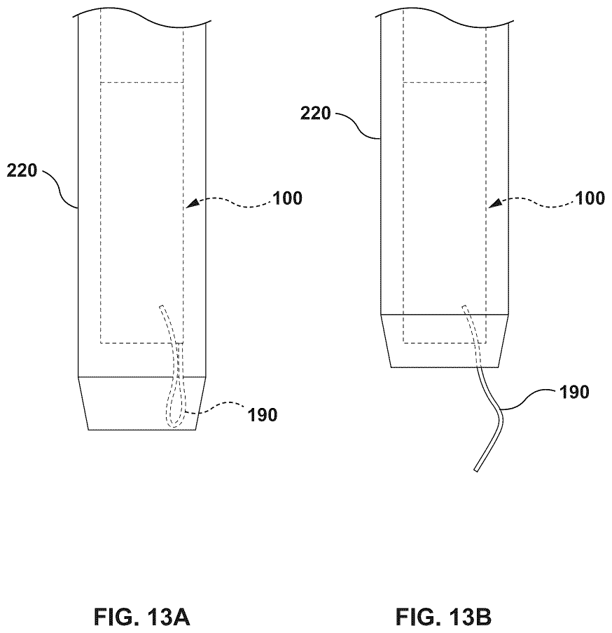

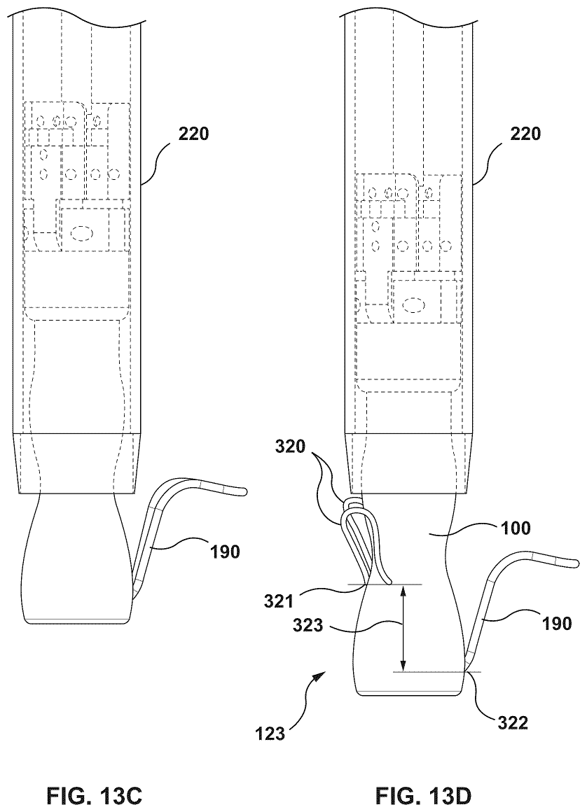

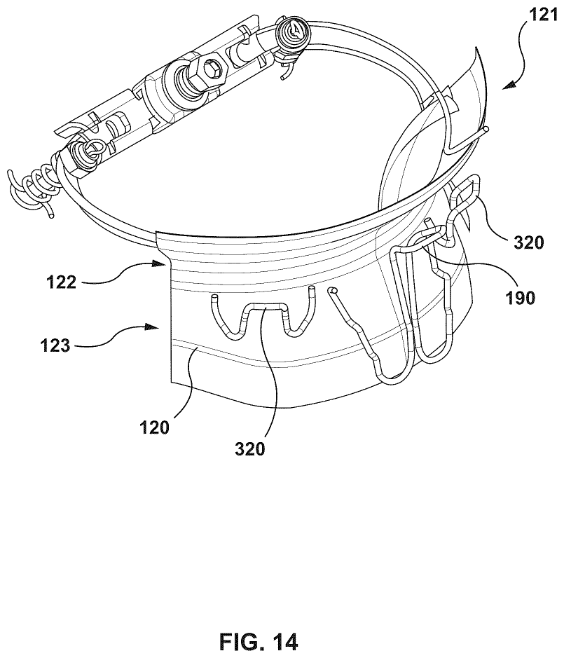

In some embodiments, a prosthetic valve 100 may have a primary leg 190 centered on the ventricle section 123 of the structural frame 120 that is configured to be positioned between chordae bundles, and additional legs, for example two additional legs 320 (FIG. 14). The additional legs 320 may protrude from the posterior side of the structural frame in an upward angle and have a length in a range of 2 to 6 mm (e.g., about 4 mm). They may be made from wire and attached to the structural frame, or alternatively be a part of a laser cut structural frame and shape set to protrude from the frame. They may be made from superelastic Nitinol. They may be covered in fabric such as felt to encourage tissue ingrowth and spread contact force over a larger area of the native annulus. A function of the additional legs 320 may be to improve stability of the prosthetic valve, hold it securely to the mitral annulus. Optionally, the additional legs may be made from a bioabsorbable material that temporarily provides support until tissue grows into other parts of the prosthetic valve. Optionally, the prosthetic valve may have a perivalvular curtain, for example in the form of a flexible sheet of biocompatible material (e.g. polyurethane, felt) connected to the prosthetic external to the legs 190 and 320, which functions to further reduce perivalvular blood leakage. The additional legs 320 may further function to hold the perivalvular curtain in place (e.g., in apposition with the annulus). The additional legs may have a different configuration than the primary leg 190 so that they deploy from a delivery sheath 220 after the primary leg 190 is deployed. In other words, the primary leg may be deployed when the delivery sheath 220 is retracted to the first position and the additional legs (e.g., 2) 320 may deploy when the delivery sheath 220 is retracted to a second position that is more proximal than the first position. For example, as shown in FIG. 14 the two additional legs 320 may be positioned at or near each side of the structural frame 120 on the external side of the ventricle section 123 or annulus section 122. Referring to FIG. 13D, a schematic illustration of a prosthetic valve 100 partially advanced from a delivery sheath 220, each additional leg 320 may extend from the external side of the ventricular section 123 at a second level 321 that is more superior to the first level 322 where the primary leg 190 extends from the ventricular section. The difference 323 between the first level 322 and the second level 321 may be in a range of 1 mm to 10 mm (e.g. 3 mm to 8mm). In some embodiments the primary leg may extend from the frame at the inferior end of the ventricular section and the additional legs may extend from the frame at a distance (e.g., difference 323) from the inferior end of the ventricular section. In some embodiments the shape of the ventricular section may be such that the inferior edge is lower in the middle of the posterior where the primary leg is connected and higher on the sides where the additional legs are connected and optionally the primary leg and additional legs may extend from the inferior edge of the ventricular section.

### Leaflets

A flexible prosthetic leaflet may be connected to the structural frame, for example to the ventricle section 123 and may mimic in part the function and geometry of the native posterior leaflet 64, primarily to seal against the native anterior leaflet 63 when the left ventricle is full or contracting to prevent or reduce regurgitation of blood back into the left atrium. Functions of the prosthetic leaflet may include: Providing a seal against the anterior leaflet 63 during ventricular systole; Opening fully to maximize GOA during ventricular diastole (in particular, during ventricular filling and atrial contraction); Providing stability of the prosthetic valve when in a closed position by directing the force applied by ventricular blood pressure evenly around the posterior mitral annulus. Together with the leg 190 and arms 210, this stabilizing force may help to prevent the prosthetic valve from being pushed superiorly into the atrium, and from moving in a roll, yaw or pitch motion.

A prosthetic posterior leaflet may be made from a synthetic material (e.g., polyurethane) or a natural material such as porcine or bovine pericardium. The material may have physical properties similar to a native posterior leaflet including flexibility, strength, or the durability to withstand latest FDA standards for cycles of opening and closing, and hold a ventricular pressure. The prosthetic leaflet 260 may be made from one single piece of material shaped into one or more (e.g. 2, 3) scallops, which may be done by sewing pleats in the material to separate the scallops. The pleats between scallops may be free-floating, i.e. not connected to the structural frame, which may function to coapt with the native anterior leaflet more reliably, mimic the native posterior leaflet more accurately, fill with blood more evenly across the whole posterior leaflet, open and close with less force, and allow the prosthetic valve to collapse to a delivery configuration more easily. In this example, the single-piece posterior leaflet may have two flaps 264, one on each side, for connecting to the structural frame at the leaflet connection side frames 168A. The superior edge 266 may be connected to the structural frame and the inferior edge 265 may be free-floating allowing blood to enter the space created by the leaflet joined to the structural frame. Alternatively, the prosthetic leaflet 260 may be made from multiple pieces of material, for example, as three separate scallops as shown in FIGS. 6A and 6B.

The prosthetic posterior leaflet 260 may include three scallops that at least partially replace the three native scallops of the posterior mitral leaflet 64. These may include a P1 or lateral scallop 261, a P2 or central scallop 262, and a P3 or medial scallop 263.

FIG. 6A shows a two-dimensional pattern of a P1 and P3 scallop 261, 263, which may be identical in dimensions, and a P2 scallop 262. The two-dimensional leaflet patterns may have darts that are gathered and sewn to create a three-dimensional half-dome shape that may facilitate a billowing effect to capture blood during ventricular systole. For example, the P2 prosthetic scallop 262 may have two darts 270, and the P1 and P3 261, 263 prosthetic scallops may have one dart 271 each.

FIG. 6B shows a two-dimensional pattern of another design of a P1 and P3 scallop 261, 263, which may be identical in dimensions, and a P2 scallop 262.

One dimensional difference of the prosthetic posterior leaflets compared to a native posterior leaflet is that the coaptation zone 267 may be larger than a native leaflet's coaptation zone, which may function to improve the seal with the anterior mitral valve, in particular with patients who have functional MR wherein the leaflets are pulled apart from one another creating a leak. The extra coaptation zone length may also accommodate a range of valve dimensions that vary from patient to patient. For example, the coaptation zone 267 may be in a range of 4 mm to 10 mm (e.g., 4 to 8 mm, 5 to 8 mm) (see FIG. 7B and FIG. 8B). The coaptation zone 267 may be chosen to optimize a seal of a diseased mitral valve yet not be too large, which may negatively reduce GOA or require an increased valve gradient or opening force. The P2 scallop 262 may have a width 268 that is wider than the width 269 of the P1 and P2 scallops 261, 263. For example, the P2 scallop may have a width in a range of 1.3 to 1.8 times the width of the P1 and P3 scallops. Each leaflet may have two side flaps 264, an inferior edge 265, and a superior arc 266.

FIG. 7A shows, from a superior perspective, a prosthetic posterior leaflet 260 with scallops 261, 262, 263 attached to the structural frame 120. The structural frame 120 may be clad with a membrane layer 175 (e.g., polyurethane) and may be further clad with a fabric layer 176 at least on the external side 108 of the structural frame 120. The superior arc 266 of each scallop may be sewn to members of the structural frame, for example to the superior annulus cell struts 131, the atrial petal struts 132, or annulus cell struts 146, or to the membrane 175 or fabric 176 layers.

FIG. 7C shows from an inferior perspective of the prosthetic posterior leaflet scallops 261, 262, 263 attached to the structural frame 120. The side flaps 264 of each scallop may be passed through a leaflet slot 170 (FIG. 4A) or around a side of a leaflet connection frame 168 from the internal side 107 to the external side 108. Sutures pass through the side flaps from the external side 108 and through leaflet eyelets 169. This provides a robust connection of the side flaps 264 to the frame in a location of the scallops 261, 262, 263 that experiences high levels of stress when in use; since the side flaps 264 of the P2 scallop 262 and the adjacent scallops are passed through the leaflet slots 170 the edges of adjacent prosthetic scallops are pressed together, which may provide a durable seal and prevent blood leakage. A large amount of the force is applied evenly across the side of the leaflet slot 170 instead of on the sutures. The inferior edge 265 of each scallop is longer than the straight distance between the leaflet connection frames that the scallop is connected to causing the inferior edge 265 to billow away from the structural frame 120 in a deployed configuration creating a cavity in the scallop that is only open at the inferior edge 265, which allows the scallops to fill with blood when pressure in the left ventricle is greater than in the left atrium. The scallops are flexible enough to be deflated and pressed against the structural frame, pushing out any blood held in the scallop cavities, when atrial pressure exceeds ventricular pressure. Since the ventricular section 123 is flared radially the scallops are moved out of the path of blood through the mitral orifice when deflated to maximize GOA.

The design of the leaflets in combination with the structural frame and other features as disclosed herein, which may allow a maximum diastolic GOA may allow the prosthetic valve to establish a diastolic pressure gradient between the left atrium and left ventricle that is less than or equal to 5 mmHg (e.g., no more than 4 mmHg, no more than 3 mmHg, no more than 2 mmHg, no more than 1 mmHg, 0 mmHg). The diastolic GOA with the prosthetic valve implanted may be at least 90% (e.g., at least 95%, 100%) of the diastolic GOA of the native heart without the prosthetic valve implanted. In some patients having a native heart with a constricted GOA as well as mitral regurgitation the prosthetic valve may be sized and used to enlarge the mitral annulus and increase GOA, for example by an amount in a range of 0% to 10%. In some patients having mitral regurgitation due to an enlarged mitral annulus, the prosthetic valve may reduce the GOA while providing a diastolic pressure gradient between the left atrium and left ventricle that is less than or equal to 5 mmHg (e.g., no more than 4 mmHg, no more than 3 mmHg, no more than 2 mmHg, no more than 1 mmHg, 0 mmHg).

### Arms

A prosthetic valve 100 may have two arms 210 extending laterally from the sides of the atrial section 121 of the structural frame to anchor the prosthetic valve to tissue or provide stability. As shown in FIG. 8A, the arms 210 extend from the sides of the atrial section 121 and/or annulus section 122 of the structural frame 120 and have a curvature in the transverse plane that approximately follows the curvature of the native mitral annulus within a few millimeters superior to the mitral annulus and reaches the trigones 52, 53, which contain strong, fibrous tissue. The arms 210 may have anchor points 215 which may be anchored to tissue such as the trigones. Anchor points 215 may be spaces such as eyelets (FIG. 7A) or slots (FIG. 8A) through which a tissue anchor 280 may be passed and further passed into the tissue. As shown in FIG. 8A anchor points 215 may be slots 211 that provide a range of locations through which a tissue anchor 280 may be passed, which advantageously allows tissue anchors to be placed in a trigone for a range of native tissue geometries that varies from patient to patient or from lateral to medial sides. In some embodiments, the prosthetic valve 100 may be allowed to sit in the desired implant position and be anchored to trigones while applying minimal to no tension or compression of native tissue, for example to the mitral annulus 62, to the annulus between the trigones 52, 53 and the posterior annulus, to the anterior annulus, to the aortic valve, to the bundle of His, to blood vessels of the heart, to the anterior leaflet, or to other tissue structures, which may facilitate a goal of minimal physical modification of native structures. In this configuration the tissue anchors 280 placed through arm anchor points 215 may function to prevent the prosthetic valve 100 from migrating or from moving in pitch, roll or yaw directions. The arms 210 (see FIG. 7D) may be constructed from a wire such as superelastic Nitinol forming a loop wherein each end of the wire is connected to the structural frame 120, such as the arm attachment features 139. The wire loop may form a slot 211 with a substantially consistent width (e.g., in a range of 0.5 to 3 mm) for a length in a range of 5 to 35 mm, which may be configured to align with trigones 52, 53 in a variation of locations and for passage of a shaft of a tissue anchor 280 and engagement of a tissue anchor flange 281. The arm 210 may have at least one stabilization bend 212 for securely connecting the wire to the structural frame 120. The arms 210 may be joined to the structural frame via a form fit, force fit, friction fit or the like into a mating receptacle of the structural frame and optionally may be further connected with adhesive, welding, sutures, crimping or the like. Alternatively, the arms may be fabricated from the same component as the structural frame and be integrated with the structural frame as a single component.

Alternatively, the prosthetic valve 100 may have at least one anchor point in each of two arms for anchoring to trigones, wherein the arms (e.g., made of superelastic Nitinol) transmit force on the structural frame in a posterior direction (e.g., away from the trigone anchors) so the annulus section 122 maintains stable contact and/or applies pressure to the posterior mitral annulus 62. This may cause a little tension in a portion of the mitral annulus, particularly the posterior portion between the trigones, which may compress at least portions of the native posterior leaflet. however, minimal force may be applied to native structures such as the aortic valve, bundle of His, blood vessels of the heart, anterior leaflet, papillary muscles, and other structures of the heart. To accomplish this, the tissue anchors 280 may be held in place in the arm anchor points 215 so they do not slide within the slot 211, for example with eyelets (FIG. 7A), with a bend in the arm adjacent to the anchor points, or with a fabric connected to the arm through which an anchor can be punctured. The arms 210 may be securely connected to the structural frame 120 so forces are transmitted to the atrial section 121 and ventricle section 123 of the frame 120. For example, as shown in FIG. 7D, arms made from Nitinol wire may be connected to the frame at multiple points to prevent pivoting or dislodgement. The arm 210 may have an upper wire 216 that is bent in a superior direction and is connected (for example sutured, welded, crimped, inserted through holes, friction fit) to the structural frame at arm attachment points 139 in the atrial section 121; and the arm may have a lower wire 217 that is bent in an inferior direction and is connected to the structural frame at arm connection points in the annulus section 122 and the ventricle section 123. The posterior force may be created in part by resilient deformation of the superelastic Nitinol arms, selecting an appropriate size of prosthetic valve to fit the patient's heart, placing tissue anchors in selectable arm anchor points.

The arms 210 along with the rest of the prosthetic valve 100 may form a structure to which another subsequent valve may be implanted inside the prosthetic valve 100 in the case of further progression of MR disease after the prosthetic valve 100 is implanted. The arms, frame, and trigone tissue anchors may provide a stable, semi-rigid orifice into which another prosthetic valve can be implanted to prevent embolization.

Arms 210 may be fabricated from the same wire that a leg 190 is fabricated from.

Arms 210 may be fabricated from the same material as the structural frame such as a laser cut Nitinol sheet.

Arms 210 may be covered in a fabric (e.g., felt) to enhance tissue ingrowth, reduce contact pressure or facilitate connection to a tissue anchor.

A prosthetic valve 100 may be provided with tissue anchors 280 held in anchor points 215 of the arms 210, for example slidably held in slots 211 or eyelets 213.

Arms 210 may have multiple eyelets providing a variety of anchoring points 215 (FIG. 7C).

Arms 210 may each have barbs or teeth 218 (FIG. 7C) protruding from anchor points or distal ends of each arm. The barbs or teeth may be angled distally so that a compressive force pushing the frame posteriorly on to the native posterior annulus pushes the anchor points of the arms anteriorly which helps to engage the barbs into tissue, such as the trigones or annulus.

Arm length may be sized to extend only to trigones or slightly beyond, so they don't apply any force to the aortic wall, yet apply back force to the structural frame against the posterior annulus. Since the function of the arms of pressing the frame into the posterior annulus is important, dimensions of the arms may be considered with respect to the frame and native valve dimensions. For example, the arms 210 may each have a length in a range of 15 to 50 mm (e.g., 15 to 30 mm, 20 to 40 mm, 30 to 45 mm). A method of selecting a prosthetic valve to be implanted in a patient from a group of valves having varying sizes may comprise selecting a prosthetic valve having a major arc length between each arm anchor point 215 that is within an amount in a range of 0 to 10 mm of a major arc length between the center of the patient's left trigone and the center of the patient's right trigone.

Anchoring features for connecting to frame may include upper and lower bends and vertically and horizontally spaced suture locations, to securely hold the arms and prevent rotation, pivoting or sliding. For example, the arm's upper bend may be secured to the atrial region of the frame and the lower bend may be secured to the ventricle region of the frame, which may allow the forces applied by the arms on to the frame to be applied to the atrial and ventricle regions.

The arms extend in a substantially horizontal plane that aligns with mitral annulus and holds the frame stable while slight compressive force in arms apply pressure between frame and native posterior annulus and leaflet (e.g., at least a portion of P1 and P3, plus all of P2) to contain the posterior leaflet. The arms are flexible to move with the annulus as it changes from systole to diastole and yet maintain apposition between the back of the frame and the posterior annulus.

The vertical arm angle, i.e., the angle of a line between the upper 216 and lower 217 wires of each arm with respect to a vertical central axis, may be smaller (e.g., 0 to 30 degrees, 0 to 20 degrees, 0 to 10 degrees) in the arm portion and larger (e.g., 20 to 70, 30 to 60, 40 to 50 degrees) in the region containing anchor points 215. The smaller angle in the arm portion may preferentially transfer arm compression into a horizontal projection instead of vertical projection to better secure the prosthetic valve and prevent migration. The larger angle in the region containing anchor points 215 facilitates delivery of anchors into the anchor points and aligns the anchors 280 to enter the target anchoring tissue (e.g., trigones) and avoid entry through thin, fragile structures and to avoid puncturing the aorta or atrial wall. The larger angle in the region containing anchor points 215 may also allow the anchors to be inserted at an angle (e.g., perpendicular +/−10 degrees to said larger angle), which may allow the arms to transmit a force vector on the anchors that is not parallel to the axis of the anchors, which may improve ability of the anchors to remain secure.

The arms may be covered with fabric (e.g., felt), which may function to encourage tissue ingrowth and to prevent the anchor from sliding in the channel.

The tips 219 of arms may be bent inward to minimize trauma or forces applied to aortic curtain 72.

The tips 219 of the arms may be linked together forming a loop that is connected to the structural frame and approximately follows the circumference of the native mitral annulus. For example, as shown in FIG. 8D, the arm tips 219 may be joined by an anterior arm bridge 209, which may be flexible with little or no elastic resiliency, for example made from fabric or a string. Alternatively, the anterior arm bridge 209 may be made from the same material or be the same component as the arms 210, such as Nitinol wire or laser cut Nitinol and have a preformed shape that avoids applying pressure to the aortic curtain. The anterior arm bridge 209 may function to prevent the arms 210 from splaying apart, which may prevent distortion of the mitral annulus that can lead to progression of mitral regurgitation. The anterior arm bridge 209 may function, along with the arms and structural frame, to provide an annulus framework into which a different prosthetic valve may be implanted if additional intervention is required.

Optionally, other features to prevent anchor sliding may be included in the arms, such as: sutures tied around arm wires, twist in arms so wires cross preventing sliding of anchor, protrusions or indents to prevent sliding or to hold sutures, arm may be laser cut Nitinol with holes for anchors. Optionally arms may be integrated into laser cut frame as one piece.

### Anchors

Tissue anchors 280 may be used to provide prosthetic valve stability or prevent migration, wherein the anchors 280 may be passed into or through native tissue to connect an anchor point of the prosthetic valve to the tissue.

A target native tissue anchor position may include one or more trigones 52, 53, which may be anchored to anchor points 215 of the arms 210.

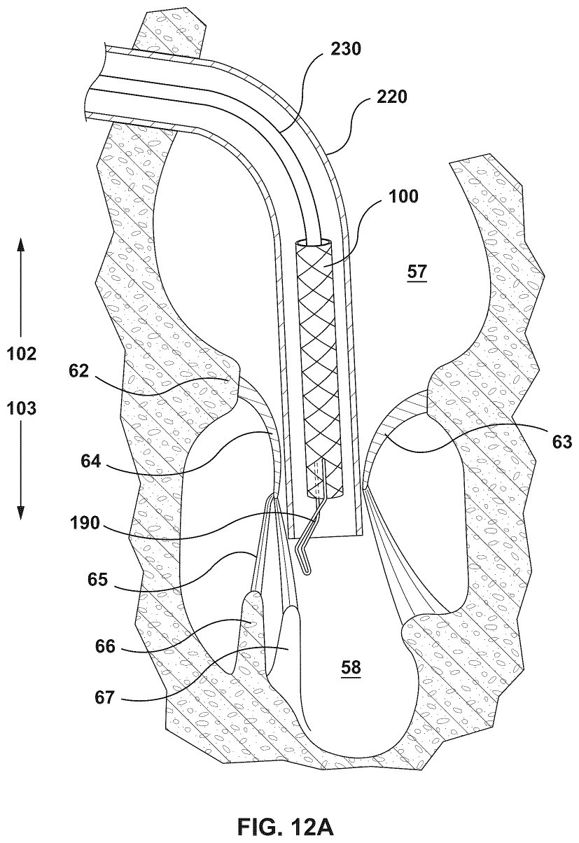

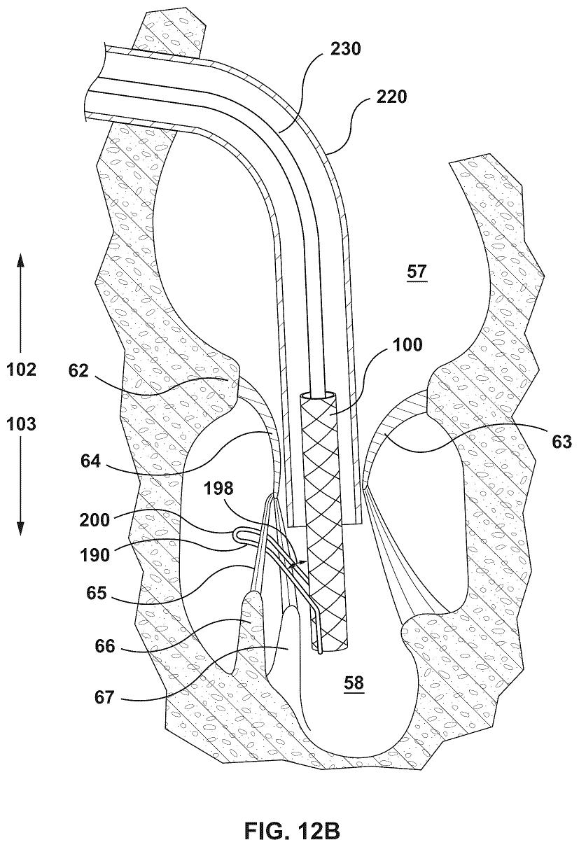

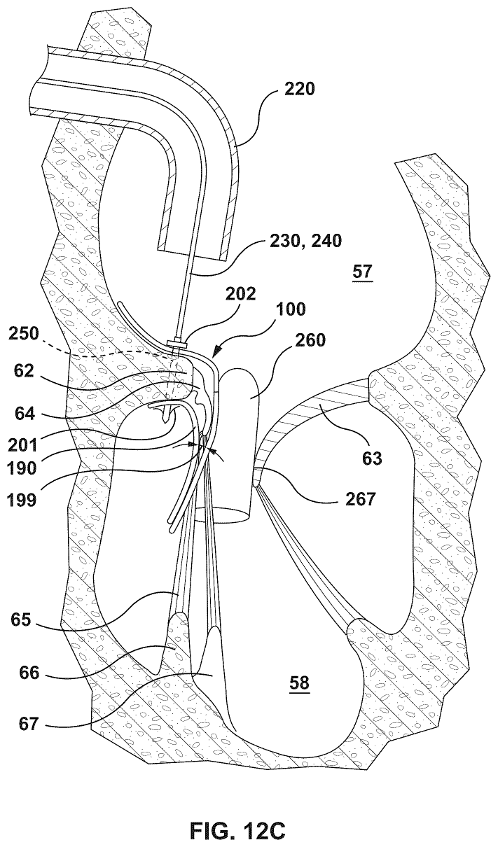

A target native tissue anchor position may include the posterior annulus or native posterior leaflet, wherein one or more anchors 280 may be anchored to one or more anchor points 201 on a leg 190 and one or more anchor points 202 on atrial petals 130 (FIG. 12C).

A prosthetic mitral valve may have anchor points only in two arms which align with or near the native trigones.

Complete anchoring in this embodiment may include a combination of features including: a tissue anchor placed through an anchor point in each of the two arms and into trigones with the arms sized and configured to apply a force that pushes the annulus section (e.g., C-shaped) of the structural frame into engagement with the posterior annulus, and to resist tilting motion or pivoting of the frame about the annulus; a leg such as a single leg centered on the posterior of the structural frame and configured to clamp on to the native P2 leaflet and engage with the AV groove to prevent lifting of the prosthesis (e.g., migration into the left atrium) or pivoting about the arm anchors; a flared atrial section 121 and ventricle section 123 with respect to the annulus section 122 to follow the contour of the heart providing evenly spread apposition and resisting motion; and a fabric covering 176 that promotes tissue ingrowth.

A prosthetic valve 100 may be provided with tissue anchors positioned in an anchor point and ready to be passed into or through tissue. For example, tissue anchors may have two flanges holding an anchor point between them.

Alternatively, tissue anchors may be provided separately from the prosthetic valve and advanced into an anchor point during implantation, for example after the prosthetic valve 100 is deployed from a delivery sheath and positioned in the target location at the posterior mitral valve.

Tissue anchors 280 may be deliverable with an endovascular anchor delivery catheter 240 and may include a tissue penetrating tip 282, an anchoring feature 283 and a flange 281 for grasping tissue and the prosthetic valve 100 between the anchoring feature and flange, and a delivery feature 284 for interfacing with a delivery catheter.

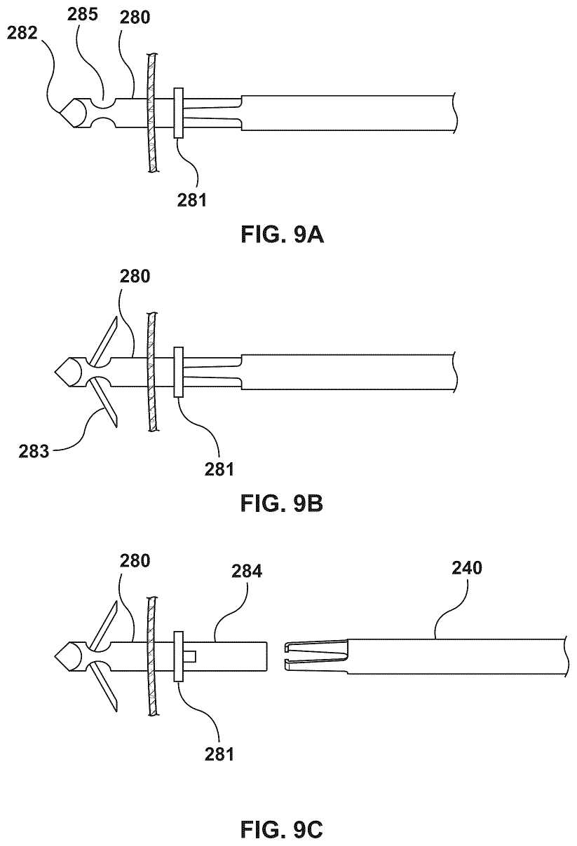

An example of a tissue anchor 280 as shown in FIG. 9A to 9C may have a generally tubular shape with a penetrating tip 282 in the form of a sharpened rod (e.g., sharpened with a trocar tip, beveled tip, pencil tip). The anchor may have an internal lumen which contains and anchoring feature 283 in the form of deployable barbs that are deployed by a delivery catheter 240, for example by pushing a rod in the delivery catheter that advances the deployable barbs 283 within the lumen pushing the barbs out of side holes 285 of the anchor. The barbs 283 may be made from a superelastic Nitinol wire that has sharp ends and may be bent so the ends are directed proximally or toward the flange 281. The wire may form a loop that is contained in the anchor's lumen, which prevents the deployed ends of the barb from rotating and provides a connection to a delivery feature 284 such as a thread or rod of a delivery catheter 240.

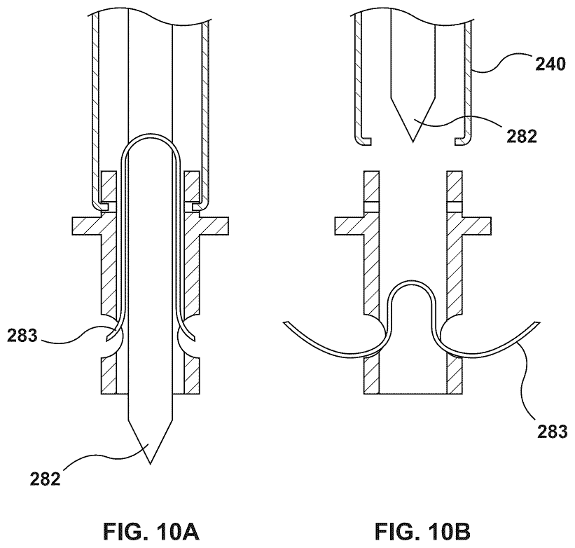

Alternatively, a penetrating tip 282 may be part of a delivery tool that is exposed by extending through a lumen of the anchor during implantation and removed after the anchor is set, as shown in FIGS. 10A and 10B.

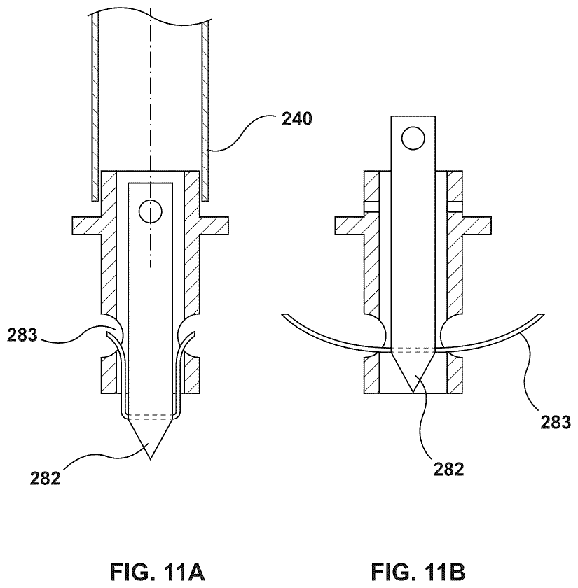

Alternatively, a penetrating tip 282 may be retractable into the tissue anchor, wherein the tip 282 is slidably contained in a lumen of the anchor and extends from the end of the anchor during delivery and is retracted into the lumen after delivery. The retractable tip may be connected to deployable barbs which are deployed from the sides of the anchor when the tip is retracted, as shown in FIGS. 11A and 11B.

An anchoring feature 283 may alternatively be in the form of a helical screw, non-deployable barbs, a spiraling wire, an expanding joint.

Tissue anchors may be adapted to be passed through or connected to anchor points on prosthetic valve arms 215, as shown for example in FIG. 7D.

An anchor delivery catheter 240 may be the same component as a prosthetic valve delivery shaft 230 contained withing a delivery sheath 220

Tissue anchors may be adapted to be passed through or connected to an anchor point on a leg 201 and atrial petal 202 and through tissue such as the posterior annulus 62 as shown for example in FIG. 12C.

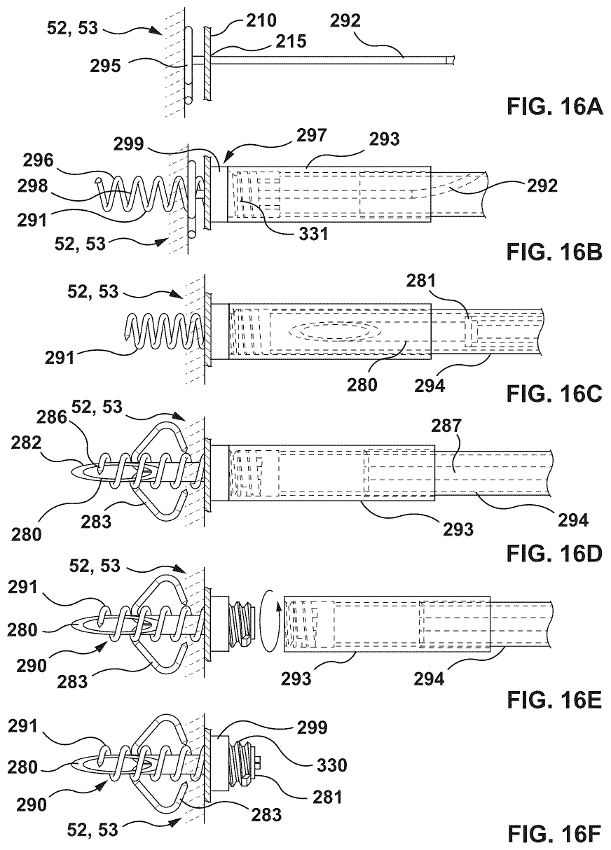

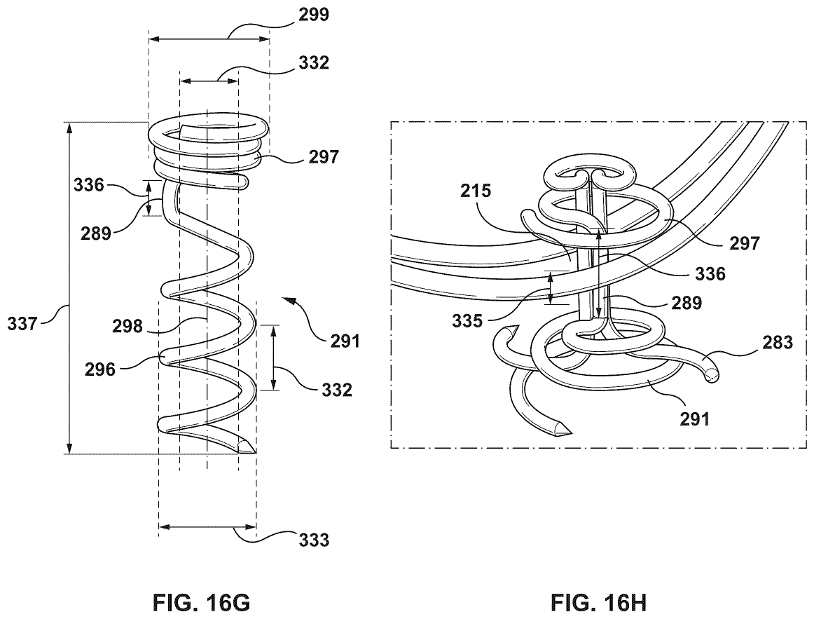

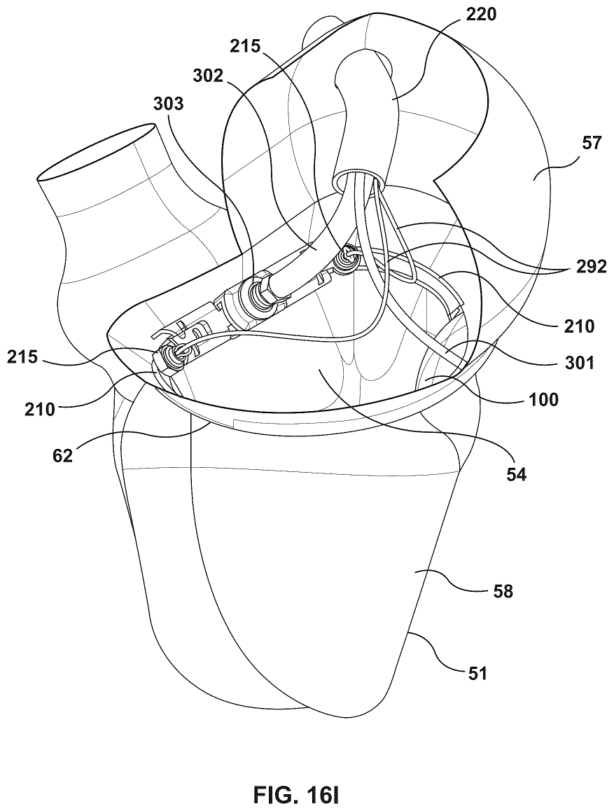

An alternative design of a tissue anchor system 290 shown fully implanted state in FIG. 16F, includes a corkscrew mount 291, a tissue anchor pillar 280, and a deployable barbs 283. This tissue anchor 290 may have features of the anchor 280 shown in FIGS. 9A to 11B such as a tubular penetrating tip pillar 280 with a flange 281 and a central lumen and side holes 285 that barbs 283 are deployed from. Additionally, the anchor 290 has a corkscrew anchor mount 291 through which the penetrating tip pillar 280 is inserted followed by deployment of barbs 283 from the pillar. The combination of the corkscrew 291 and penetrating tip pillar 280 with deployable barbs 283 may hold the valve to the tissue firmly and prevent the anchor from pulling out making a stronger anchor. The combination may also improve insertion of the anchor system. For example, the corkscrew anchor mount may have a thinner wire gauge, optionally sharpened at its end, than the penetrating tip pillar, and because its vector for penetrating tissue is not perpendicular to the tissue surface it may penetrate into tissue easier with less of a tenting effect. Once engaged with tissue, the corkscrew may pull the tissue toward the delivery catheter as the corkscrew is rotated instead of pushing the tissue away. Once the corkscrew 291 is inserted it may function to hold tissue steady and in apposition with the prosthetic valve (e.g., arm 210) while advancing the penetrating tip pillar 280 through the center of the corkscrew 291, in particular preventing the tissue from tenting. The penetrating tip pillar 280 may add strength to the anchor system 290 since it is made of a higher gauge material and is less flexible than the corkscrew alone. Furthermore, the barbs deployed from the penetrating tip may prevent the anchor from loosening by turning and decoupling from the tissue.

A sequence of deploying the anchor 290 through a part of a prosthetic valve (e.g. an arm 210, an anchor point, a part of the structural frame) into native tissue (e.g. a trigone 52, 53) is shown in FIGS. 16A to 16F as well as in a flow chart shown in FIG. 16J. The anchor system 290 may have features that allow it to be delivered through a transvascular anchor delivery catheter. The anchor delivery system may include an anchor delivery guidewire 292, and delivery catheters including an outer anchor catheter 293 and an inner anchor catheter 294.

The anchor delivery guidewire 292 may be a relatively small gauge guidewire (e.g., 0.018″) with a loop 295 formed on its distal end. The guidewire 292 is positioned through an anchor point of the prosthetic valve (e.g., an arm anchor point 215, a leg anchor point 201, an atrial petal anchor point 202, or an anchor point on another location of the valve) with the loop 295 distal to the anchor point to temporarily hold it in place and the remaining length of the guidewire 292 optionally contained in the valve delivery sheath 220, and optionally contained in an outer anchor catheter 293 that may be contained in the valve delivery sheath 220 during the anchor implant process. The loop 295 may be formed with a diameter that is larger than the diameter of the anchor point opening but compact enough to be easily manageable (e.g., the loop 295 diameter may be greater than but not more than twice the anchor point opening diameter). The loop 295 may be in a plane that is perpendicular to the longitudinal axis of the guidewire and/or parallel to the tissue surface. The loop may be in the form of a spiral in a plane with the longitudinal axis of the guidewire in or toward the center of the spiral. The loop 295 may be an incomplete loop (i.e. the distal end is free) so that when the guidewire is pulled proximally from the proximal end of the guidewire, the loop elastically deforms and is pulled out of the anchor point opening. The guidewire 292 may have a superelastic core (e.g., Nitinol).

The corkscrew anchor mount 291 is shown being implanted in FIGS. 16B to 16F and as an individual component in FIG. 16G. The corkscrew anchor mount 291 has a corkscrew-shaped wire 296 connected to a corkscrew head 297, optionally with a sharpened distal tip. A corkscrew lumen 298 passes through the head 297 and the corkscrew-shaped wire 296. The guidewire 292 slidably passes through the corkscrew lumen 298 allowing the corkscrew anchor mount 291 to be advanced over the guidewire 292 and through anchor point 215 on the prosthetic valve. For example, the corkscrew lumen 298 may have an inner diameter 332 of about 2.26 mm and the guidewire may have a diameter of about 0.45 mm. The corkscrew-shaped wire 296 may be made with a wire having a diameter in a range of 0.4 mm to 0.6 mm (e.g., about 0.46 mm). The helical shape of the corkscrew may have an outer diameter 333 (e.g., in a range of 2.5 to 3.5 mm, about 3 mm) that is larger than the opening of the anchor point (e.g., in a range of 1.5 mm to 3.5 mm, about 1.78 mm) or alternatively may be equal to or slightly smaller than the opening of the anchor point. The helical shape of the corkscrew may have a pitch 334 that is larger than the thickness 335 of the anchor point 215 (FIG. 16H) on the structural frame allowing the corkscrew anchor mount 291 to be advanced through the opening of the anchor point even when the corkscrew OD is larger than the ID of the opening, for example the pitch 334 may be in a range of 1.5 to 2.5 mm (e.g. about 2 mm). The head 297 may have an outer diameter 299 that has a larger diameter than the opening of the anchor point 215 to prevent the head from passing through the anchor point opening. The corkscrew head 297 may have an OD in a range of 3 to 4 mm (e.g. about 3.6 mm) and be made by coiling the wire 296 into the head 297 having a tight pitch, an OD 299, and a lumen ID 332. Alternatively, the head 297 may be made from a machined ring attached to the corkscrew wire 296 and may have a coupling 330 (e.g., male threaded coupling) for temporary coupling to an outer anchor catheter 293 via a mating coupling 331 (e.g., female threaded coupling). For example, coupling 331 and coupling 330 may be tightened by turning the outer anchor catheter clockwise, which may also advance the corkscrew-shaped wire 296 into the tissue, in other words the coupling 330 may have threading that is in the same helical direction as the corkscrew-shaped wire 296. Optionally, the corkscrew-shaped wire 296 may be formed into a tapered helix. In one example, a tapered helix a corkscrew anchor mount 291 is narrower at its proximal end as shown in FIG. 16H. Alternatively, a tapered helix of is narrower at its distal end. Optionally, the corkscrew anchor mount 291 may have a neck 289 as shown in FIG. 16G. The neck 289 is intended to traverse the anchor point 215 of the structural frame when the helical corkscrew portion is fully embedded in tissue on a distal side of the anchor point and the anchor head 297 is on the proximal side of the anchor point. The neck functions to provide a haptic clicking feeling transmitted through the outer anchor catheter 293 that indicates that the anchor is fully implanted, to allow the anchor to engage with the tissue and the anchor point securely while positioned with its central axis approximately transverse to the surface of the tissue and anchor point 215, and to reduce risk of the anchor disengaging from tissue by turning out. The neck 289 may be formed by bending and shape setting the wire 296 to change coil direction from a first coil direction (e.g. right-handed coil) of the anchoring portion to a second coil direction (e.g., left-handed coil) of the head 297. The wire forming the neck 289 may remain between the OD 333 and ID 332 of the helix. The neck 289 may have a height 336 (FIG. 16G) that is greater than or equal to the anchor point thickness 335 (FIG. 16H), optionally greater than or equal to the anchor point thickness 335 plus the guidewire thickness. The corkscrew mount 291 may have a total length 337 in a range of 8 to 12 mm (e.g., about 10.4 mm).

The penetrating tip pillar 280 is arranged to be advanced over the guidewire 292 and partially through the lumen 298 of the corkscrew mount 291 to add strength to the anchor system 290 and prevent it from decoupling from tissue. As mentioned, the penetrating tip pillar 280 may have features of the anchor described earlier and shown in FIGS. 9A to 11B some of which are repeated here. The pillar 280 may be a hypodermic tube with a lumen 286 and a sharpened tip 282 (e.g., bevel tip). The hypodermic tube may be connected to a flange 281 that prevents the hypodermic tube from passing fully though the lumen of the corkscrew head 297. Optionally the flange may have a coupling mechanism (e.g. threading, click, bayonet coupling) that couples it to the corkscrew head 297. The hypodermic tube may have an outer diameter (e.g., less than 2.25 mm, in a range of about 1.3 mm to 2.25 mm) that is smaller than the lumen 298 of the corkscrew mount allowing it to pass through the lumen 298 of the corkscrew mount 291. The penetrating tip pillar 280 may be advanced through the outer anchor catheter 293 by pushing it with the inner anchor catheter 294. For example, the inner anchor catheter 294 may have a lumen defined by a wall and the wall may engage with the flange 281. The inner anchor catheter 294 may have a pushrod slidably contained in its lumen and pushable from its proximal end. The pushrod may be used to advance the deployable barb into the tissue.

The deployable barbs 283 may have features of the deployable barbs or anchoring feature 283 described above, such as a preformed, thin gauge (e.g., 0.46 mm), elastic wire (e.g., Nitionl, stainless steel), with sharpened tips that deploys transverse to the axis of the lumen 298. The deployable barbs 283 may further be shaped to deploy radially outward from the lumen 298 and bend proximally to apply a compressive force to the tissue and/or to prevent rotation of the corkscrew so it stays engaged in the tissue. The barbs 283 may have multiple bends. The barbs 283 may have two or more wires that deploy from opposite sides or radially different sides of the pillar. The barbs may extend out of the lumen 286 through the bevel-tip opening 282 of the pillar. Alternatively, the pillar may have side holes through which the barbs are deployed.

In an example shown in FIG. 16H, a corkscrew anchor system may include a corkscrew anchor mount 291 and deployable barbs 283 without the pillar. In this example a pillar may be used to deploy the barbs 283 from within the corkscrew anchor mount 291 and then the pillar may be removed once the barbs are deployed.

The outer anchor catheter 293 has an elongated tubular structure having a central lumen and a coupling at its distal end for engaging the corkscrew head coupling 330. The outer anchor catheter 293 may have an outer diameter in a range of 2.5 to 5 mm (e.g., about 4.3 mm) and an inner diameter in a range of 2 to 4.5 mm (e.g., about 3.8 mm). The tubular structure may be made from a polymer such as Peebax and may have a wire reinforcement to facilitate transmission of torque from the proximal end to the distal end when turning to insert the corkscrew 296 or to decouple from the corkscrew coupling 330. The outer anchor catheter 293 may have a handle on its proximal end, which may be used to torque the elongated tubular structure. The central lumen of the elongated tubular structure may pass through the handle.

The inner anchor catheter 294 has an elongated tubular structure sized (e.g., an OD in a range of 2 to 4.5 mm) to slide through the central lumen of the outer anchor catheter, an inner anchor catheter lumen (e.g., with an ID in a range of 1.5 to 3.5 mm) for slidable passage of the pillar 280 and push rod, and optionally a handle. The inner anchor catheter lumen may pass through the handle.

A method of implanting the corkscrew anchor system 290 may involve inserting one or more anchors (e.g., 2, 3, 4) through an anchor point on a prosthetic valve and into native tissue to hold the prosthetic valve to the tissue in a stable manner that does not pull out under forces applied to the tissue or prosthetic for a suitable duration, for example a duration that allows tissue ingrowth to couple the prosthetic to the native tissue. The anchors may be delivered through anchor delivery catheter(s), which may be delivered through a valve delivery catheter. A process of delivering and implanting the tissue anchors 290, as shown in a flow chart of FIG. 16J, may include the following steps:
1. In step 1, 341 transvascular access to the left atrium is achieved (e.g. with transseptal catheterization involving delivery of a catheter through the patient's vasculature gain access to the left atrium after crossing the interatrial septum from the right atrium) the prosthetic valve is unsheathed and positioned in the desired implant position on native tissue, and the prosthetic valve remains connected to an arm mount shaft 302, anchor guidewires 292 (e.g., two guidewires), and optionally a posterior delivery member 301 (FIG. 16I). FIG. 16A is a simplified figure showing an anchor delivery guidewire 292 passed through an anchor point 215 on an arm 210 with a loop 295 formed in the guidewire distal to the arm 210. This step may include the following sub steps:
   i) The guidewire 292 is temporarily attached to a prosthetic valve anchor point 215, 201, 202 before delivering valve to the patient's heart through a delivery sheath 220. Optionally, a plurality of guidewires 292 are used to attach to a plurality of anchor points. In one example, the prosthetic valve has two anchor points 215, one on each of two arms 210. (FIG. 16I)
   ii) The delivery sheath 220 is advanced to the patient's heart 51 to prepare for implanting the prosthetic valve 100 through the delivery sheath 220. In one example the delivery sheath is passed through the patient's vena cava to the right atrium, across the atrial septum to the left atrium 57 and to the native mitral valve 54 (FIG. 16I). In a second example the delivery sheath is passed through the apex of patient's heart into the left ventricle and to the mitral valve.
   iii) The prosthetic valve 100, coupled to the guidewire 292, is advanced distally from the delivery sheath 220 into the patient's heart.
2. In step 2, 342 (FIG. 16J, 16B) the corkscrew anchor mount 291 is advanced over the guidewire 292 and implanted into tissue (e.g. a trigone 52, 53). This step may include the following sub steps:
   i) The corkscrew anchor mount 291, coupled to the outer anchor catheter 293 is advanced over the guidewire 292 to the anchor point 215. Optionally the corkscrew anchor mount is temporarily held to the outer anchor catheter 293 via a detachable mechanism such as a threaded coupling 330.
   ii) The corkscrew anchor mount 291 is turned to drive the corkscrew through the valve anchor point 215 into native tissue (e.g., trigones 52, 53), for example by rotating the outer anchor catheter in a first direction (e.g. clockwise). Haptic feedback may be felt through the outer anchor catheter as the corkscrew anchor mount is advanced and the neck 289 passes through the anchor point 215 indicating to the user that the corkscrew anchor mount 291 is fully inserted.
3. In step 3, 343 (FIG. 16J, 16C) the guidewire 292 is removed leaving the corkscrew anchor mount 291 positioned through the anchor point 215 and embedded in tissue 52, 53, with the arm 210 held between the corkscrew head flange 299 and the tissue, and wherein the corkscrew anchor mount remains coupled to the outer anchor catheter 293. This step may include the following sub steps:
   i) The guidewire 292 shown in previous steps (FIG. 16A, 16B) is pulled out by pulling on the proximal end of the guidewire, and optionally holding the outer anchor catheter 293 relatively still to prevent the guidewire from tugging on the anchor point. Tension causes loop 295 to unwind and be pulled through the anchor point. The corkscrew mount 291 is left inserted into tissue through the anchor point and coupled to the outer anchor catheter, which provides a delivery lumen for the subsequent steps.
4. In step 4, 344 (FIG. 16J, 16D) the tubular pillar 280 is inserted through the corkscrew anchor mount 291 and the deployable barbs 283 are deployed. This step may include the following sub steps:
   i) The tubular pillar 280 is temporarily connected to an inner anchor catheter 294 (e.g., via a threaded mating, form fit) and advanced through the outer anchor catheter 293.
   ii) The tubular pillar 280 with a penetrating tip 282 is advanced into the native tissue through the lumen of the corkscrew mount 291. A little tension may be applied to the outer anchor catheter 293 to hold the tissue while the penetrating tip 282 is advanced into it to prevent tissue tenting. Optionally, the handle of the inner anchor catheter and the handle of the outer anchor catheter engage with one another to facilitate this step (not shown). For example, an indicator (e.g., a haptic click or a visual alignment of markers) provided on the inner and outer anchor catheter handles may be used to indicate when the distal penetrating tip 282 of the tubular pillar 280 is aligned with the corkscrew head 297, and an actuation mechanism in the handles may cause the penetrating tip 282 to be advanced a predefined distance while the corkscrew is held. Optionally the actuation mechanism has a spring-loaded plunger that applies a quick and limited amount of force to advance the pillar through the corkscrew.
   iii) The barbs 283 are deployed from the tubular pillar 280, e.g., by advancing push rod 287 in the lumen of the inner anchor catheter 294. FIG. 16D
5. In step 5, 345 (FIG. 16J, 16E, 16F) The tubular pillar 280 is decoupled from the inner anchor catheter (e.g. by rotating the inner anchor catheter to unscrew the coupling or pulling on it to release a form fit coupling); the corkscrew 291 is decoupled from the outer anchor catheter 293 (e.g., by unscrewing); optionally both are decoupled simultaneously (FIG. 16E).
   i) The inner anchor catheter and outer anchor catheter are removed from the body and the anchor system 290 remains in tissue with flanges on opposite side of valve anchor point holding the prosthetic valve anchor point firmly to tissue (FIG. 16F).
6. If multiple anchors are needed, a guidewire for each anchor point is preconnected to the anchor point prior to advancing the prosthetic valve into the patient, and steps 2 to 5 are repeated for each anchor.

### Seal

The structural frame 120, arm(s) 210, or leg(s) 190 may be covered at least partly in a flexible membrane 175, which may be impenetrable to blood flow. The membrane may be made from a biocompatible, flexible material such as polyurethane, which may be adhered or sewn to the structural frame, arm(s) or leg(s). The membrane 175 may be doped with an agent to enhance biological acceptance such as anti-coagulants. The flexible membrane may be treated to have hydrophobic properties, at least in some locations on the prosthetic valve 100 such as areas intended to not contact tissue such as the ventricle section, which may facilitate flow of blood or reduce a risk of clotting or tissue ingrowth in this area. A hydrophobic treatment may include a hydrophobic micropatterned surface molded to the membrane or a lubricious coating. Alternatively, a membrane 175 may be dip coated or applied to one side of a fabric layer 176 that is sewn or adhered to the structural frame, arms or leg(s).

The structural frame 120, arm(s) 210, or leg(s) 190 may be covered at least partly in a fabric such as felt or a woven fabric, which may facilitate tissue ingrowth to advantageously hold the prosthetic valve 100 securely to native tissue to improve longevity. The fabric layer 176 may be attached to areas of the prosthetic valve 100 intended to securely contact native tissue such as the external side 108 of the atrial section 121 or annulus section 122.

The fabric 176 may extend beyond the structural frame 120 in a superior direction 104 from the atrial section 121 (FIG. 7B), which may spread contact force or reduce risk of tissue injury. The fabric 176 may have a notch 177 (FIG. 7B), which may be used to align the prosthetic valve with an anatomical structure or a center line of the native mitral valve.

### Delivery

Before delivering the prosthetic valve into a patient's heart a sizing tool may be delivered that accurately measures key dimensions of the patient's native mitral valve or heart structures, such as the native mitral annulus major axis, minor axis, distance between trigones, distance between commissures. Additionally or alternatively, medical imaging such as CT scans may be used to measure key dimensions of the patient's native mitral valve or heart structures. The measured dimensions may be used to select an appropriately sized prosthetic valve or to adjust dimensions of the prosthetic valve (e.g., such as arm length) before implanting.

The implant procedure may be performed through an endovascular intervention through a delivery sheath that may be 28 Fr or less (e.g., less than 27 FR, less than 26 FR, less than 25 FR, less than 24 FR, 23 FR or less, 22 FR or less, 21 FR or less, 20 FR or less, 19 FR or less).

Optionally, chordae tendinea connected to the posterior leaflet may be severed before or after implanting the prosthetic valve, for example if the patient's anatomy includes chordae that are particularly short or if the papillary muscles are positioned in such a way (e.g., further toward the anterior leaflet) that will cause the chordae connected to the posterior leaflet to be placed under tension or to pull on the papillary muscles when the prosthetic valve is implanted or if they inhibit the native posterior leaflet from being moved away from the mitral orifice or inhibit proper positioning of the prosthetic valve. A procedure may involve always severing the chordae before implanting a prosthetic valve. However, it is expected that the design disclosed herein may not require severing the chordae in any situation, which beneficially may save procedure time and may have functional benefits. For example, preserving the chordae may allow use of them to place the anchoring leg between to help center the valve; the chordae may help to hold the native posterior leaflet in an elongated state against the posterior of the frame, which may contribute to preventing perivalvular leakage; the chordae may contribute to stabilizing or orienting the prosthetic valve in a functioning position relative to the native structures; the chordae may contribute to maintaining a space between the posterior of the ventricular section and the ventricle wall, which may promote blood flow in this space to avoid stagnation that can result in thrombosis.

In a delivery configuration with the prosthetic valve 100 collapsed and contained by a tube such as a delivery sheath 220, the leg 190, or optionally multiple legs, may be folded toward the distal end of the delivery sheath as shown in FIG. 12A. Alternatively, the leg(s) 190 may be directed toward the proximal end of the delivery sheath. When the prosthetic valve 100 is partly advanced so the leg 190 is no longer constrained in the delivery sheath 220, the unconstrained leg 190 may spring radially outward from the prosthetic valve at a partly deployed leg angle 198 as shown in FIG. 12B. This partly deployed leg angle 198 may be facilitated by the spacing between the first and second leg ends 191, 192 (see FIG. 5A), which may be decreased when the prosthetic valve 100 is in a delivery configuration.

When the prosthetic valve 100 is fully deployed from the delivery tube, e.g., delivery sheath 220, and the structural frame 120 is expanded, the leg 190 may bend back toward the structural frame such that the fully deployed leg angle 199 is less than the partly deployed leg angle 198 as shown in FIG. 12C. This leg angle feature may facilitate delivery of the prosthetic valve as it is clearly presented protruding from the central axis of the delivery sheath 220 when partially deployed, and the delivery sheath may be rotated to aim the leg 190 between chordae bundles to correctly orient the prosthetic valve 100 with respect to the native posterior mitral valve.

Alternatively or additionally, a prosthetic valve may have additional legs 320 and as shown in the sequence of deployment in FIGS. 13A to 13D, the additional legs 320 may be contained in the delivery sheath 220 in a delivery configuration that is folded in a proximal direction while the primary leg 190 is folded in a distal direction, which may facilitate deployment of the primary leg first and deployment of the additional legs after further retraction of the delivery sheath in a proximal direction. Deployment of the primary leg 190 first may facilitate positioning of the primary leg centered on the posterior of the annulus, in the center of the P2 scallop, and between the chordae tendineae 65 of the medial 67 and lateral 66 papillary muscles, before the additional legs 320 are deployed, which may further facilitate engagement of the prosthetic valve in proper alignment with the native annulus.

The prosthetic valve 100 may be delivered surgically or endovascularly with a delivery method illustrated in FIG. 12A, FIG. 12B, and FIG. 12C, which may include the following steps:

A distal end of a delivery sheath 220 may be advanced from a left atrium 57, optionally with a trans-septal approach, through a native mitral valve orifice between a native posterior leaflet 64 and an anterior leaflet 63 (FIG. 12A).

The prosthetic valve 100 may be advanced partly from the delivery sheath 220 by pushing on a prosthetic valve delivery shaft 230 such that the prosthetic valve is still constrained at least in part in a delivery configuration, and the leg 190 is not constrained by the delivery sheath 220, wherein the leg 190 springs radially outward at a partly deployed leg angle 198. For example, the delivery sheath may be retracted to a first position 322 (FIG. 13D) to deploy the leg 190.

The leg 190 may be positioned through a space between chordae of the lateral papillary muscle 66 and chordae of the medial papillary muscle 67, (FIG. 12B).

The prosthetic valve 100 may be pulled in a superior direction 102, which may capture the native posterior leaflet 64 between the leg 190 and rest of the prosthetic valve 100 and may provide an indication that the prosthetic valve is correctly oriented rotationally with respect to the native mitral valve before fully deploying it. The leg 190 positioned through the chordae bundles may also facilitate positioning of the prosthetic valve 100 in a superior inferior direction by preventing further motion in the superior direction as it is pulled. The correct position of the leg 190 through the chordae bundles and behind the native posterior leaflet may be confirmed radiographically from the RO marker 200. The correct position of the prosthetic valve 100 in the superior-inferior direction may not need to be confirmed radiographically. Alternatively, a radiopaque marker on the annulus section 122 may be aligned with the mitral annulus radiographically.

In the case of delivering a prosthetic valve 100 having a primary anchoring leg 190 and two additional legs, the delivery sheath may be retracted more proximally to a second position 321 that deploys the two additional legs 320, as shown in FIG. 13D. The two additional legs may be positioned on the inferior side of the mitral annulus 13 and may press into a portion of the native P1 and P3 scallops such that the native P1 and P3 scallops are compressed between the additional legs 320 and the native ventricle or annulus tissue, or alternatively the additional legs 320 may capture the native P1 and P3 scallops such that said scallops are positioned between the additional legs 320 and the structural frame. Alternatively, the two additional legs may each have a protrusion superior to the annulus as well as inferior to the annulus allowing them to clamp onto the annulus in the P1 and P2 regions. Alternatively, the two additional legs may be positioned in the commissures, one between the P1 an P2 scallop, and the second between the P3 and P2 scallop.

The delivery sheath 220 is retracted to fully release the prosthetic valve 100 from the constraining lumen of the delivery sheath allowing it to transition to its deployed configuration (FIG. 12C, which shows only a side profile of the prosthetic valve 100 for simplicity). The transition causes the first 191 and second 192 ends of the leg 190 to spread apart as the structural frame 120 uncoils and expands, which in turn causes the leg 190 to move toward the structural frame and the partly deployed leg angle 198 decreases to the fully deployed leg angle, thus capturing the native posterior leaflet between the leg 190 and structural frame while straddling the chordae bundles. The atrial section 121 also deploys into the atrium 57 flaring out from the annulus section 122 to create a flange which helps the prosthetic valve self-seat into a correct position with the annulus section aligned with the posterior annulus, the atrial section 121 conformed to the wall of the left atrium, and the ventricle section 123 flared radially from the annulus section into the left ventricle.

Once the prosthetic valve 100 is fully deployed from the delivery sheath 220 tissue anchors 280 may be inserted into anchor points on arm(s) 215 (FIG. 8A) and/or anchor points from an atrial petal to leg 202 (FIG. 12C). The prosthetic valve delivery shaft 230 may be releasably connected to the prosthetic valve, optionally to a plurality of connection points such as the delivery interface holes 138, enabling both a pushing and a pulling motion to maneuver the prosthetic valve 100. An actuator in a handle of the delivery shaft 230 may release the prosthetic valve, for example with threads threaded through loops of the prosthetic valve (e.g., atrial petal apexes 133). The prosthetic valve delivery shaft 230 may be an anchor delivery catheter 240 that both manipulates the prosthetic valve 100 within a delivery sheath 220 and interfaces with a tissue anchor 280, such as a tissue anchor provided engaged with an anchor point on an atrial petal 202 ready to be advanced through tissue. A thread threaded through loops of the prosthetic valve (e.g., atrial petal apexes 133) may be under tension while the anchor delivery catheter 230 is under compression as the anchor 280 is pressed into the tissue. Alternatively, tissue anchors may be delivered through a delivery sheath 220 after the prosthetic valve 100 is deployed into position. The prosthetic valve delivery shaft 230 may be removed from the delivery shaft 220 before a tissue anchor is advanced while connected to an anchor delivery catheter 240 and directed through an anchor point 215, 202.

Alternatively, anchors may be inserted before deploying the prosthetic valve. The anchors may be inserted into trigones and be connected to a suture or rail that is accessible through the delivery system and may pass through anchoring features of the arms. Then the arms may be deployed and loaded along the suture or rail to the anchor. A cap or nut or containing element may connect or hold the arm (e.g., anchoring feature of arm) to the anchor. The cap or nut or containing feature may be delivered through the delivery system, for example, over the suture or rail.

### Arm Mount Delivery System

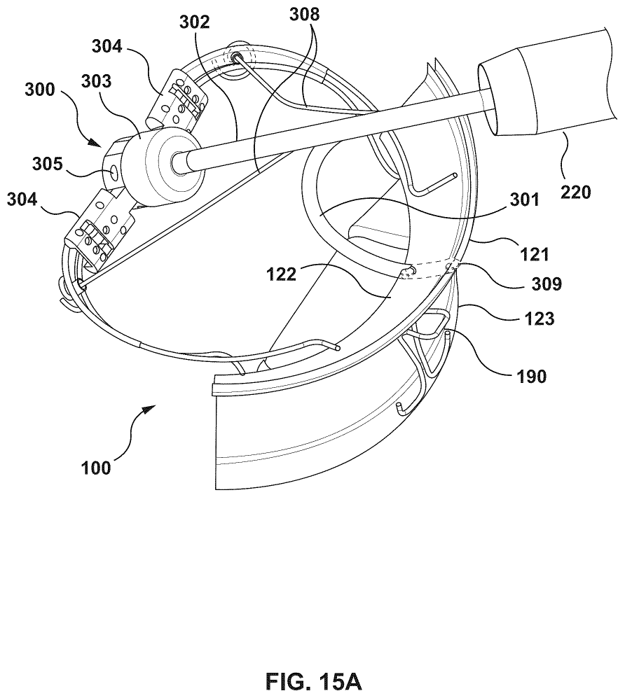

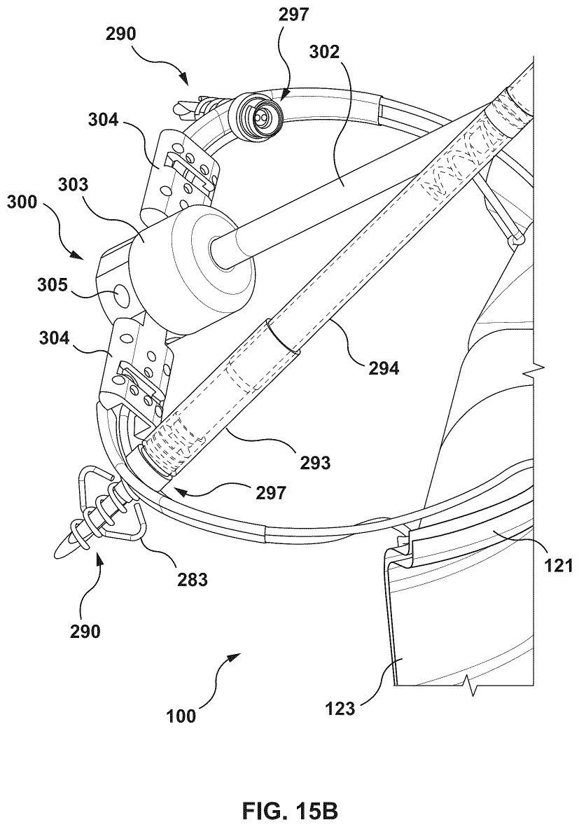

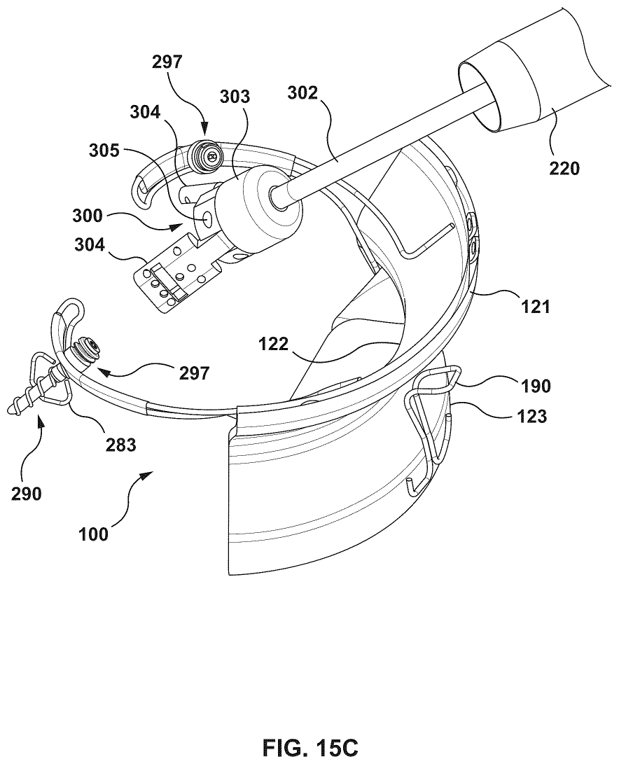

Another embodiment of a delivery system for transvascular delivery of a prosthetic mitral valve 100 may have an elongate tubular delivery sheath 220 with a lumen that is controllably deflectable (e.g., deflectable in one or two planes) by manipulating an actuator on its handle. The handle may have an access port to the lumen and may have a hemostasis valve. The delivery system may also have a dilator that fits in the lumen of the delivery sheath with a tapered distal end protruding from the distal end of the delivery sheath when fully inserted into the delivery sheath. The dilator may have a hub that mates onto the access port of the handle of the delivery sheath. The dilator may have a central lumen with a hemostasis valve for advancing over a guidewire, an atrial septum puncture catheter, a left atrium access catheter or a combination thereof. A prosthetic valve manipulation system is adapted to hold and control position of the prosthetic valve and is adapted to be positioned and/or pass through the lumen of the delivery sheath. As shown in FIG. 15A prosthetic valve manipulation system may include and arm mount 300 connected to an arm mount shaft 302 that is connected to a prosthetic valve manipulation system handle; the system may further include a posterior delivery member 301 that may be connected to the prosthetic valve manipulation system handle. The delivery system may also include two anchor delivery guidewires that may be advanced through the delivery sheath lumen along with the prosthetic valve manipulation system. Tissue anchors may be advanced over the anchor delivery guidewires and manipulated to be inserted and deployed using tissue anchor catheters, which also may be advanced through the delivery sheath lumen.

The prosthetic valve is compressed into a delivery state for example by folding or rolling it into a somewhat cylindrical shape that fits in a lumen of the delivery sheath 220, as shown in FIG. 13A.

Prior to inserting the prosthetic valve 100 into the delivery sheath 220, the prosthetic valve may be temporarily connected to a posterior delivery member 301 at a posterior region of the atrial section 121 of the structural frame 120. The posterior delivery member 301 may be an elastically resilient tube (e.g., Nitinol) having a lumen through which a thread 309 (e.g., Kevlar) is positioned. The thread 309 may loop around a piece of the structural frame in the atrial 121 or annulus 122 section and both ends or at least one end of the thread may pass through the lumen to the proximal end of the delivery system where it is fastened to a posterior delivery member release mechanism. To release the structural frame from the posterior delivery member the posterior delivery member release mechanism may be loosened to release the thread. The posterior delivery member 301 may pass through the delivery sheath to the proximal end of the delivery system where it may be controllable independently from the arm mount shaft 302, for example pushed or pulled from its proximal end, for example with a posterior delivery member translation actuator in a handle. The distal end of the posterior delivery member may have a mating feature, such as an indent, that accepts the strut of the structural frame.

Prior to inserting the prosthetic valve 100 into the delivery sheath 220, anchor guidewires 308, (e.g., two, or one for each arm anchor point) may be temporarily connected to the anchor points 215 on the arms and pass through the lumen in the delivery sheath 220. The guidewires 308 may be connected to the anchor points 215 for example by passing through the aperture of the anchor points and having a preformed bend or loop on the distal side of the anchor point. The guidewires may be removed with a small pull force applied to the proximal end of the anchor guidewires, for example once the anchors are at least partially inserted.

Prior to inserting the prosthetic valve 100 into the delivery sheath 220, the tips 219 of each arm 210 may be temporarily connected to an arm mount 300, which is connected to an arm mount shaft 302. The arm mount 300 has body 303 that is connected to the arm mount shaft 302, and two hinged wings 303 that are connected with hinges 305 to the body 303. Each of the two hinged wings 303 temporarily connect to the tips 219 of each arm during delivery. The arm mount 300 has a delivery state, wherein the two hinged wings 303 are folded together distal to the body, and wherein the arm mount can slidably fit in the delivery sheath lumen 221. The arm mount 300 has a deployed state, wherein the two hinged wings are spread open, approximately perpendicular (e.g., within a range of 75 to 90 degrees) to the axis of the arm mount body 303. The hinged wings may be transitioned from the delivery state to the deployed state by an actuated mechanism. For example, a deployment rod 306 may be contained in a lumen 307 in the arm mount shaft, wherein an actuation motion such as pushing or pulling the proximal end of the deployment rod translates to the distal end of the deployment rod where it acts on the hinged wings to pivot them on their hinges; or rotating the proximal end of the deployment rod turns a threaded distal end of the rod which engages the hinged wings to pivot on their hinges. Alternatively, a spring may push the hinged wings into a deployed state when the arm mount is released from a delivery sheath or when tension is released from the deployment rod. The deployment rod may be actuated at its proximal end for example by an arm mount deployment actuator on a delivery system handle. The arm mount shaft may be controllably deflectable, for example in one or two planes of deflection, and may be controlled by an arm mount shaft deflection actuator on a handle of the delivery system.

The prosthetic valve may be inserted into the distal end of the delivery sheath 220 by pulling the arms into the sheath first, for example by rolling or folding the prosthetic valve into a collapsed delivery state and pulling the proximal end of the arm mount shaft that is connected to the tips of the arms via the arm mount 300. With the arms first inserted into the delivery sheath the rest of the prosthetic valve may be inserted by pulling the arm mount shaft 302 and/or the posterior delivery member 301 and/or pushing the prosthetic into the sheath. The anchoring leg 190 may be elastically bent in a distal direction as the prosthetic valve is pulled into the delivery sheath allowing it to be the first part of the prosthetic to be exposed from the sheath during delivery. FIG. 13A shows the prosthetic valve 100 inserted into a distal region of a delivery sheath 220. Optionally, a folding tool may be used to help fold, or roll the prosthetic valve and insert it into the delivery sheath.

### Deployment

The prosthetic valve may be implanted using a transcatheter method, wherein an access catheter is positioned through a patient's vasculature, for example from a femoral vein through the vena cava and across the atrial septum, to the left atrium and the delivery sheath 220 is passed through the access catheter and out the distal end into the left atrium 57 (FIG. 12A).

The delivery sheath may be controllably deflectable in at least one plane of deflection, which may be controlled by a deflection actuator 222 on a handle 223 of the delivery sheath. The delivery sheath 220 may be retracted with respect to the prosthetic valve, for example by pushing the delivery shaft 230 relative to the delivery sheath. This action may be done in a controlled manner with a linear translation actuator 231 on a handle that may advance the delivery shaft 230 by one or more predefined amounts according to one or more stages of deployment. The delivery sheath 220 may be retracted to expose the leg 190 as shown in FIG. 12A or FIG. 12B. The prosthetic valve may be loaded into the delivery sheath with the leg radially aligned relative to (e.g., in the same direction as, or at an angle to) the plane and/or direction of deflection of the delivery sheath for example, by aligning the leg with a visual marker on the delivery sheath that corresponds to a desired deployment alignment relative to the plane and/or direction of deflection. The leg 190 may have a radiopaque or echogenic marker so it can be imaged with medical imaging equipment. The distal region of the delivery sheath may be advanced into the native mitral valve and the leg 190 may be positioned between the chordae tendineae 65 of the lateral 66 and medial 67 papillary muscles and centered to the P2 scallop of the posterior leaflet 64. The delivery sheath may be moved in a direction toward the P2 scallop to ensure the native P2 scallop is captured between the leg 190 and the frame. The prosthetic valve may be pulled in a superior direction causing the apex 193 to engage with the AV groove behind the P2 scallop.

The delivery sheath 220 may be further retracted relative to the delivery shaft 230, which further deploys the prosthetic valve connected to the arm mount, posterior delivery member, and anchor guidewires. The arm mount may be connected to the arms, for example the distal end of each arm or somewhere along the length of the arms. The arm mount 300 may be actuated to splay out the arm mount wings 403. The prosthetic valve may be manipulated to orient it relative to the native mitral annulus. For example, superior tension may be applied to the posterior of the prosthetic valve from the posterior delivery member while the more anterior aspect of the prosthetic is pushed inferiorly by manipulating the arm mount shaft. Adjustments in position may also be made by adjusting deflection or rotation of the delivery sheath 220 and/or deflection or rotation of the arm mount shaft 302.

The arm mount position may be manipulated by deflecting the arm mount shaft, deflection of the delivery sheath, and/or torque of the arm mount shaft or a combination thereof, to align the arm anchor points with a target anchor point in native tissue such as native trigones, preferably not on the aortic curtain. Also the arm mount, connected to the valve so that the center of the arm mount aligns with the line of symmetry of the prosthetic valve, may be centered along a line of symmetry of the native valve, or at least within 5 mm (e.g., within 4 mm, within 3 mm, withing 2 mm) of the line of symmetry.

The arm mount and/or the posterior delivery member may be manipulated to apply pressure between the structural frame against the posterior native valve. When this is pressure is applied a user may see bowing of posterior delivery member which may indicate appropriate back pressure.

Optionally deployment of trigone barbs into native tissue from or through the arms may be done to temporarily hold the arms in place relative to the native valve with the pressure being applied to the posterior.

Anchors may be advanced over anchor guide wires and through arm anchor points into native tissue (e.g. trigones), for example, using various embodiments of tissue anchors and methods of deployment described herein. (FIG. 15B)

Pressure on posterior delivery member may be released from the posterior delivery member and alignment of the prosthetic may be checked before releasing anchor guidewires. If position is satisfactory the anchor guides may be released from the prosthetic valve.

The arm mount wings maybe released from the arms, e.g. suture or release mechanism (FIG. 15C). The delivery system may be removed.

### Adjustable Arms

In some embodiments the arms 210 may be adjustable in situ where adjustability may be in arc length, radius of curvature, or with an adjustable limit to flexion. By adjusting the arm length, radius of curvature, tension, compression, or flexion limit, the prosthetic may be fine-tuned in situ to fit the patient's heart properly; or to adjust the arm properties after arms have been anchored to the tissue to adjust performance of the valve; or to adjust the dilation of the mitral annulus to improve coaptation of the anterior leaflet and the prosthetic posterior leaflet.

Adjustability in arc length may be accomplished with an adjusting mechanism in each arm wherein each arm is made of a first section and a second section that move relative to one another and lock in place with a locking mechanism. The first and second sections of the arms may be for example sliding rails or telescopic. The adjusting mechanism may be for example a threaded component, a ratchetting system, or a pull string. The locking mechanism may be for example, sawtooth interfaces or a threaded component.

Radius of curvature may be adjusted, for example by adjusting the length of an anterior arm bridge 209, which may be a thread connecting the distal ends of each arm (FIG. 8D). The anterior arm bridge thread can be a flexible Kevlar thread that, when taut prevents the arm ends from moving apart any further, establishing a maximum prosthetic circumference. The thread, being collapsible, may freely allow the ends of the arms to move toward one another. This may allow the prosthetic to move and deform along with the native valve structures, increasing and decreasing in circumference, yet limit the prosthetic circumference to a maximum circumference so the annulus does not over dilate over time. This may prevent reoccurrence of mitral regurgitation due to annulus dilation and spreading of the anterior and posterior leaflets. In other words, altering the length of an anterior arm bridge in situ may adjust the radius of curvature of the arms, the circumference of the prosthetic and adjust where the maximum limit to arm flexion.

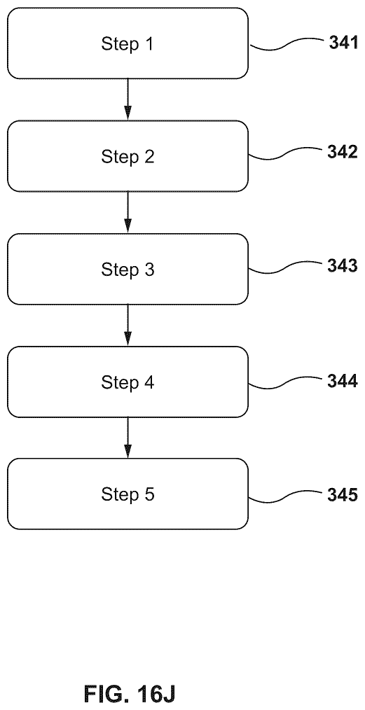

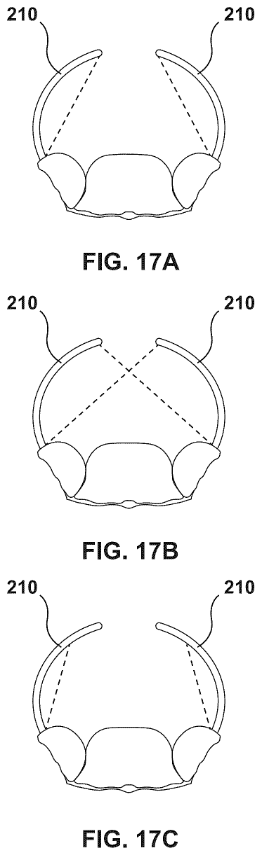

In situ arm adjustability may alternatively be accomplished with Kevlar thread connecting a part (e.g., distal tip) of each arm to a different part (e.g., a portion of the structural frame of the annulus section or atrial section) of the prosthetic as shown in FIGS. 17A to 17C. The thread(s) may be adjustable in length in situ by being connected at a distal end to a first location on the prosthetic, slidably passing through a hole or loop at a second location on the prosthetic a distance from the first location, and passing through a lumen of the delivery system where the proximal end of the thread(s) maybe pulled to shorten the distance between the first and second locations, or released to lengthen the distance. Once the desired length is achieved the thread may be fastened at its maximum length, e.g. crimped, glued, clamped, tied, and the remaining length of thread may be cut and removed out of the delivery system.

### Optimizing Flow Path

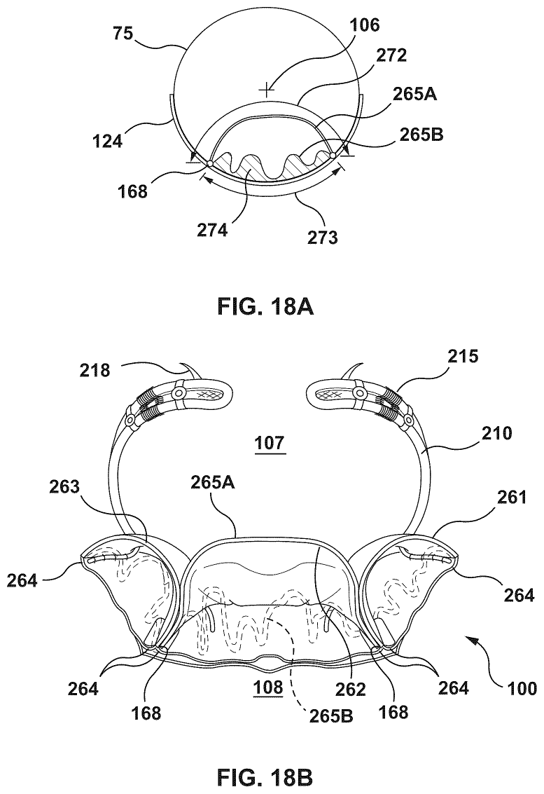

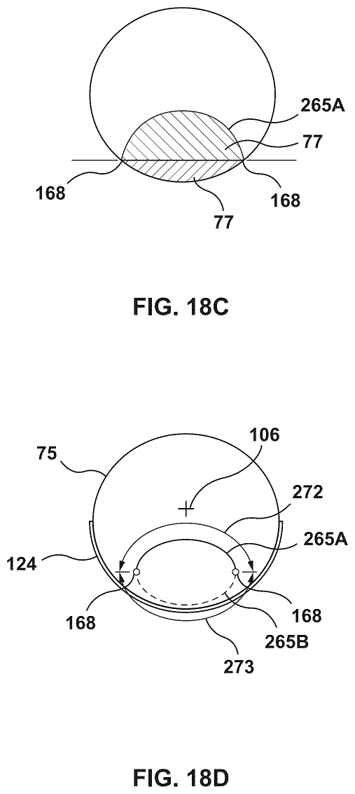

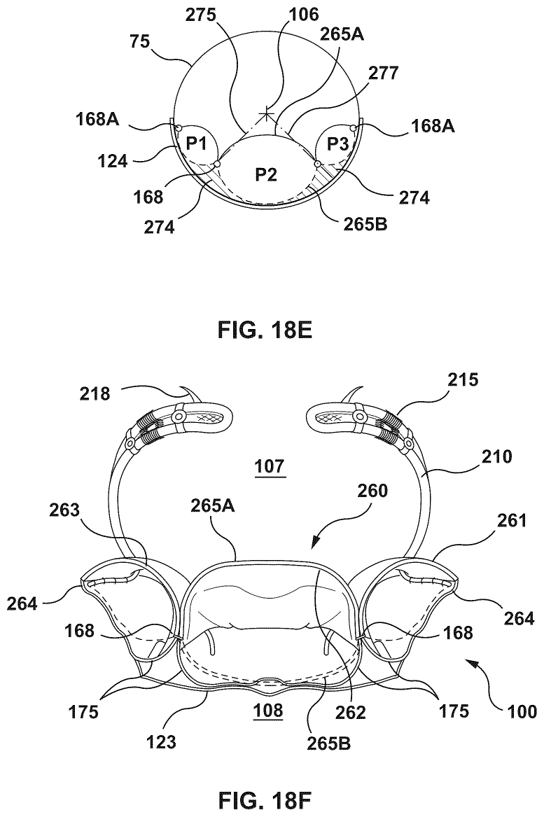

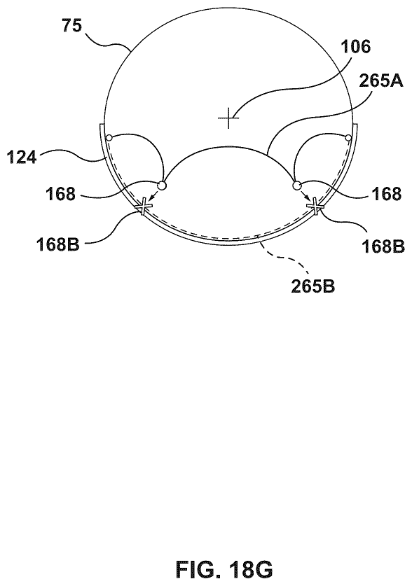

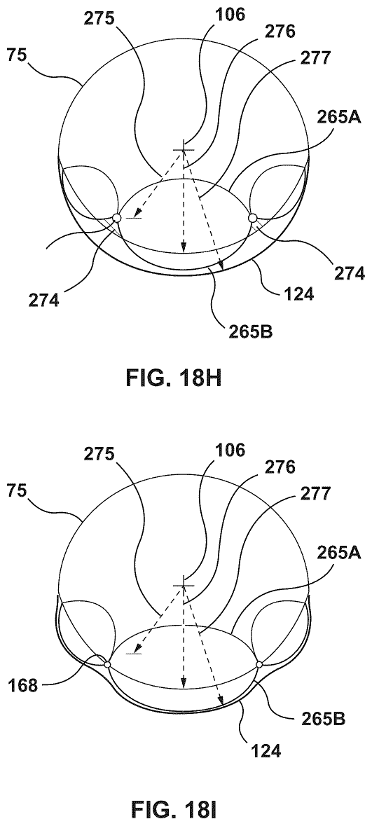

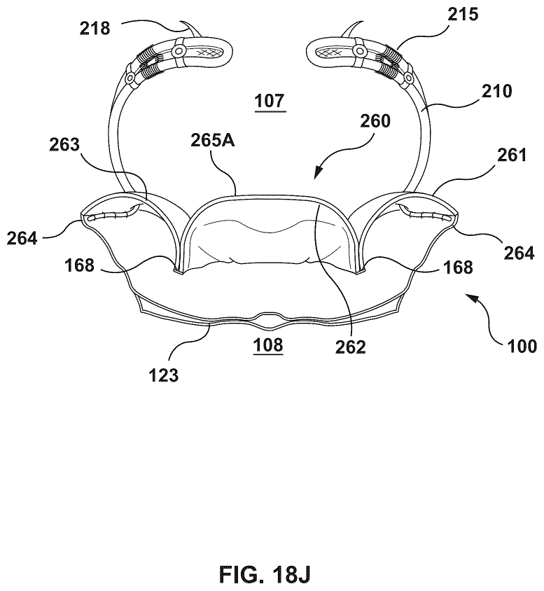

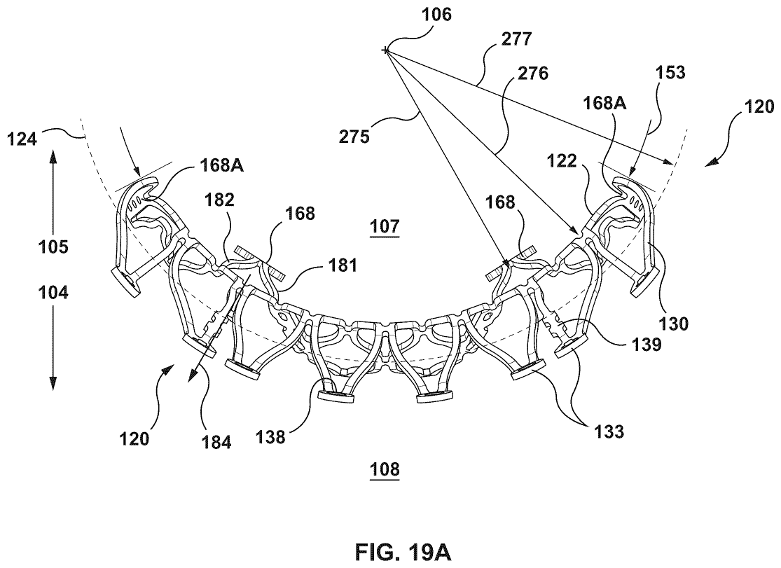

During systole (i.e., contraction of the ventricles), the higher pressure in the left ventricle compared to the left atrium causes blood to fill the space defined by the scallops and structural frame causing the scallops to billow and coapt against one another and the native anterior leaflet in the closed valve state. During diastole, the reverse pressure difference causes the scallops to be emptied of blood and blood flowing from the left atrium pushes the scallops away from the central axis 106, in this case toward or against the ventricle section of the structural frame in an open valve state, thus opening the mitral orifice so blood can flow through the mitral orifice and into the left ventricle through a flow path. The flow path 75 may be considered as a cylindrical projection of the open orifice in the annulus during diastole, projected into the left ventricle. FIG. 18A shows a cross section of a flow path 75 represented as a circle for simplicity, however, a flow path cross section may be oval, oblong or in general shaped like the native annulus orifice and project into the ventricle. An optimum design may be one in which the flow path is left as unobstructed as possible (e.g. mimicking the native flow path), which is expected to allow the heart to perform optimally, for example obtain appropriate pressure gradients and flow rates. A prosthetic mitral valve design may include features that beneficially allow the leaflet scallops to move out of the flow path as much as possible in an open valve state. For example, a prosthetic mitral valve may include a sufficient space for occupying each prosthetic leaflet scallop in the open valve state so the scallops can move out of the flow path and into the space. For example, these spaces may be created in the prosthetic valve by means of shaping the ventricle section of the structural frame and positioning the leaflet connection frames (e.g., commissures 168 between P1 and P2, and between P2 and P3) as a function of surface area of the scallops.

To illustrate this concept, an example in which a prosthetic valve has insufficient space for a scallop to move into in an open valve state, is first described followed by examples in which a prosthetic valve has improved space for a scallop to move into to create a more open flow path. A bottom view of a prosthetic valve with a leaflet scallop in a closed state (solid lines) and open state (dashed lines) is shown FIG. 18B and a simplified 2-D representation thereof is shown in FIG. 18A, where the edge of a scallop in the closed state 265A has an arc length 272 that is longer than an arc length 273 of the structural frame 123 between the two P2 scallop commissures 168. The flow path is represented in the 2-D figure as a circle 75. Arc length 273 may be defined as the arc length (or area in 3-D) of a structure (e.g., structural frame) between the commissures 168 that defines the space into which the adjoined scallop can move into during diastole because the adjoined scallop is a flexible sheet of material fixed to the structural frame at the commissures 168. Only one scallop is shown in FIG. 18A for simplicity. This arrangement with insufficient space for the scallop to move into may cause the scallop in an open valve state 265B to wrinkle, instead of pressing against the structural frame in a smooth unwrinkled manner, to compensate for the difference in length between arc length 272 and arc length 273. In 3-D the extra scallop material in the open state may wrinkle or otherwise be prevented from moving out of the flow path. Furthermore, this scallop behavior may decrease other performance factors such as the ability to open and close smoothly and under appropriate pressure differences, ability to flush, or durability. Another way to illustrate this concept is by comparing volume of a scallop in the closed valve state to volume of the space into which the scallop can move into in the open valve state. FIG. 18C is a simplified 2-D representation of the arrangement in FIG. 18A showing volume of the scallop in the closed valve state 265A, represented by area 76, and volume of the space into which the scallop can move into, represented by area 77, wherein the area 76 is larger than the area 77 (or likewise the volume represented by area 76 is larger than the volume represented by area 77), which impedes the scallop from fully inverting (i.e., moving into the space 77) in the open valve state.

An alternative design having improved space for a scallop to move into, is illustrated in a simplified 2-D representation in FIG. 18D. Compared to the example shown in FIGS. 18A, 18B, and 18C the positions of the P2 commissures 168 are shifted from the curved plane 124 formed by the ventricle section 123 toward the central axis 106 resulting in the arc length 272 of the scallop in a closed state 265A being shortened and the space into which the scallop can move into in its open state being enlarged, so that the scallop in the open state 265B has sufficient space to move into resulting in an open state that is smooth and unwrinkled or at least less wrinkled. In such an arrangement arc length 272 is much closer (e.g., no more than 5% greater than) to the arc length 273, preferably less than or equal to arc length 273. This arrangement moves more scallop material out of the flow path and also allows the leaflet to function better. FIG. 18E is a 2-D simplified representation of the concept shown in FIG. 18D showing the three scallops P1, P2, P3 of the prosthetic posterior mitral leaflet, wherein the P1/P2 and P2/P3 commissures 168 are shifted toward the central axis 106 and the side commissures 168A remain on the curved plane 124 of the ventricle section 123. FIG. 18F is a bottom view of a prosthetic valve corresponding to the simplified version of FIG. 18E. In addition to resolving the wrinkles in the P2 scallop, the P1 and P3 scallops are likewise resolved. The shifting of the position of the commissures 168 toward the central axis 106 decreases the arc length of the P1 and P3 scallops while increasing the space for them to move into so in the open valve state they have a smooth, unwrinkled profile, which further improves the flow path and valve function. The arc length of the P1 and P3 inferior edges in the closed state are substantially equal to (e.g., equal to, no more than 5% greater than) or less than the arc length of the space available for the scallops to move into in the open state. In the 2-D representation of FIG. 18E the area 274 filled with hash marks represents material of the prosthetic valve in the open state that remains in the native flow path 75. Although some material 274 may remain in the flow path 75, it may be less than the amount of material left in the flow path in the arrangement of FIG. 18A, while also benefiting from improved function. The position of the P1/P2 and P2/P3 commissures 168 may be at a distance 275 from the central axis 106 of the native annulus that is less than the distance 276 from the annulus section 122 during systole to the central axis 106 when the prosthetic valve is implanted. For example, distance 275 may be in a range of 70% to 90% (e.g., about 80%, in a range of 75% to 85%) of the distance 276. The position of the P1/P2 and P2/P3 commissures 168 may be at a distance 275 from the central axis 106 of the native annulus that is less than the distance 277 from the curved plane 124 of the ventricle section 123 to the central axis 106 when the prosthetic valve is implanted. For example, distance 275 may be in a range of 70% to 90% (e.g., about 80%, in a range of 75% to 85%) of the distance 277. A prosthetic valve may be arranged such that the curved plane 124 of the ventricle section of the structural frame is aligned with (as shown in FIG. 18G) or exterior to (as shown in FIG. 18H) the flow path 75.

Optionally, as shown in FIG. 18H, the amount of material left in the flow path may be further reduced by shifting the position of the ventricle section of the structural frame 123 exterior to the flow path 75 (i.e., further out of the flow path), while maintaining the relationship between commissure position and scallop dimensions that permits the scallops to fully open without wrinkling, so the position of the commissures 168 can be less toward the central axis 106 resulting in a smaller amount of material left 274 in the flow path 75 as compared to the arrangement in FIG. 18E, wherein the curved plane 124 of the ventricle section of the structural frame is aligned with an edge of the flow path 75. In this example, some material 274 still remains in the flow path 75, however it is less than the amount 274 in FIG. 18E and maintains the benefit of allowing the scallop to fully open with minimal to no wrinkling. In some examples a ratio of distance 275 to distance 276 is greater than or equal to 0.8, and a ratio of distance 277 and distance 276 is greater than or equal to 1.2.

In some examples, as shown in FIG. 18I the curved plane 124 of the ventricle section 123 is further exterior to the flow path (e.g., further from the central axis 106 than the annulus section 122), by an amount that is sufficient for the P1, P2, and P3 scallops to fully invert into when the commissures 168 are aligned with and substantially parallel (e.g., parallel +/−10 degrees) to the edge of the flow path or outside of it, in which case the amount of scallop material left in the flow path may be very small or none. For example, the distance 275 between the commissures 168 and central axis 106 may be equal to (as shown in FIG. 18I) or greater than the distance 276 between the edge of the flow path or the edge of the annular orifice to the central axis 106, and the distance 277 between the ventricle section and the central axis 106 may be greater than both the distance 275 and 276 to provide sufficient space for the scallops to fully open (e.g., the arc length of the each scallop in closed state is less than or equal to the arc length of each scallop in open state).

The example shown in FIG. 18J is similar to that shown in FIG. 18F wherein the P1/P2 and P1/P3 leaflet connections 168 are positioned at a distance from the ventricle section 123 of the structural frame. However, in FIG. 18F a membrane layer 175 that defines a posterior aspect of each scallop 261, 262, 263 is also connected to the leaflet connections 168. In this configuration each scallop 261, 262, 263 includes a volume of space separated from one another by the membrane 175. Each volume of space is adapted to be filled with blood through an opening in the volume of space on the inferior aspect of the scallop, independently of one another. In comparison, in FIG. 18J there is no membrane separating each volume of space in the scallops and they are not separated from one another. In this configuration each volume of space is adapted to be filled with blood through an opening in the volume of space on the inferior aspect of the scallop, and/or from an opening to an adjacent scallop. The openings to adjacent scallops is defined by an empty space between leaflet connections 168 and the posterior surface of the ventricle section 123. This design may facilitate improved function of the prosthetic posterior leaflet 260 by allowing the P1, P2 and P3 scallops (261, 262, 263 respectively), to distribute filling pressure more evenly amongst themselves.

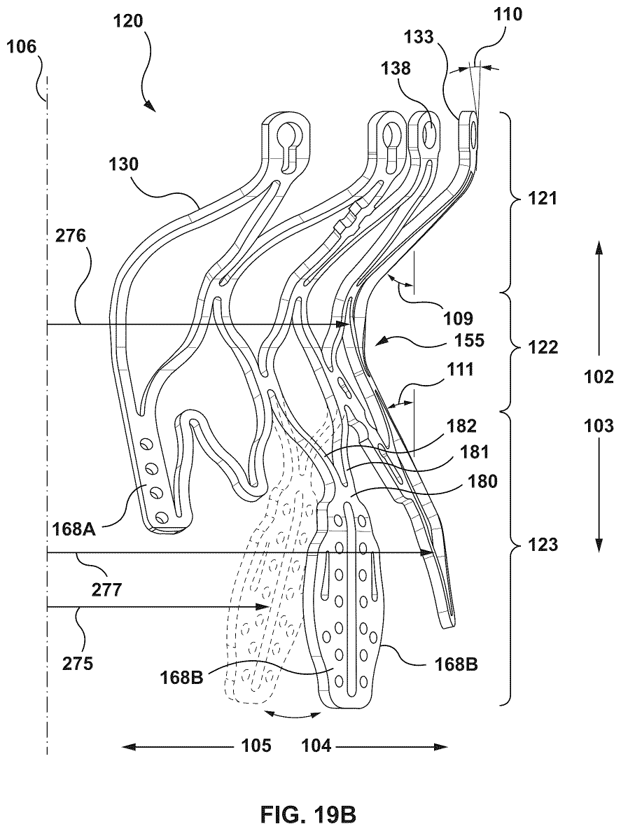

FIG. 19A, a top view, and FIG. 19B, a side view of a structural frame show an example of an arrangement as in FIG. 18H wherein the ventricle section 123 of the structural frame 120 is flared out from the annulus section 122 and wherein the P1/P2 and P2/P3 commissures 168 are positioned at a distance 275 from the central axis 106 that is shorter than the distance 277 from the central axis 106 to the curved plane 124 of the ventricle section 123, and the ventricle section is flared out, in other words distance 277 is larger than a distance 276 from the central axis 106 to the annulus section 122, and distance 275 is smaller than distance 277, for example approximately equal to distance 276, less than distance 276 (as shown), or between distances 276 and 277.

Optionally, the P1/P2 and P2/P3 commissures 168 (aka leaflet connection frames) of the structural frame may be elastically movable from a closed valve position to an open valve position, wherein the open valve position is further from the central axis 106 than the closed valve position. This may have an advantage of further reducing the amount of prosthetic valve material left in the flow path in an open state. As shown in FIG. 18G, a 2-D representation, the P1/P2 and P2/P3 commissures in a closed state 168 are closer to the central axis 106 than they are in an open state 168B; the arc length of the edge in the closed state 265A is equal to the arc length 256B between the commissures 168B in an open state allowing the scallop to lay flat and smooth in its open state and resulting in less to no remaining material in the flow path. To allow the P1/P2 and P2/P3 commissures to be elastically movable they may be connected to the rest of the structural frame in an arrangement that permits movement in the desired direction but restricts movement in other directions. For example, as shown in FIGS. 19A and 19B, the commissure 168 is connected to the rest of the frame 120 only at its superior end 180 by a connecting strut. In this case the connecting strut includes two struts 181 and 182, which are in a plane perpendicular to the bending direction 184. The Nitinol frame may be shape set with the P1/P2 and P2/P3 commissures normally in the open valve state position 168B. Blood pressure acting on the scallops to move them into the closed valve state may cause the scallops to apply a force on the commissure pulling it inward to the closed valve position 168. The elasticity of the Nitinol gently resists the pull of the scallops but allows them to close. During diastole, when the blood pressure in the scallops is released the force applied to the commissures is relieved and they elastically move back to the open valve position 168B. The amount of movement, or the difference in distance 275 from the central axis 106 to the commissures in the closed state 168 and the open state 168B may be in a range of 0 to 5 mm.

Various modifications to the implementations described in this disclosure will be readily apparent to those skilled in the art, and the generic principles defined herein can be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “example” is used exclusively herein to mean “serving as an example, instance, or illustration.” Any implementation described herein as “example” is not necessarily to be construed as preferred or advantageous over other implementations, unless otherwise stated.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. Additionally, other implementations are within the scope of the following claims. In some cases, the actions recited in the claims can be performed in a different order and still achieve desirable results.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

1. A mitral valve repair apparatus, comprising
a frame having an annulus section, an atrial section extending from a superior aspect of the frame, and a ventricle section extending from an inferior aspect of the frame, wherein the annulus section defines an arc length which is sized to be less than a major arc between native commissures of a native mitral valve annulus;
a posterior leaflet having one or more scallops connected to the frame such that the one or more scallops extend from the frame for coaptation against a native anterior leaflet when the frame is deployed;
at least one arm member connected to the frame such that the at least one arm member extends laterally in a curved configuration configured to approximate a curvature of a native mitral annulus when the frame is deployed; and
an anchoring leg connected to the frame such that the anchoring leg extends in a superior direction from a posterior side of the frame and defines a capture region between the anchoring leg and the posterior side where the capture region is sized to receive at least a portion of a native posterior leaflet in an elongated state when the frame is deployed.

2. The apparatus of claim 1, wherein the at least one arm member comprises a first arm and a second arm each extending laterally from the frame.

3. The apparatus of claim 2, wherein the first arm member comprises a first anchor point and the second arm member comprises a second anchor point where each anchor point is configured to align respectively with or in proximity to a medial and a lateral trigone of a left atrium of a subject.

4. The apparatus of claim 3, further comprising a first anchor for securing the first anchor point to the left atrium.

5. The apparatus of claim 4, wherein the first anchor comprises a corkscrew anchor and tip pillar having at least one deployable barb.

6. The apparatus of claim 5 wherein the corkscrew anchor further comprises a tip pillar.

7. The apparatus of claim 3, wherein each of the first and second anchor points comprise the medial and the lateral trigone and an area having a radius of 3 mm surrounding each of the medial and lateral trigones.

8. The apparatus of claim 3, wherein the first and second anchor points each comprise an opening for accommodating a position of the medial and the lateral trigones.

9. The apparatus of claim 8, wherein the first and second anchor points each comprise a slot for accommodating the position of the medial and the lateral trigones.

10. The apparatus of claims 1, wherein at least a portion of the mitral valve repair apparatus comprises a membrane or fabric layer.

11. The apparatus of claim 1, wherein the one or more scallops comprise a P1, P2, and P3 scallop each connected to the frame.

12. The apparatus of claim 1, wherein the posterior leaflet has a width configured to span an arc length of between 0 to 100% of the native posterior leaflet.

13. The apparatus of claim 12, wherein the width of the posterior leaflet is configured to span the arc length of between 30 to 80% of the native posterior leaflet.

14. The apparatus of claim 12, wherein the width of the posterior leaflet is configured to span the arc length of between 50 to 75% of the native posterior leaflet.

15. The apparatus of claim 1, wherein the posterior leaflet is sized to cover a native P2 scallop and a portion of a native P1 scallop and a portion of a native P3 scallop.

16. The apparatus of claim 15, wherein the posterior leaflet is sized to cover 30% to 70% of the native P1 scallop.

17. The apparatus of claim 16, wherein the posterior leaflet is sized to cover 30% to 40%, 40% to 60%, or 50% to 70% of the native P1 scallop.

18. The apparatus of claim 15, wherein the posterior leaflet is sized to cover 30% to 70% of the native P3 scallop.

19. The apparatus of claim 16, wherein the posterior leaflet is sized to cover 30% to 40%, 40% to 60%, or 50% to 70%, of the native P3 scallop.

20. The apparatus of claim 1, wherein the arc length is sized to be 30% to 90% of a circumference of the native mitral valve annulus.

21. The apparatus of claim 20, wherein the arc length is sized to be 30% to 80%, 40% to 80%, or 50% to 70% of the circumference of the native mitral valve annulus.

22. The apparatus of claim 1, wherein the posterior leaflet is configured to define pleats which separate the one or more scallops from one another.

23. The apparatus of claim 22, wherein the pleats are each comprised of a sewn border separating the one or more scallops.

24. The apparatus of claim 22, wherein the posterior leaflet is formed from a singular material shaped into the one or more scallops.

25. The apparatus of claim 22, wherein an interior of each of the one or more scallops form a continuous space with an adjacent scallop of the one or more scallops.

26. The apparatus of claim 1, wherein the at least one arm member is configured to be adjustable.

27. The apparatus of claim 26, wherein the at least one arm member is adjustable in arc length, radius of curvature, tension, compression, or flexion.

28. The apparatus of claim 26, further comprising a locking mechanism for securing a configuration of the at least one arm member.

29. The apparatus of claim 1, wherein the anchoring leg has a width sized for introduction between a chordae tendinea bundle of a lateral papillary muscle and a chordae tendinea bundle of a medial papillary muscle of a subject.

30. The apparatus of claim 29, wherein the width ranges between 2 to 8 mm.

31. The apparatus of claim 1, wherein the anchoring leg comprises a first leg attachment connected to a first portion of the frame and a second leg attachment connected to a second portion of the frame such that the anchoring leg has a middle section defining a radially outward bend.

32. The apparatus of claim 1, further comprising one or more additional leg members connected to the frame and in proximity to the anchoring leg for securing a position of the frame to the native mitral annulus.

33. The apparatus of claim 32, wherein the one or more additional leg members comprise two additional leg members positioned on each side of the anchoring leg.

34. The apparatus of claim 32, wherein each additional leg member extends from the frame at a second level which is superior to a first level of the anchoring leg.

35. The apparatus of claim 34, where a difference between the first level and the second level is between 1 mm to 10 mm.

36. The apparatus of claim 1, further comprising an arm mount delivery system for deploying the mitral valve repair apparatus.

37. The apparatus of claim 36, wherein the arm mount delivery system comprises an arm mount having configurable wing members connectable to a distal end of the at least one arm member.

38. The apparatus of claim 37, further comprising a delivery sheath connected to the arm mount via a body member from which the wing members extend.

39. The apparatus of claim 38, wherein the wing members are configurable from a delivery configuration where the wing members are retracted relative to the delivery sheath and a deployment configuration where the wing members are extended relative to the delivery sheath such that the at least one arm member is positioned into a deployment configuration.

40.-119. (canceled)
